(19) United States
(12) Patent Application Publication
(10) Pub. No.: US 2022/0175798 A1
(43) Pub. Date: Jun. 9, 2022

(54) COMPOSITIONS COMPRISING OXYGENATED CHOLESTEROL SULFATE AND AT LEAST ONE OF POLYALKYLENE GLYCOL, CARBOXYMETHYL CELLULOSE AND POLYOXYLGLYCERIDE

(71) Applicant: Durect Corporation, Cupertino, CA (US)

(72) Inventors: Andrew R. Miksztal, Palo Alto, CA (US); WeiQi Lin, Emerald Hills, CA (US); Mee Jean Kim, San Francisco, CA (US); Hongwei Wu, Cupertino, CA (US); Min L. Lee, Saratoga, CA (US); Wendy Chao, San Jose, CA (US)

(21) Appl. No.: 17/526,493

(22) Filed: Nov. 15, 2021

Related U.S. Application Data
(63) Continuation of application No. 16/320,074, filed on Jan. 23, 2019, now abandoned.
(60) Provisional application No. 62/370,200, filed on Aug. 2, 2016.
(60) Provisional application No. 62/470,834, filed on Mar. 13, 2017.

(51) Int. Cl. A61K 31/575; A61P 1/16; A61K 47/10

(52) U.S. Cl. CPC A61K 31/575; A61K 9/0053; A61K 47/10; A61P 1/16

(57) ABSTRACT

Compositions comprising oxygenated cholesterol sulfates (OCS) are provided. The OCS is, for example, 5-cholesten-3, 25-diol, 3-sulfate (25HC3S) or 5-cholesten, 3, 25-diol, disulfate (25HCDS). The compositions may be used to prevent and/or treat a variety of diseases and conditions, including organ failure (e.g. acute liver failure due to acetaminophen), high cholesterol/high lipids, and various inflammatory diseases and conditions.

This application claims the benefit of U.S. Provisional Patent Application No. 62/370,200, filed 2 Aug. 2016, and U.S. Provisional Patent Application No. 62/470,834, filed 13 Mar. 2017, which applications are incorporated herein by reference in their entirety.

### FIELD OF THE DISCLOSURE

The present disclosure generally relates to compositions comprising at least one oxygenated cholesterol sulfate (OCS). The compositions comprise at least one of polyalkylene glycol, carboxymethyl cellulose or pharmaceutically acceptable salt thereof, and polyoxylglyceride. The compositions may be used to treat and/or prophylactically treat a wide variety of diseases and conditions, such as conditions that are caused by or related to inflammation.

### INTRODUCTION

Oxygenated cholesterol sulfates (OCS) such as 5-cholesten-3, 25-diol, 3-sulfate (25HC3S) and 5-cholesten, 3, 25-diol, disulfate (25HCDS) are known to prevent or treat a wide variety of diseases and conditions. For instance, OCS's are known to be potent mediators of inflammation and are successfully used to prevent and treat diseases caused by or exacerbated by inflammation. These diseases include a wide range of maladies, for example heart disease, organ failure, etc.

There are a wide range of strategies known for formulating drugs, e.g., to maximize their therapeutic efficacy. However, it is not straightforward to predict ab initio the most appropriate strategy to apply to a new drug compound.

Compositions for improved delivery of OCS's are needed. Especially beneficial would be compositions having one or more, preferably several and most preferably all of high efficacy, low toxicity, storage stability, homogeneity, syringeability and isotonicity.

### SUMMARY

The present disclosure addresses these needs and provides compositions comprising one or more (e.g., at least one) oxygenated cholesterol sulfate (OCS). The compositions comprise at least one of polyalkylene glycol, carboxymethyl cellulose or pharmaceutically acceptable salt thereof, and polyoxylglyceride. Among other indications, the compositions may be used to prevent and treat acute liver failure. However, the use of the compositions is not limited to the treatment of acute liver failure (ALF); a variety of other diseases and conditions may also be prevented and/or treated by the compositions and methods described herein, e.g., high cholesterol/high lipids, various inflammatory diseases and conditions, organ failure of other types (e.g., kidney), etc.

Aspects of the disclosure include:

1. A composition comprising:

particles comprising one or more oxygenated cholesterol sulfates (OCS); and

a vehicle comprising at least one polyalkylene glycol, wherein the composition comprises a suspension of the particles in the vehicle.

2. The composition of aspect 1, wherein the at least one polyalkylene glycol comprises at least one polyethylene glycol.
3. The composition of aspect 1, wherein the at least one polyalkylene glycol consists of at least one polyethylene glycol.
4. The composition of any one of aspects 1 to 3, wherein the at least one polyalkylene glycol has a weight average molecular weight ranging from about 200 Daltons to about 10,000 Daltons.
5. The composition of aspect 4, wherein the at least one polyalkylene glycol has a weight average molecular weight ranging from about 300 Daltons to about 7,000 Daltons.
6. The composition of aspect 4, wherein the at least one polyalkylene glycol has a weight average molecular weight ranging from about 500 Daltons to about 5,000 Daltons.
7. The composition of any one of aspects 1 to 6, wherein the at least one polyalkylene glycol is present in an amount ranging from about 0.5 wt % to about 50 wt %, based on weight of the composition.
8. The composition of aspect 7, wherein the at least one polyalkylene glycol is present in an amount ranging from about 0.5 wt % to about 20 wt %, based on weight of the composition.
9. The composition of aspect 7, wherein the at least one polyalkylene glycol is present in an amount ranging from about 1 wt % to about 10 wt %, based on weight of the composition.
10. A composition comprising:

particles comprising one or more oxygenated cholesterol sulfates (OCS), wherein the particles have a median particle size, as measured by laser diffraction, ranging from about 0.1 μm to about 500 μm; and

a vehicle comprising at least one carboxymethyl cellulose or pharmaceutically acceptable salt thereof, wherein the composition comprises a suspension of the particles in the vehicle.

11. The composition of aspect 10, wherein the at least one carboxymethyl cellulose or pharmaceutically acceptable salt thereof has a weight average molecular weight ranging from about 50,000 Daltons to about 800,000 Daltons.
12. The composition of aspect 11, wherein the at least one carboxymethyl cellulose or pharmaceutically acceptable salt thereof has a weight average molecular weight ranging from about 70,000 Daltons to about 700,000 Daltons.
13. The composition of aspect 11, wherein the at least one carboxymethyl cellulose or pharmaceutically acceptable salt thereof has a weight average molecular weight ranging from about 80,000 Daltons to about 500,000 Daltons.
14. The composition of any one of aspects 10 to 13, wherein the at least one carboxymethyl cellulose or pharmaceutically acceptable salt thereof is present in an amount ranging from about 0.2 wt % to about 75 wt %, based on weight of the composition.
15. The composition of aspect 14, wherein the at least one carboxymethyl cellulose or pharmaceutically acceptable salt thereof is present in an amount ranging from about 0.5 wt % to about 50 wt %, based on weight of the composition.
16. The composition of aspect 14, wherein the at least one carboxymethyl cellulose or pharmaceutically acceptable salt thereof is present in an amount ranging from about 0.5 wt % to about 40 wt %, based on weight of the composition.
17. A composition comprising:

one or more oxygenated cholesterol sulfates (OCS); and

at least one polyoxylglyceride.

18. The composition of aspect 17, wherein the at least one polyoxylglyceride comprises a saturated polyglycolized glyceride.
19. The composition of aspect 18, wherein the saturated polyglycolized glyceride is a saturated polyglycolized glyceride having a melting point of from about 38° C. to about 55° C. and a hydrophilic-lipophilic balance (HLB) of from about 1 to about 16.
20. The composition of aspect 18, wherein the saturated polyglycolized glyceride is a saturated polyglycolized glyceride having a melting point of from about 38° C. to about 50° C. and an HLB of from about 1 to about 16.
21. The composition of any one of aspects 18 to 20, wherein the saturated polyglycolized glyceride is lauroyl polyoxylglycerides and/or stearoyl polyoxylglycerides.
22. The composition of any one of aspects 17 to 21, wherein the at least one polyoxylglyceride is present in the composition in an amount ranging from about 10 wt % to about 99 wt %, based on weight of the composition.
23. The composition of aspect 22, wherein the at least one polyoxylglyceride is present in the composition in an amount ranging from about 40 wt % to about 85 wt %, based on weight of the composition.
24. The composition of aspect 22, wherein the at least one polyoxylglyceride is present in the composition in an amount ranging from about 50 wt % to about 80 wt %, based on weight of the composition.
25. The composition of any one of aspects 17 to 24 and 115 to 117, wherein the composition comprises particles comprising the one or more oxygenated cholesterol sulfates.
26. The composition of aspect 25, wherein the composition comprises a suspension of the particles in a vehicle.
27. The composition of any one of aspects 1 to 9, 25 and 26, wherein the particles have a median particle size, as measured by laser diffraction, ranging from about 0.1 μm to about 500 μm.

28. The composition of any one of aspects 10 to 16 and 27, wherein the particles have a median particle size, as measured by laser diffraction, ranging from about 0.25 μm to about 50 μm.

29. The composition of aspect 28, wherein the particles have a median particle size, as measured by laser diffraction, ranging from about 0.5 μm to about 25 μm.

30. The composition of any one of aspects 1 to 29, wherein the one or more oxygenated cholesterol sulfates comprises 5-cholesten-3β, 25-diol, 3-sulfate or a pharmaceutically acceptable salt thereof.
31. The composition of any one of aspects 1 to 30, wherein the one or more oxygenated cholesterol sulfates comprises 5-cholesten, 3β, 25-diol, disulfate or a pharmaceutically acceptable salt thereof.
32. The composition of any one of aspects 1 to 29, wherein the one or more oxygenated cholesterol sulfates consists of 5-cholesten-3β, 25-diol, 3-sulfate or a pharmaceutically acceptable salt thereof.
33. The composition of any one of aspects 1 to 29, wherein the one or more oxygenated cholesterol sulfates consists of 5-cholesten, 3β, 25-diol, disulfate or a pharmaceutically acceptable salt thereof.
34. The composition of any one of aspects 1 to 33, wherein the one or more OCS is present in an amount ranging from about 0.5 wt % to about 50 wt %, based on weight of the composition.
35. The composition of aspect 34, wherein the one or more OCS is present in an amount ranging from about 0.5 wt % to about 20 wt %, based on weight of the composition.
36. The composition of aspect 34, wherein the one or more OCS is present in an amount ranging from about 1 wt % to about 10 wt %, based on weight of the composition.
37. The composition of any one of aspects 1 to 36, further comprising at least one surfactant.
38. The composition of any one of aspects 1 to 36, further comprising at least one surfactant that is a non-ionic surfactant.
39. The composition of any one of aspects 1 to 36, further comprising at least one surfactant selected from polysorbate, sorbitan ester, poloxamer, lecithin sodium dodecyl sulphate (SDS), sulphated castor oil, benzalkonicum chloride, cetrimide, polyoxyl castor oil, d-α-tocopheryl polyethylene glycol 1000 succinate (TPGS), poly-oxyethylene ester, caprylic/capric glyceride, polyglyceryl oleate, linoleic glyceride, polyoxyl stearate, peppermint oil, and oleic acid.
40. The composition of aspect 39, wherein the at least one surfactant is PEG-8 caprylic/capric glycerides and/or polyglyceryl-3 oleate.
41. The composition of any one of aspects 37 to 40, wherein the at least one surfactant is present in the composition in an amount ranging from about 0.01 wt % to about 20 wt %, based on weight of the composition.
42. The composition of any one of aspects 37 to 41, wherein the at least one surfactant is present in the composition in an amount ranging from about 0.01 wt % to about 10 wt %, based on weight of the composition.
43. The composition of any one of aspects 1 to 42, further comprising water.
44. The composition of aspect 43, wherein the water is present in an amount ranging from about 0.1 wt % to about 99 wt %, based on weight of the composition.
45. The composition of any one of aspects 1 to 44, further comprising at least one antioxidant.
46. The composition of any one of aspects 1 to 44, wherein the composition is antioxidant-free.
47. The composition of any one of aspects 1 to 46, wherein the composition is methionine-free.
48. The composition of any one of aspects 1 to 47, further comprising at least one buffer.
49. The composition of any one of aspects 1 to 48, further comprising at least one buffer selected from phosphate buffer, sodium phosphate monobasic, sodium phosphate dibasic, citrate, and borate.
50. The composition of aspect 48 or 49, wherein the at least one buffer is present in the composition at an amount ranging from about 1 mM to about 500 mM.
51. The composition of any one of aspects 1 to 50, further comprising at least one salt.
52. The composition of any one of aspects 1 to 51, further comprising at least one salt selected from sodium chloride, calcium chloride, and sodium sulfate.
53. The composition of aspect 51 or 52, wherein the at least one salt is present in an amount ranging from about 0.1 wt % to about 5 wt %, based on weight of the composition.
54. The composition of any one of aspects 1 to 53, further comprising at least one sugar.
55. The composition of any one of aspects 1 to 54, further comprising at least one sugar selected from dextrose, mannitol, and sucrose.
56. The composition of any one of aspects 1 to 55, further comprising at least one preservative.
57. The composition of any one of aspects 1 to 56, further comprising benzyl alcohol.
58. The composition of any one of aspects 1 to 57, wherein the composition further comprises glyceryl palmitostearate.
59. The composition of any one of aspects 1 to 58, wherein the composition further comprises disintegrant.
60. The composition of any one of aspects 1 to 59, wherein the composition further comprises a disintegrant that is croscarmellose sodium.
61. The composition of aspect 59 or 60, wherein the distintegrant is present in the composition in an amount ranging from about 1 wt % to about 5 wt %, based on weight of the composition.
62. The composition of any one of aspects 1 to 61, wherein the composition has an osmolality ranging from about 150 mmol/kg to about 3000 mmol/kg.
63. The composition of any one of aspects 1 to 62, wherein the composition has a pH ranging from about 3 to about 10.
64. The composition of any one of aspects 1 to 63, wherein when the composition is placed in a 1 mL syringe at 25° C. fitted with a 0.5 inch needle with a gauge of 21 and 10 lbs of force are applied, the composition is syringeable.
65. The composition of any one of aspects 1 to 64, wherein when the composition is placed in a 1 mL syringe at 25° C. fitted with a 0.5 inch needle with a gauge of 27 and 10 lbs of force are applied, the composition is syringeable.
66. The composition of any one of aspects 1 to 65, wherein the composition is contained within a bottle.
67. The composition of any one of aspects 1 to 65, wherein the composition is contained within a vial.
68. The composition of any one of aspects 1 to 67, wherein the composition is contained within a capsule.
69. The composition of aspect 68, wherein the capsule comprises gelatin.
70. The composition of aspect 68 or 69, wherein the capsule comprises hydroxypropyl methylcellulose.
71. The composition of any one of aspects 1 to 70, which comprises at least:

particles comprising one or more oxygenated cholesterol sulfates;

polyethylene glycol;

a surfactant;

a salt;

water; and

a buffer.

72. The composition of any one of aspects 1 to 71, which comprises at least:

particles comprising 25HC3S;

polyethylene glycol;

polysorbate;

NaCl;

water; and

phosphate buffer.

73. A method of treating, in a subject in need thereof, at least one of: hyperlipidemia or a disease or condition caused by hyperlipidemia; dysfunction or failure of at least one organ; a lipid metabolism disorder; metabolic disorder; atherosclerosis; injury caused by ischemia; unwanted cell death; sepsis; acute radiation syndrome; a liver disorder; a lipid accumulation disorder; a skin lesion; and an inflammatory skin disease; the method comprising administering to the subject a therapeutically effective amount of the composition of any one of aspects 1 to 72 and 105 to 133.
74. The method of aspect 73, wherein the method comprises treating dysfunction or failure of at least one organ selected from the group consisting of kidney, liver, pancreas, heart, lung and brain.
75. The method of aspect 74, wherein the method comprises treating dysfunction or failure of the liver caused by acetaminophen.
76. The method of aspect 73, wherein the method comprises treating injury caused by ischemia.
77. The method of aspect 73, wherein the method comprises treating injury caused by ischemia caused by ischemia/reperfusion injury.
78. The method of aspect 73, wherein the method comprises treating a liver disorder.
79. The method of aspect 73, wherein the method comprises treating a liver disorder that is non-alcoholic fatty liver disease (NAFLD) or nonalcoholic steatohepatitis (NASH).
80. The method of aspect 73, wherein the method comprises treating an inflammatory skin disease.
81. The method of aspect 73, wherein the method comprises treating an inflammatory skin disease that is atopic dermatitis or psoriasis.
82. The method of any one of aspects 73 to 81, wherein the administering is performed by injection.
83. The method of any one of aspects 73 to 81, wherein the administering is performed intravenously.
84. The method of any one of aspects 73 to 81, wherein the administering is performed topically.
85. The method of any one of aspects 73 to 81, wherein the administering is performed orally.
86. A method of treating, in a subject in need thereof, any disease or condition disclosed herein, the method comprising administering to the subject a therapeutically effective amount of the composition of any one of aspects 1 to 72 and 105 to 133.
87. A method of administering comprising: injecting a suspension comprising particles comprising one or more oxygenated cholesterol sulfate (OCS) suspended in a vehicle comprising a hydrophilic polymer.
88. A method of making a suspension, comprising: mixing particles comprising one or more oxygenated cholesterol sulfate (OCS) with a vehicle comprising at least one polyalkylene glycol to form a suspension.
89. A method of making a suspension, comprising: mixing particles comprising one or more oxygenated cholesterol sulfate (OCS) with a vehicle comprising at least one carboxymethyl cellulose or pharmaceutically acceptable salt thereof to form a suspension.
90. A method of making a suspension, comprising: mixing particles comprising one or more oxygenated cholesterol sulfate (OCS) with a vehicle comprising at least one polyoxylglyceride to form a suspension.
91. The method of any one of aspects 88 to 90, wherein the mixing comprises manual shaking.
92. The method of any one of aspects 88 to 91, wherein the mixing comprises sonication.
93. The method of any one of aspects 88 to 92, wherein the mixing comprises shaking in a flat bed shaker.
94. The method of any one of aspects 88 to 93, further comprising homogenizing the suspension.
95. The method of any one of aspects 88 to 94, further comprising jet milling one or more oxygenated cholesterol sulfate to form the particles.
96. The method of any one of aspects 88 to 95, further comprising sieving one or more oxygenated cholesterol sulfate to select the particles for the mixing.
97. The method of any one of aspects 88 to 96, further comprising sterilizing the particles prior to the mixing.
98. The method of any one of aspects 88 to 97, further comprising autoclaving the particles prior to the mixing.
99. The method of any one of aspects 88 to 98, further comprising gamma irradiating the particles prior to the mixing.
100. A composition as defined in any one of aspects 1 to 72 and 105 to 133 for use as a medicament.
101. A composition as defined in any one of aspects 1 to 72 and 105 to 133 for use in treatment of any disease or condition disclosed herein.
102. The composition for use of aspect 101, wherein the disease or condition is selected from hyperlipidemia or a disease or condition caused by hyperlipidemia; dysfunction or failure of at least one organ; a lipid metabolism disorder; metabolic disorder; atherosclerosis; injury caused by ischemia; unwanted cell death; sepsis; acute radiation syndrome; a liver disorder; a lipid accumulation disorder; a skin lesion; and an inflammatory skin disease.
103. Use of a composition as defined in any one of aspects 1 to 72 and 105 to 133 in the manufacture of a medicament for use in treatment of any disease or condition disclosed herein.
104. Use of aspect 103, wherein the disease or condition is selected from hyperlipidemia or a disease or condition caused by hyperlipidemia; dysfunction or failure of at least one organ; a lipid metabolism disorder; metabolic disorder; atherosclerosis; injury caused by ischemia; unwanted cell death; sepsis; acute radiation syndrome; a liver disorder; a lipid accumulation disorder; a skin lesion; and an inflammatory skin disease.
105. A composition comprising:

particles comprising 25HC3S;

lauroyl polyoxylglycerides; and

stearoyl polyoxylglycerides.

106. The composition of aspect 105, wherein the composition is in a capsule.
107. The composition of aspect 105 or 106, wherein:

the lauroyl polyoxylglycerides are present in the composition in an amount ranging from about 55 wt % to about 95 wt %, and

the stearoyl polyoxylglycerides are present in the composition in an amount ranging from about 1 wt % to about 30 wt %, based on the weight of the composition.

108. The composition of aspect 107, wherein:

the lauroyl polyoxylglycerides are present in the composition in an amount ranging from about 60 wt % to about 90 wt %, and

the stearoyl polyoxylglycerides are present in the composition in an amount ranging from about 5 wt % to about 25 wt %, based on the weight of the composition.

109. The composition of any one of aspects 105 to 108, wherein the composition comprises PEG-8 caprylic/capric glycerides.
110. The composition of any one of aspects 105 to 109, wherein the composition comprises polyglyceryl-3 oleate.
111. The composition of aspect 109 or 110, wherein the PEG-8 caprylic/capric glycerides is present in the composition in an amount ranging from about 1 wt % to about 15 wt %, based on the weight of the composition.
112. The composition of aspect 111, wherein the PEG-8 caprylic/capric glycerides is present in the composition in an amount ranging from about 5 wt % to about 10 wt %, based on the weight of the composition.
113. The composition of any one of aspects 110 to 112, wherein the polyglyceryl-3 oleate is present in the composition in an amount ranging from about 1 wt % to about 15 wt %, based on the weight of the composition.
114. The composition of any one of aspects 110 to 113, wherein the polyglyceryl-3 oleate is present in the composition in an amount ranging from about 5 wt % to about 10 wt %, based on the weight of the composition.
115. The composition of any one of aspects 17 to 20, wherein the at least one polyoxylglyceride is present in the composition in an amount ranging from about 5 wt % to about 25 wt %, based on weight of the composition.
116. The composition of any one of aspects 17 to 20, further comprising at least one polyglyceryl fatty acid ester, present in the composition in an amount ranging from about 1 wt % to about 15 wt %, based on weight of the composition.
117. The composition of any one of aspects 17 to 20, further comprising at least one polyglyceryl fatty acid ester, present in the composition in an amount ranging from about 5 wt % to about 15 wt %, based on weight of the composition.
118. A composition comprising:

particles comprising one or more oxygenated cholesterol sulfates (OCS); and

a vehicle comprising at least one polyalkylene glycol.

119. The composition of aspect 118, wherein the at least one polyalkylene glycol comprises at least one polyethylene glycol.
120. The composition of aspect 118, wherein the at least one polyalkylene glycol consists of at least one polyethylene glycol.
121. The composition of any one of aspects 118 to 120, wherein the at least one polyalkylene glycol has a weight average molecular weight ranging from about 200 Daltons to about 10,000 Daltons.
122. The composition of aspect 121, wherein the at least one polyalkylene glycol has a weight average molecular weight ranging from about 300 Daltons to about 7,000 Daltons.
123. The composition of aspect 121, wherein the at least one polyalkylene glycol has a weight average molecular weight ranging from about 500 Daltons to about 5,000 Daltons.
124. The composition of any one of aspects 118 to 123, wherein the at least one polyalkylene glycol is present in an amount ranging from about 0.5 wt % to about 50 wt %, based on weight of the composition.
125. The composition of aspect 124, wherein the at least one polyalkylene glycol is present in an amount ranging from about 0.5 wt % to about 20 wt %, based on weight of the composition.
126. The composition of aspect 124, wherein the at least one polyalkylene glycol is present in an amount ranging from about 1 wt % to about 10 wt %, based on weight of the composition.
127. A composition comprising:

particles comprising one or more oxygenated cholesterol sulfates (OCS), wherein the particles have a median particle size, as measured by laser diffraction, ranging from about 0.1 μm to about 500 μm; and

a vehicle comprising at least one carboxymethyl cellulose or pharmaceutically acceptable salt thereof.

128. The composition of aspect 127, wherein the at least one carboxymethyl cellulose or pharmaceutically acceptable salt thereof has a weight average molecular weight ranging from about 50,000 Daltons to about 800,000 Daltons.
129. The composition of aspect 128, wherein the at least one carboxymethyl cellulose or pharmaceutically acceptable salt thereof has a weight average molecular weight ranging from about 70,000 Daltons to about 700,000 Daltons.
130. The composition of aspect 128, wherein the at least one carboxymethyl cellulose or pharmaceutically acceptable salt thereof has a weight average molecular weight ranging from about 80,000 Daltons to about 500,000 Daltons.
131. The composition of any one of aspects 127 to 130, wherein the at least one carboxymethyl cellulose or pharmaceutically acceptable salt thereof is present in an amount ranging from about 0.2 wt % to about 75 wt %, based on weight of the composition.
132. The composition of aspect 131, wherein the at least one carboxymethyl cellulose or pharmaceutically acceptable salt thereof is present in an amount ranging from about 0.5 wt % to about 50 wt %, based on weight of the composition.
133. The composition of aspect 131, wherein the at least one carboxymethyl cellulose or pharmaceutically acceptable salt thereof is present in an amount ranging from about 0.5 wt % to about 40 wt %, based on weight of the composition.

### BRIEF DESCRIPTION OF THE DRAWINGS

The present invention is further described in the description of invention that follows, in reference to the noted plurality of non-limiting drawings, wherein:

FIG. 1. Osmolality vs. % NaCl Plot for the Vehicle PEG 3350 with Various % NaCl.

FIG. 2. Erythema (redness) of back skin of mice treated with 25HC3S solution, solution vehicle, 25HC3S suspension, or suspension vehicle.

FIGS. 3A and 3B. A, IL-17 and B, TNFα protein levels in psoriatic skin/lesion as measured by ELISA assays.

FIG. 4. NAFLD (non-alcoholic fatty liver disease) activity score (NAS) and fibrosis scores.

FIG. 5. Oil Red 0 Staining (black) demonstrates reduction of hepatic lipidosis by 25HC3S administration in HFD-fed hamsters.

FIG. 6. 24 hrs mean enzyme and biochemical serum levels in cohort A mice: Vehicle or 25HC3 S (25 Mg/Kg) given by oral gavage administration 1 hr after acetaminophen (APAP) (300 mg/kg) challenge.

FIG. 7. Serum Creatinine and BUN levels after 25HC3S treatment in surgically-induced kidney ischemic rats.

FIGS. 8-22. Dissolution profiles from capsule formulations tested at t=0;t=1, 3, and 7 months after storage at 25° C.; and t=0.5, 1, 3, and 7 months after storage at 40° C.

FIG. 23. NAFLD Activity Scores. Statistical test: One-way ANOVA with Dunnett's Multiple Comparisons.

FIG. 24. Percent area of fibrosis. One-way ANOVA with Dunnett's Multiple Comparisons performed. ^{a}Denotes that with Mann-Whitney test, statistical significance improves to p<0.05.

FIG. 25. Percent body weight change and absolute body temperature change on Day 9 after bile duct ligation (BDL) surgery. One-way ANOVA with Dunnett's Multiple Comparison was performed. *p<0.05; **p<0.01.

FIG. 26. Serum bilirubin levels on Day 9 after BDL surgery. One-way ANOVA with Dunnett's Multiple Comparison was performed. *p<0.05; **p<0.01; ***p<0.001.

FIG. 27. Body temperature change on Day 9 after BDL surgery. Two-way ANOVA was performed. *p<0.05.

FIG. 28. Spleen-Body weight ratio on Day 10 after BDL surgery. Student's t-test was performed. *p<0.05.

FIG. 29. Percent body weight change, body temperature and disease scores after BDL surgery. One-way ANOVA with Dunnett's Multiple Comparison was performed. *p<0.05; **p<0.01.

FIGS. 30-38. Dissolution profiles from capsule formulations tested at t=0;t=11 weeks after storage at 25° C. at 60% relative humidity; and t=2 and 11 weeks after storage at 40° C. and 75% relative humidity.

### DETAILED DESCRIPTION OF THE DISCLOSURE

Compositions comprising at least one oxygenated cholesterol sulfate (OCS) are provided. The compositions comprise at least one of polyalkylene glycol, carboxymethyl cellulose or pharmaceutically acceptable salt thereof, and polyoxylglyceride. The compositions are used to prevent and/or treat a wide variety of diseases and conditions, such as hyperlipidemia, ischemia, sepsis, heart disease, organ failure, etc.

### Definitions

The following definitions are used throughout:

As used herein, “at least one” means one, two, three, four, or more.

The compositions described herein include one or more than one OCS. Exemplary OCS's that are used in the compositions include but are not limited to: 5-cholesten-3, 25-diol, 3-sulfate (25HC3S); 5-cholesten, 3, 25-diol, disulfate (25HCDS); 5-cholestene, 3, 27-diol, 3-sulfate; 5-cholestene, 3, 27-diol, 3, 27-disulfate; 5-cholestene, 3,7-diol, 3-sulfate; 5-cholestene, 3,7-diol, 3,7-disulfate; 5-cholestene, 3, 24-diol, 3-sulfate; 5-cholestene, 3, 24-diol, 3, 24-disulfate; 5-cholestene, 3-ol, 24, 25-epoxy 3-sulfate; and salts thereof, particularly pharmaceutically acceptable salts thereof. Disclosure of 25HC3S is found in, e.g., U.S. Pat. No. 8,399,441, which is incorporated herein by reference in its entirety. Disclosure of 25HCDS is found, e.g., in US Published Application No. 20150072962, which is incorporated by reference in its entirety. In certain aspects, the OCS is selected from 5-cholesten-3, 25-diol, 3-sulfate (25HC3S) and 5-cholesten, 3, 25-diol, disulfate (25HCDS) (either alone or in combination). In further aspects, the OCS is 5-cholesten-3, 25-diol, 3-sulfate (25HC3S).

The OCS's are typically synthetic versions of OCS that occur naturally in the body. The OCS may be administered in forms not naturally found in the body, and in concentrations that are significantly higher than those which occur naturally. For 25HC3S, natural levels typically range from e.g. about 2 ng/ml or less up to about 5 ng/ml in the blood or plasma. The concentration of OCS (e.g. 25HC3S) in the blood or plasma of a patient that is treated with an OCS (e.g. 25HC3S) is generally greater than about 5 ng/ml, and generally ranges from about 50 ng/ml to about 5000 ng/ml, such as about 80 ng/ml to about 3000 ng/ml, e.g. from about 100 to about 2000 ng/ml, or from about 200 to about 1000 ng/ml.

In one aspect, the OCS is 5-cholesten-3, 25-diol, 3-sulfate (25HC3S) of formula

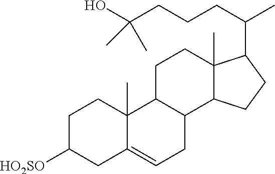

and/or a pharmaceutically acceptable salt thereof.

In one aspect, the OCS is 5-cholesten-3β, 25-diol, 3-sulfate of formula

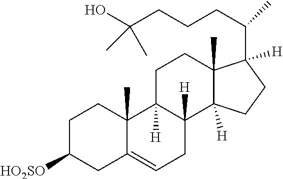

and/or a pharmaceutically acceptable salt thereof.

In one aspect, the OCS is 5-cholesten, 3, 25-diol, disulfate (25HCDS) of the formula

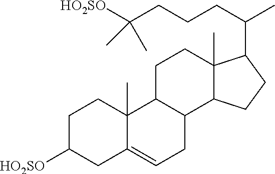

and/or a pharmaceutically acceptable salt thereof.

In some aspects, the OCS is 5-cholesten, 3β, 25-diol, disulfate of the formula

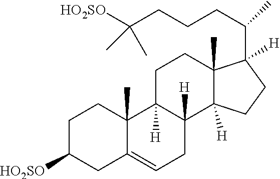

and/or a pharmaceutically acceptable salt thereof.

In some aspects, the one or more oxygenated cholesterol sulfates comprises 5-cholesten-3, 25-diol, 3-sulfate (25HC3S) or a pharmaceutically acceptable salt thereof. In some aspects, the one or more oxygenated cholesterol sulfates comprises 5-cholesten, 3, 25-diol, disulfate (25HCDS) or a pharmaceutically acceptable salt thereof. In some aspects, the one or more oxygenated cholesterol sulfates consists of 5-cholesten-3, 25-diol, 3-sulfate (25HC3S) or a pharmaceutically acceptable salt thereof. In some aspects, the one or more oxygenated cholesterol sulfates consists of 5-cholesten, 3, 25-diol, disulfate (25HCDS) or a pharmaceutically acceptable salt thereof.

### Prevent and Treat

As used herein, “prophylactically treat” (“prophylactic treatment”, “prophylactically treating” etc.) and “prevent” (“prevention”, “preventing” etc.) refer to warding off or averting the occurrence of at least one symptom of a disease or unwanted condition (such as ALF or another disease or condition described herein), by prophylactic administration of a composition comprising at least one OCS and at least one of polyalkylene glycol, carboxymethyl cellulose or pharmaceutically acceptable salt thereof, and polyoxyglyceride, to a subject in need thereof. Generally, “prophylactic” or “prophylaxis” relates to a reduction in the likelihood of the patient developing a disorder. Typically, the subject is considered by one of skill in the art to be at risk of or susceptible to developing at least one symptom of the disease or unwanted condition, or is considered to be likely to develop at least one symptom of the disease/condition in the absence of medical intervention. Generally, however, for “prevention” or “prophylactic treatment”, administration occurs before the subject has, or is known or confirmed to have, symptoms of the disease (condition, disorder, syndrome, etc.; unless otherwise indicated, these terms are used interchangeably herein). In other words, symptoms may not yet be overt or observable. The subject may be considered at risk due to a variety of factors, including but not limited to: genetic predisposition; an impending medical or surgical procedure (e.g. surgery, use of a contrast dye in imaging, chemotherapy, etc.); recent certain or suspected or unavoidable future exposure to a toxic agent (e.g. a toxic chemical or medication, radiation, etc.); or exposure to or experience of another stressor or combination of stressors that is/are linked to or associated with the development of the disease/condition which is being prevented. For example, in some aspects, what is prevented is organ dysfunction/failure (e.g. ALF), and the subject may already display symptoms of a potential precursor of organ dysfunction/failure, for example, ischemia, sepsis, a harmful or inappropriate level of inflammation, deleterious cell death, necrosis, etc. In such aspects, treatment of the subject may prevent the noxious or harmful effects or outcomes (results) of the precursor condition, for example, the treatment may prevent death. “Prevention” or “prophylactic treatment” of a disease or condition may involve completely preventing the occurrence of detectable symptoms, or, alternatively, may involve lessening or attenuating the degree, severity or duration of at least one symptom of the disease that would occur in the absence of the medical interventions provided herein. Alternatively, the subject may be experiencing early stage symptoms and what is prevented is the progression to full-blown disease.

“Treat” (treatment, treating, etc.) as used herein refers to administering at least one composition comprising OCS and at least one of polyalkylene glycol, carboxymethyl cellulose or pharmaceutically acceptable salt thereof, and polyoxylglyceride, to a subject that already exhibits at least one symptom of a disease. In other words, at least one parameter that is known to be associated with the disease has been measured, detected or observed in the subject. For example, some organ dysfunction/failure and/or precursors thereof that are treated as described herein are caused by somewhat predictable factors (e.g. APAP overdose), or by unexpected causes such as trauma due to accidents (recreational and non-recreational), war, undiagnosed allergies or other risk factors, etc. “Treatment” of a disease involves the lessening or attenuation, or in some instances, the complete eradication, of at least one symptom of the disease that was present prior to or at the time of administration of the composition. Thus, for example, treatment of ALF includes treating damage associated with ALF.

APAP overdose: Generally, a serum plasma concentration of APAP of 140-150 microgram/mL (or milligrams/L) at 4 hours post ingestion, on the Rumack-Matthew nomogram, indicates the need for APAP overdose treatment. The Rumack-Matthew nomogram is a logarithmic graph starting not directly from ingestion, but from 4 hours post ingestion after absorption is considered likely to be complete. However, the nomogram is not used alone if the patient has altered mental status (e.g. is suicidal) or if the history is not reliable. Rather, a second level is drawn and plotted to see if the slope of the line remains at or above the nomogram. A formal half-life may also be determined, e.g. by measuring APAP blood levels at time (t=0) (upon admission of the patient) and at time (t=4 hrs). If the half-life is more than 4 hours, then treatment is likely necessary to prevent hepatotoxicity and liver failure. However, treatment may be undertaken at lower blood plasma levels if deemed warranted, e.g. in a child or the elderly, as some persons are especially sensitive to APAP. Generally, if more than 4000 mg of APAP is ingested in a 24 hour period, an overdose might be suspected. Ingestion of 7000 mg or more can lead to a severe overdose if not treated. Symptoms of an overdose include: abdominal pain, appetite loss, coma, convulsions, diarrhea, irritability, jaundice, nausea, sweating, upset stomach, and vomiting, each of which may be prevented or treated by administration of the compositions described herein.

As used herein, “syringeable” refers to the ability to both fill and expel a composition from a needle and syringe.

As used herein, “suspension” means that drug particles remain suspended in the suspension vehicle such that dose uniformity is obtainable, as determined from aliquots drawn volumetrically, during a stationary room temperature storage period of 8 hours after the suspension is prepared. The suspension may exhibit substantially uniform drug particle dispersion and substantially no phase separation during a stationary room temperature storage period of 8 hours after preparation.

The term “dose uniformity” herein means that, with respect to aliquots drawn volumetrically from the same suspension, either drawn simultaneously or at different time points and drawn from the same or different locations within the suspension, all aliquots contain substantially similar amounts (i.e. ±about 15%) of suspended drug and substantially similar amounts of free drug. An amount of drug in a given volume of suspension can be measured by any suitable method, for example by high performance liquid chromatography.

### Compositions

The compositions described herein generally comprise at least one OCS and at least one of polyalkylene glycol, carboxymethyl cellulose or pharmaceutically acceptable salt thereof, and polyoxylglyceride. In some aspects, the one or more OCS is present in the composition in an amount ranging from about 0.01 to about 75% (w/w), e.g., about 0.1 to about 50% (w/w), about 1 to about 25% (w/w), about 2 to about 20% (w/w), or about 3 to about 10% (w/w).

The one or more oxygenated cholesterol sulfate is typically present in an amount ranging from about 0.5 wt % to about 50 wt %, such as about 0.5 wt % to about 30 wt %, about 0.5 wt % to 20 wt %, about 0.5 wt % to about 10 wt %, about 1 wt % to about 15 wt %, about 1 wt % to about 10 wt %, about 1 wt % to about 5 wt %, about 1 wt % to about 4 wt %, or about 1 wt % to 3 wt %, based on weight of the composition.

If a single (only one) OCS (e.g. 25HC3S or 25HCDS) is present in a liquid, lotion, or cream composition (including liquid solutions, suspensions, such as liquid suspensions, lotions, creams, etc.), the concentration of the OCS generally ranges from about 0.01 to about 200 mg/ml, or from about 0.1 to 100 mg/ml, and is generally from about 1 to about 50 mg/ml, e.g. is about 1, 5, 10, 15, 20, 25, 30, 35, 40, 45, or 50 mg/ml. If multiple OCS's are present (e.g. 2 or more, such as 2, 3, 4, 5, or more) in a solution composition, the concentration of each typically ranges from about 0.01 to about 200 mg/ml, or from about 0.1 to 100 mg/ml, and generally from about 1 to about 50 mg/ml, e.g. is about 1, 5, 10, 15, 20, 25, 30, 35, 40, 45, or 50 mg/ml.

If a single (only one) OCS (e.g. 25HC3S or 25HCDS) is present in a solid or semi-solid composition (e.g., a gel or other solidified preparation), the concentration of the OCS generally ranges from about 0.01 to about 75% (w/w) or from about 0.1 to about 50% (w/w), and is generally from about 1 to about 25% (w/w), e.g. is about 1, 5, 10, 15, 20, 25, 30, 35, 40, 45, or 50% (w/w). If multiple OCS's are present (e.g. 2 or more, such as 2, 3, 4, 5, or more) are present in a solid or semi-solid composition, the concentration of each typically ranges from about 0.01 to about 75% (w/w) or from about 0.1 to about 50% (w/w), and is generally from about 1 to about 25% (w/w), e.g. is about 1, 5, 10, 15, 20, 25, 30, 35, 40, 45, or 50% (w/w).

If a single (only one) OCS (e.g. 25HC3S or 25HCDS) is present in a lyophilized solid composition, the concentration of the OCS generally ranges from about 0.01 to about 100% (w/w), about 0.1 to about 75% (w/w), and may range from about 1 to about 15% (w/w), e.g. is about 1, 2, 3, 4, 5, 6, 7, 8, 9, 10, 11, 12, 13, 14, or 15% (w/w. If multiple OCS's are present (e.g. 2 or more, such as 2, 3, 4, 5, or more) in a lyophilized solid composition, the concentration of each typically ranges from about 0.01 to about 15% (w/w), and generally from about 1 to about 11% (w/w), e.g. is about 1, 2, 3, 4, 5, 6, 7, 8, 9, 10 or 11%.

### Particle Size

The particles comprising the one or more OCS, which are used, e.g., to make the disclosed particle-containing compositions, typically have a median particle size, as measured by laser diffraction, ranging from 0.1 micrometer to 500 micrometers, such as 0.2 micrometer to 50 micrometers, 0.25 micrometer to 50 micrometers, 0.1 micrometer to 25 micrometers, 0.1 micrometer to 10 micrometer, 0.2 micrometer to 10 micrometers, 0.5 micrometers to 10 micrometers, 0.5 micrometers to 25 micrometers, 0.5 micrometer to 7 micrometers, or 1 micrometer to 5 micrometers, 2 micrometers to 7 micrometers, or 3 micrometers to 5 micrometers. When the composition is for injection, the particles tend to have a median particle size, as measured by laser diffraction, ranging from about 0.5 μm to about 25 μm, such as about 1 μm to about 20 μm, about 2 μm to about 7 μm, or about 3 μm to about 5 μm.

The particles comprising the one or more OCS, which are used, e.g., to make the disclosed particle-containing compositions, typically have a D₉₀ particle size, as measured by laser diffraction, ranging from 0.1 micrometer to 1000 micrometers, such as 0.2 micrometer to 500 micrometers, 0.25 micrometer to 250 micrometers, 0.1 micrometer to 150 micrometers, 0.1 micrometer to 100 micrometer, 0.2 micrometer to 75 micrometers, 0.5 micrometers to 60 micrometers, 0.5 micrometers to 50 micrometers, 0.5 micrometer to 40 micrometers, or 1 micrometer to 30 micrometers, 2 micrometers to 20 micrometers, or 3 micrometers to 10 micrometers. When the composition is for injection, the particles tend to have a D₉₀ particle size, as measured by laser diffraction, ranging from about 0.5 μm to about 50 μm, such as about 1 μm to about 30 μm, about 2 μm to about 20 μm, or about 3 μm to about 10 μm.

When particles are relatively large, e.g., median particle size, as measured by laser diffraction, e.g., a median particle size, as measured by laser diffraction, above 20 micrometers, the particles have a tendency to fall out of suspension in lower viscosity formulations. When particles are relatively small, the particles are relatively difficult to handle. The particle size may also affect bioavailability.

In the context of the present disclosure, unless specified to the contrary, the median particle size, as measured by laser diffraction, refers to the size of the particles before addition with the vehicle. Thus, the recited particle-containing compositions are “made from” or “obtainable by combining” the particles comprising the pharmaceutical active agent and the one or more further specified components.

In the final particle-containing composition, the particles comprising the one or more OCS may have a median particle size, as measured by laser diffraction, ranging from 0.1 micrometer to 500 micrometers, such as 0.2 micrometer to 50 micrometers, 0.25 micrometer to 50 micrometers, 0.1 micrometer to 25 micrometers, 0.1 micrometer to 10 micrometer, 0.2 micrometer to 10 micrometers, 0.5 micrometers to 10 micrometers, 0.5 micrometers to 25 micrometers, 0.5 micrometer to 7 micrometers, or 1 micrometer to 5 micrometers, 2 micrometers to 7 micrometers, or 3 micrometers to 5 micrometers. When the composition is for injection, the particles tend to have a median particle size, as measured by laser diffraction, ranging from about 0.5 μm to about 25 such as about 1 μm to about 20 about 2 μm to about 7 or about 3 μm to about 5

### Polyalkylene Glycol

The present compositions may include a polyalkylene glycol, e.g., at least one polyalkylene glycol as described herein. Polyalkylene glycol is a polymer containing a repeating unit [—O—alkylene-]. The alkylene may be substituted by lower alkyl or hydroxyl. Preferred examples of the polyalkylene glycol are polymers consisting of C2-3 alkylene chains, and more preferred examples thereof are polyethylene glycol and polypropylene glycol. The polyalkylene glycol may be any of straight-chain, stellate and branched. In some aspects, the polyalkylene glycol is a polyether glycol, such as poly(ethylene glycol) PEG, poly(propylene glycol) PPG, and/or poly(tetramethylene glycol) PTMEG. At least one polyalkylene glycol as described herein may be included in the present compositions in combination with at least one of carboxymethyl cellulose (or pharmaceutically acceptable salt thereof) and polyoxylglyceride as described herein.

In some aspects, the at least one polyalkylene glycol comprises at least one polyethylene glycol. The term “PEG” or “polyethylene glycol” means a polymer comprising repeating units of compounds containing —(O—CH2—CH2)—. In some aspects, the at least one polyalkylene glycol consists of at least one polyethylene glycol.

The term “Multi-Arm PEG” refers to PEGs that are formed around a core molecule permitting multiple PEG molecules to be covalently bonded to the core. A multi-arm PEG includes a 4-arm PEG, a 6-arm PEG or any PEG having multiple PEGs attached to a core molecule.

The term “Multi-Branch PEG” refers to a single PEG polymer having in-chain epoxide moieties attached thereto. Multi-branched PEGs may be characterized by having a particular ratio of epoxide:ethylene oxide moieties. A fully derivatized multi-branch PEG will have an epoxide:ethylene oxide ratio of 2. However, it should be understood that multi-branch PEGs may have epoxide:ethylene oxide ratios of less than 2, and that the ratio, on average, need not be integral in a plurality of PEG molecules.

The at least one polyalkylene glycol typically has a weight average molecular weight ranging from about 200 Daltons to about 10,000 Daltons, such as about 300 Daltons to about 7000 Daltons, or about 500 Daltons to about 5000 Daltons.

The at least one polyalkyelene glycol is typically present in an amount ranging from about 0.2 wt % to about 75 wt %, such as from about 0.5 wt % to about 50 wt %, about 0.5 wt % to about 40 wt %, about 0.5 wt % to about 20 wt %, or about 1 wt % to about 10 wt %, based on weight of the composition.

### Carboxymethyl Cellulose

The present compositions may include carboxymethyl cellulose or pharmaceutically acceptable salt thereof, e.g., at least one carboxymethyl cellulose or pharmaceutically acceptable salt thereof as described herein. Pharmaceutically acceptable salts of carboxymethylcellulose include sodium carboxymethylcellulose or other alkali metal or alkaline earth metal salts of carboxymethylcellulose. For instance, the term “carboxymethyl cellulose or pharmaceutically acceptable salt thereof” as used herein encompasses cellulose substituted with groups of the formula —CH2CO2A, wherein A is hydrogen or a monovalent cation, such as K+ or preferably Na+. At least one carboxymethyl cellulose (or pharmaceutically acceptable salt thereof) as described herein may be included in the present compositions in combination with at least one of polyalkylene glycol and polyoxylglyceride as described herein.

In some aspects, the at least one carboxymethyl cellulose or pharmaceutically acceptable salt thereof has a weight average molecular weight ranging from about 50,000 Daltons to about 800,000 Daltons, such as about 70,000 Daltons to about 700,000 Daltons or about 80,000 Daltons to about 500,000 Daltons. In some aspects, the at least one carboxymethyl cellulose or pharmaceutically acceptable salt thereof is present in an amount ranging from about 0.2 wt % to about 75 wt %, such as from about 0.5 wt % to about 50 wt %, about 0.5 wt % to about 40 wt %, about 0.5 wt % to about 20 wt %, or about 1 wt % to about 10 wt %, based on weight of the composition.

### Polyoxylglyceride

The present compositions may include a polyoxyglyceride, e.g., at least one polyoxyglyceride as described herein. For example, in some embodiments, the composition comprises at least one polyoxyglyceride, e.g., caprylocaproyl polyoxylglycerides, lauroyl polyoxylglycerides, linoleoyl polyoxylglycerides, oleoyl poloxylglycerides, stearoyl polyoxylglycerides, and Gelucire®s (saturated polyglycolized glyceride (e.g., Gattefosse brand)) and Labrasol® (Gattefosse brand). At least one polyoxyglyceride as described herein may be included in the present compositions in combination with at least one of polyalkylene glycol and carboxymethyl cellulose (or pharmaceutically acceptable salt thereof) as described herein.

In some aspects, the at least one polyoxylglyceride is present in the composition in an amount ranging from about 10 wt % to about 99 wt %, such as about 40 wt % to about 85 wt %, or about 50 wt % to about 80 wt %, based on weight of the composition.

In some embodiments, the composition includes one or more Gelucire®s (saturated polyglycolized glycerides) and/or Labrasol® (PEG-8 caprylic/capric glycerides) (e.g., glycerol esters of saturated C8-C10 fatty acids). Suitable Gelucire®s include, e.g., Gelucire® 44/14 (lauroyl polyoxylglycerides), Gelucire® 43/01 (hard fat EP/NF/JPE), Gelucire® 39/01 (glycerol esters of fatty acids, e.g., glycerol esters of saturated C12-C18 fatty acids), Gelucire® 48/16 (Polyoxyl stearate (Type I) NF), and Gelucire® 50/13 (stearoyl polyoxylglycerides). Accordingly, in some embodiments, a Gelucire®, e.g., Gelucire® 44/14, Gelucire® 43/01, Gelucire® 39/01, Gelucire® 48/16, Gelucire® 50/13, Labrasol® or a combination thereof, is present in the compositions of the present disclosure at from about 10 to about 99 percent by weight relative to the weight of the composition (wt %), e.g., from about 40 to about 85 wt %, from about 50 to about 80 wt %, from about 55 to about 75 wt %, or from about 60 to about 70 wt %. In some embodiments, a Gelucire®, e.g., Gelucire® 44/14, Gelucire® 43/01, Gelucire® 39/01, Gelucire® 48/16, Gelucire® 50/13, or Labrasol®, or a combination thereof, is present in the composition of the present disclosure at about 5 wt %, about 10 wt %, about 15 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 99 wt %, relative to the weight of the composition. In some embodiments, a Gelucire®, e.g., Gelucire® 44/14, Gelucire® 43/01, Gelucire® 39/01, Gelucire® 48/16, Gelucire® 50/13, or Labrasol®, or a combination thereof, is present in the compositions of the present disclosure at from about 5 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, or about 90 wt % to about 99 wt %, relative to the weight of the composition. In some embodiments, the composition includes Gelucire® 44/14 at from about 60 wt % to about 90 wt % (e.g., about 65 wt % to about 85 wt %) and Gelucire® 50/13 at from about 1 wt % to about 20 wt % (e.g., about 5 wt % to about 15 wt %), relative to the weight of the composition. In some embodiments, the composition includes Gelucire® 44/14, Gelucire® 50/13, and/or Labrasol at a weight percent equal or approximately equal to that shown in Table 28.

Each Gelucire is designated by two numbers separated by a slash, the first number (two-digit number) indicating its melting point and the second, the HLB (hydrophilic-lipophilic balance).

In some embodiments, the composition comprises a saturated polyglycolized glyceride having a melting point of from about 38° C. to about 55° C. or 39° C. to about 50° C. (e.g., about 40° C., about 41° C., about 42° C., about 43° C., about 44° C., about 45° C., about 46° C., about 47° C., about 48° C., or about 49° C.) and an HLB of from about 1 to about 16 (e.g., about 2, about 3, about 4, about 5, about 6, about 7, about 8, about 9, about 10, about 11, about 12, about 13, about 14, or about 15). Accordingly, in some embodiments, a saturated polyglycolized glyceride having a melting point of from about 38° C. to about 55° C. or 38° C. to about 50° C. (e.g., about 39° C., about 40° C., about 41° C., about 42° C., about 43° C., about 44° C., about 45° C., about 46° C., about 47° C., about 48° C., or about 49° C.) and an HLB of from about 1 to about 16 (e.g., about 2, about 3, about 4, about 5, about 6, about 7, about 8, about 9, about 10, about 11, about 12, about 13, about 14, or about 15) is present in the compositions of the present disclosure at from about 0.01 to about 99 percent by weight relative to the weight of the drug composition (wt %), e.g., from about 10 to about 99 wt %, from about 40 to about 85 wt %, from about 50 to about 80 wt %, from about 55 to about 75 wt %, or from about 60 to about 70 wt %. In some embodiments, a saturated polyglycolized glyceride having a melting point of from about 38° C. to about 55° C. or 38° C. to about 50° C. (e.g., about 39° C., about 40° C., about 41° C., about 42° C., about 43° C., about 44° C., about 45° C., about 46° C., about 47° C., about 48° C., or about 49° C.) and an HLB of from about 1 to about 16 (e.g., about 2, about 3, about 4, about 5, about 6, about 7, about 8, about 9, about 10, about 11, about 12, about 13, about 14, or about 15) is present in the composition of the present disclosure at about 5 wt %, about 10 wt %, about 15 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 99 wt %, relative to the weight of the composition. In some embodiments, a saturated polyglycolized glyceride having a melting point of from about 38° C. to about 55° C. or 38° C. to about 50° C. (e.g., about 39° C., about 40° C., about 41° C., about 42° C., about 43° C., about 44° C., about 45° C., about 46° C., about 47° C., about 48° C., or about 49° C.) and an HLB of from about 1 to about 16 (e.g., about 2, about 3, about 4, about 5, about 6, about 7, about 8, about 9, about 10, about 11, about 12, about 13, about 14, or about 15) is present in the composition of the present disclosure at from about 5 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, or about 90 wt % to about 99 wt %, relative to the weight of the composition.

In some embodiments, the composition comprises at least one polyglyceryl fatty acid ester, e.g., Plurol® Oleique CC 497 (Polyglyceryl-3 oleate), wherein the polyglyceryl fatty acid ester is present in the composition of the present disclosure at from about 1 wt % to about 15 wt %, about 5 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, or about 90 wt % to about 99 wt %, relative to the weight of the composition. In some embodiments, the composition comprises at least one polyglyceryl fatty acid ester, e Plurol Oleique CC 497 (Polyglyceryl-3 oleate), wherein the polyglyceryl fatty acid ester is present in the composition of the present disclosure at about 1 wt %, about 5 wt %, about 10 wt %, about 15 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 99 wt %, relative to the weight of the composition. In some embodiments, the composition comprises at least one polyglyceryl fatty acid ester, e.g., Plurol Oleique CC 497 (Polyglyceryl-3 oleate) at a weight percent equal or approximately equal to that shown in Table 28.

Without being bound by theory, it is believed that polyoxylglycerides tend to increase the bioavailability of the OCS. Although the OCS may be water insoluble, formulations comprising a polyoxylglyceride may help deliver the OCS in a solubilized state. The polyoxylglyceride may increase absorption by triggering fed state conditions, increasing permeability across enterocytes, and/or promoting lymphatic transport.

The compositions are generally administered in a pharmaceutically acceptable formulation which includes suitable excipients, elixirs, binders, and the like (generally referred to as “pharmaceutically and physiologically acceptable carriers”), which are pharmaceutically acceptable and compatible with the active ingredients. Drug carriers may also be used to improve the pharmacokinetic properties, specifically the bioavailability, of many drugs with poor water solubility and/or membrane permeability.

The OCS and at least one of polyalkylene glycol, carboxymethyl cellulose or pharmaceutically acceptable salt thereof and polyoxylglyceride, may be present in the formulation as pharmaceutically acceptable salts (e.g. alkali metal salts such as sodium, potassium, calcium or lithium salts, ammonium, etc.) or as other complexes. It should be understood that the pharmaceutically acceptable formulations include solid, semi-solid, and liquid materials conventionally utilized to prepare solid, semi-solid and liquid dosage forms such as tablets, capsules, creams, lotions, ointments, gels, foams, pastes, aerosolized dosage forms, and various injectable forms (e.g. forms for intravenous administration), etc. Suitable pharmaceutical carriers include but are not limited to inert solid diluents or fillers, sterile aqueous solutions and various organic solvents for parenteral use, such as polyethylene glycol (PEG, such as PEG 300 and PEG 400), ethanol, benzyl alcohol, benzyl benzoate, propylene glycol, N,N-dimethylacetamide, N-methyl-2-pyrrolidone, vegetable oils (sesame, soybean, corn, castor, cottonseed, and peanut) and glycerin. Examples of solid carriers (diluents, excipients) include lactose, starch, conventional disintegrating agents, coatings, lactose, terra alba, sucrose, talc, gelatin, agar, pectin, acacia, magnesium stearate, stearic acid and lower alkyl ethers of cellulose. Examples of liquid carriers include but are not limited to various aqueous or oil based vehicles, saline, dextrose, glycerol, ethanol, isopropanol, phosphate buffer, syrup, peanut oil, olive oil, phospholipids, fatty acids, fatty acid amines, polyoxyethylene, isopropyl myristate, ethyl cocoate, octyl cocoate, polyoxyethylenated hydrogenated castor oil, paraffin, liquid paraffin, propylene glycol, celluloses, parabens, stearyl alcohol, polyethylene glycol, isopropyl myristate, phenoxyethanol, and the like, or combinations thereof. Water may be used as the carrier for the preparation of compositions which may also include conventional buffers and agents to render the composition isotonic. Oral dosage forms may include various thickeners, flavorings, diluents, emulsifiers, dispersing aids, binders, coatings and the like. The composition of the present disclosure may contain any such additional ingredients so as to provide the composition in a form suitable for the intended route of administration. In addition, the composition may contain minor amounts of auxiliary substances such as wetting or emulsifying agents, pH buffering agents, and the like. Similarly, the carrier or diluent may include any sustained release material known in the art, such as glycerol monostearate or glycerol distearate, alone or mixed with wax. Other potential additives and other materials (preferably those which are generally regarded as safe [GRAS]) include: colorants; flavorings; surfactants (e.g., non-ionic surfactants including polysorbate (such as TWEEN®20, 40, 60, and 80 polyoxyethylene sorbitan monolaurate), sorbitan esters (such as Span 20, 40, 60, and 85), and poloxamers (such as Pluronic L44, Pluronic F68, Pluronic F87, Pluronic F108 and Pluronic F127); zwitterionic surfactant such as lecithin; anionic surfactants such as sodium dodecyl sulphate (SDS) and sulphated castor oil; and cationic surfactants such as benzalkonicum chloride and cetrimide. Surfactants include polyoxyl 35 castor oil (Cremophor EL), polyoxyl 40 hydrogenated castor oil (Cremophor RH 40), polyoxyl 60 hydrogenated castor oil (Cremophor RH 60), d-α-tocopheryl polyethylene glycol 1000 succinate (TPGS), poly-oxyethylene esters of 12-hydroxystearic acid (Solutol HS-15), PEG 300 caprylic/capric glycerides (Softigen 767), PEG 400 caprylic/capric triglycerides (Labrafil M-1944CS), PEG-8 caprylic/capric glycerides (Labrasol®), polyglyceryl oleate (e.g., polyglyceryl-3 oleate (Plurol® CC497)), PEG 300 linoleic glycerides (Labrafil M-2125CS), polyoxyl 8 stearate (PEG 400 monostearate), polyoxyl 40 stearate (PEG 1750 monostearate), peppermint oil, oleic acid, etc.); and solvents, stabilizers, binders or encapsulants (lactose, liposomes, etc.). Preservatives such as benzyl alcohol, phenol, chlorobutanol, 2-ethoxyethanol, methyl paraben, ethyl paraben, propyl paraben, benzoic acid, sorbic acid, potassium sorbate, chlorhexidine, 3-cresol, thimerasol, phenylmercurate salts, sodium benzoate, cetrimonium bromide, benzethonium chloride, alkyltrimethylammonium bromide, cetyl alcohol, steryl alcohol, chloroactamide, trichlorocarban, bronopol, 4-chlorocresol, 4-chloroxylenol, hexachloropherene, dichlorophene, or benzalkium chloride may also be used. Depending on the formulation, it is expected that the active components (e.g. at least one OCS) will each be present at about 1 to about 99% (w/w) of the composition and the vehicular “carrier” will constitute about 1 to about 99% (w/w) of the composition. The pharmaceutical compositions of the present disclosure may include any suitable pharmaceutically acceptable additives or adjuncts to the extent that they do not hinder or interfere with the therapeutic effect(s) of the composition. Still other suitable formulations for use in the present disclosure can be found, for example in Remington's Pharmaceutical Sciences 22nd edition, Allen, Loyd V., Jr editor (September 2012); and Akers, Michael J. Sterile Drug Products: Formulation, Packaging, Manufacturing and Quality; publisher Informa Healthcare (2010).

In addition, formulations used for the treatment of ALF optionally also include additional suitable co-formulated (or optionally, co-administered) agents that are used to e.g. combat acetaminophen toxicity, including but not limited to: metabolites of the methionine and/or glutathione biosynthetic pathways such as S-adenosylhomocysteine (SAH), S-methylmethionine (SMM), cystine, betaine, etc. or various forms and/or salts thereof e.g. acetylcysteine (e.g. intravenous N-acetylcysteine), as well as various neutraceuticals, activated charcoal, etc. For example, a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, may optionally include additional suitable co-formulated (or optionally, co-administered) agents that are used to e.g. combat acetaminophen toxicity.

In some aspects, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises at least one surfactant. In some cases, the composition further comprises at least one non-ionic surfactant. Examples of surfactants include, but are not limited to, at least one surfactant selected from polysorbate, Triton X100, and SDS. In some cases, the at least one surfactant is present in the composition in an amount ranging from about 0.01 wt % to about 20 wt %, such as about 0.01 wt % to about 10 wt %, about 0.01 wt % to about 5 wt %, about 0.03 wt % to about 2 wt %, about 0.1 wt % to about 0.3 wt %, or about 0.05 wt % to about 10 wt %, based on weight of the composition. In some cases, the at least one surfactant is present in the composition in an amount ranging from about 5 wt % to about 10 wt %, such as about 6 wt % to about 10 wt %, about 7 wt % to about 10 wt %, about 8 wt % to about 10 wt %, or about 9 wt % to about 10 wt %, based on the weight of the composition.

The composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, may further comprise water. The water is typically present in an amount ranging from about 0.1 wt % to about 99 wt %, such as about 0.05 wt % to about 98 wt %, about 70 wt % to about 98 wt %, about 80 wt % to about 97 wt %, about 90 wt % to about 96 wt %, or about 1 wt % to about 10 wt %, based on weight of the composition.

In some cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises at least one antioxidant. Examples of antioxidants include, but are not limited to, methionine, BHT, BHA, ascorbic acid, ascorbyl palmitate, acetylcysteine, vitamin A, sodium metabisulfite, sodium thiosulfate, propyl gallate, and vitamin E. In other case, the composition is antioxidant-free. For instance, the composition may be methionine-free.

In some aspects, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, contains a pharmaceutically acceptable buffer, or buffers, such as phosphate, acetate, ammonia, borate, citrate, carbonate, glycine, lactate, lysine, maleic, succinate, tartrate or tromethamine. In some aspects, the buffer concentrations in the composition range from about 0.1 to about 200 mM, in some aspects they range from about 1 to about 50 mM, and in some aspects, they range from about 5 to about 15 mM. In some aspects, the composition further comprises at least one buffer. Examples of buffers include, but are not limited to, at least one buffer selected from phosphate buffer, sodium phosphate monobasic, sodium phosphate dibasic, citrate, and borate. The at least one buffer is typically present in the composition at an amount ranging from about 1 mM to about 500 mM, such as about 2 mM to about 200 mM, about 50 mM to about 200 mM, about 5 mM to about 50 mM, about 7 mM to about 25 mM, about 9 mM to about 20 mM, or about 9 mM to about 15 mM.

In some cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises at least one salt. Examples of the at least one salt, include but are not limited to, at least one salt selected from sodium chloride, calcium chloride, and sodium sulfate. The at least one salt is typically present in an amount ranging from about 0.1 wt % to about 5 wt %, such as about 0.2 wt % to about 2.5 wt %, about 0.2 to about 0.85 wt %, about 0.2 wt % to about 0.8 wt %, about 0.3 wt % to about 0.75 wt %, based on weight of the composition.

In some aspects, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises at least one sugar. Examples of the at least one sugar include, but are not limited to, at least one sugar selected from dextrose, mannitol, and sucrose.

In some cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises at least one preservative. Examples of the at least one preservative include, but are not limited to, benzyl alcohol.

In some aspects, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises a flavoring agent.

In some aspects, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises a viscosity enhancer.

In some cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises glyceryl palmitostearate.

In some aspects, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, further comprises disintegrant. An example of the disintegrant includes, but is not limited to, croscarmellose sodium. The distintegrant is typically present in the composition in an amount ranging from about 1 wt % to about 5 wt %, based on weight of the composition.

Generally, the compositions, e.g., compositions described herein including at least one OCS and at least one of polyalkylene glycol, carboxymethyl cellulose or pharmaceutically acceptable salt thereof, and polyoxylglyceride as described herein, e.g., as described in the separately numbered aspects described herein, have an osmolality of from about 200 to about 2000 mmol/kg, such as about 270 to about 340 mmol/kg, e.g. about 270, 280, 290, 300, 310, 320, 330 or 340 mmol/kg, so that the composition (e.g., solution) is isotonic (iso-osmotic) with the blood, thereby decreasing pain upon injection, and precluding a need to add an isotonic agent. In some cases, the composition has an osmolality ranging from about 150 mmol/kg to about 3000 mmol/kg, such as about 200 mmol/kg to about 500 mmol/kg, about 270 mmol/kg to about 330 mmol/kg, about 280 mmol/kg to about 320 mmol/kg. However, high drug concentrations can be prepared and diluted with sterile water for IV infusion. Conversely, low drug concentration formulations may include an isotonic agent, such as sodium chloride or mannitol, to bring the isotonicity into the expected range for a parenteral dosage form.

In some cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, has a pH ranging from about 3 to about 10, such as about 3 to about 8, about 4 to about 8, about 6 to about 8, or about 7 to about 8.

In some aspects, when the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, is placed in a 1 mL syringe at 25° C. fitted with a 0.5 inch needle with a gauge of less than or equal to 21, such as a gauge of less than or equal to 22, 23, 24, 25, 26, or 27, and 10 lbs of force are applied, the composition is syringeable.

In some cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, is a ready-to-use suspension. In other cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, is a powder, e.g., lyophilized powder, e.g., for reconstitution prior to use. In some cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, is contained within a single-dose container. In other cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, is contained within a multi-dose container. In some cases, the composition, e.g., a composition described herein including at least one OCS and at least one of polyalkylene glycol, carboxymethyl cellulose or pharmaceutically acceptable salt thereof, and polyoxylglyceride as described herein, e.g., as described in the separately numbered aspects described herein, is contained within a bottle, vial, syringe, or capsule. Examples of capsule materials include, but are not limited to, gelatin and hydroxypropyl methylcellulose.

The compositions, e.g., compositions described herein including at least one OCS and at least one of polyalkylene glycol, carboxymethyl cellulose or pharmaceutically acceptable salt thereof, and polyoxylglyceride as described herein, e.g., as described in the separately numbered aspects described herein, are typically administered as liquid solutions, suspensions, emulsions, etc. or liquids suitable for injection and/or intravenous administration; various controlled release formulations; or as a cream or lotion; and the like. Solid forms suitable for administration, or for solution in, or suspension in, liquids prior to administration, are also encompassed.

Controlled release refers to the presentation or delivery of compounds in response to time, and commonly refers to time dependent release in oral dose formulations. Controlled release has several variants such as sustained release (where prolonged release is intended), pulsed release (bursts of drug are released at different times), delayed release (e.g. to target different regions of the gastrointestinal tract tract), etc. Controlled release formulations may prolong drug action and maintain drug levels within a desired therapeutic window to avoid potentially hazardous peaks in drug concentration following ingestion or injection, and to maximize therapeutic efficiency. In addition to pills, capsules and injectable drug carriers (that often have an additional release function), forms of controlled release medicines include gels, implants, devices and transdermal patches.

In some aspects, e.g. for the treatment of acute ALF, the compositions, e.g., compositions described herein including at least one OCS and at least one of polyalkylene glycol, carboxymethyl cellulose or pharmaceutically acceptable salt thereof, and polyoxylglyceride as described herein, e.g., as described in the separately numbered aspects described herein, are formulated for intravenous (IV) administration. In this case, the volume that is administered is generally greater than when other administration modes are used, e.g. about 50 to 1000 ml. In such formulations, the amount of OCS is still in the ranges described elsewhere herein.

In contrast, for compositions, e.g., compositions described herein including at least one OCS and at least one of polyalkylene glycol, carboxymethyl cellulose or pharmaceutically acceptable salt thereof, and polyoxylglyceride as described herein, e.g., as described in the separately numbered aspects described herein, that are used for intramuscular or intraperitoneal injection, the volume of liquid that is used to deliver a dose is typically much lower, e.g. from about 0.5 to about a 10 ml maximum.

Exemplary Diseases/Conditions that are Prevented and/or Treated

### Organ Dysfunction and Failure

In some aspects, methods for preventing and/or treating organ or organ system failure are provided. The methods include contacting an organ of interest (e.g. the liver) with a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein. If the organ of interest is within a patient (in vivo), then contact generally involves administering to the patient an amount of a composition that is effective or sufficient to prevent and/or treat dysfunction and/or failure of one or more organs or organ systems in the patient, e.g. is therapeutically effective to prevent or treat at least one symptom of organ dysfunction or failure exhibited by the patient. If an organ has already been harvested from a subject (i.e. from a donor), and is thus ex vivo, then contact generally involves contacting the organ with at least one composition, i.e. applying at least one composition to the organ, to preserve the organ, i.e. maintain the viability of the organ, and/or enhance maintenance of the organ, until it is transplanted.

Methods of preventing and/or treating conditions which lead to, cause or are caused by, or which are associated with organ dysfunction and failure are also described, e.g. prevention and/or treatment of inflammation, cell death (e.g. necrosis), consequences of ischemia, sepsis, and others. The methods involve administering, to a subject in need thereof, an amount of a composition, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, that is effective or sufficient to prevent and/or treat the condition.

As used herein, “organ” refers to a differentiated and/or relatively independent body structure comprising cells and tissues that performs some specialized function in the body of an organism. An “organ system” refers to two or more organs that work together in the execution of a body function. A hollow organ is an internal visceral organ (viscus) that forms a hollow tube or pouch, or that includes a cavity. Exemplary organs, the dysfunction or failure of which are prevented and/or treated by the administration of or contact with a composition of the present disclosure, include but are not limited to: heart, lungs, (e.g., lungs damaged by pulmonary fibrosis, e.g., associated with chronic asthma), liver, pancreas, kidneys, brain, intestines, colon, thyroid, etc. In some cases, the dysfunction or failure which is prevented and/or treated by the administration of the one or more OCS involves an organ other than the liver, for example heart, lungs, pancreas, kidneys, brain, intestines, colon, etc. In general, methods and compositions described herein that refer to “organs” should also be understood to include “organ systems”, unless otherwise specified.

“Organ dysfunction” denotes a condition or a state of health where an organ does not perform its expected function. Organ function represents the expected function of the respective organ within physiologic ranges. The person skilled in the art is aware of the respective function of an organ during medical examination. Organ dysfunction typically involves a clinical syndrome in which the development of progressive and potentially reversible physiological dysfunction in an organ, optionally in the absence of anatomic injuries.

“Organ failure” denotes an organ dysfunction to such a degree that normal homeostasis cannot be maintained without external clinical intervention.

“Acute organ dysfunction” refers to reduced organ function that occurs rapidly —in days or weeks (e.g., within 26 weeks, within 13 weeks, within 10 weeks, within 5 weeks, within 4 weeks, within 3 weeks, within 2 weeks, within 1 week, within 5 days, within 4 days, within 3 days, or within 2 days)—usually in a person who has no pre-existing disease.

“Acute organ failure” refers to loss of organ function that occurs rapidly—in days or weeks (e.g., within 26 weeks, within 13 weeks, within 10 weeks, within 5 weeks, within 4 weeks, within 3 weeks, within 2 weeks, within 1 week, within 5 days, within 4 days, within 3 days, or within 2 days)—usually in a person who has no pre-existing disease. For instance, the term “acute renal failure” means a rapid deterioration in renal function sufficient to result in accumulation of waste products in the body. Acute liver failure is discussed in more detail below.

As used herein, “ischemia” refers to a reduction in blood flow to an organ.

The terms “sepsis” and “septicemia” refer to a morbid condition resulting from the invasion of the bloodstream by microorganisms and their associated endotoxins.

“Endotoxin” refers to any harmful components of microbial cells such as lipopolysaccharides from the Gram-negative bacterial cell wall, peptidoglycans from Gram-positive bacteria, and mannan from fungal cell walls.

Those of skill in the art will recognize that one or more of organ dysfunction, organ failure, and/or one or more conditions which are precursors of organ dysfunction or failure may be comorbid, i.e. may be present in a subject or individual at the same time. For example, a subject may have active sepsis that results in organ failure. Thus, preventing and/or treating may overlap in that treating sepsis may, at the same time, prevent the occurrence of organ failure; or treating ischemia may prevent or treat inflammation that occurs following an ischemic event, that would lead to organ failure but for the administration of the present compositions.

In some aspects, the present disclosure thus provides compositions, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, and methods for preventing and/or treating the dysfunction and/or failure of one or more organs or organ systems in a subject in need thereof by administering a therapeutically effective amount of a composition as described herein. In some aspects, the organ and/or organ system dysfunction and/or failure is acute, e.g. acute liver failure.

The methods may include administering to the subject a therapeutically effective or sufficient amount of at least one composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein. The amount is sufficient to prevent and/or treat dysfunction of the organ(s) being treated, or to prevent and/or treat failure of the organ(s) being treated. In some aspects, the organ failure that is treated is Multiple Organ Dysfunction Syndrome (MODS). The methods generally include identifying or diagnosing subjects who are in need of such treatment, e.g. subjects that would benefit from such treatment e.g. due to being susceptible to organ dysfunction or failure, or already exhibiting at least one sign or symptom of organ dysfunction or failure. For example, the subject may be a member of a particular patient population such as those with disease resulting from acute insult (acute organ injury resulting from bacterial infection, severe burns, trauma, etc.), or chronic conditions (long-term exposure to organ-damaging medication), and/or from other causes which are discussed in more detail below.

The patient group(s) addressed by the present disclosure can also be defined as follows. The SOFA system was created in a consensus meeting of the European Society of Intensive Care Medicine in 1994 and further revised in 1996. The SOFA is a six-organ dysfunction/failure score measuring multiple organ failure daily. Each organ is graded from 0 (normal) to 4 (the most abnormal), providing a daily score of 0 to 24 points. The objective of the SOFA is to create a simple, reliable, and continuous score for clinical staff. Sequential assessment of organ dysfunction during the first few days of intensive care unit (ICU) or hospital admission is a good indicator of prognosis. Both the mean and highest SOFA scores are particularly useful predictors of outcome.

In one aspect, the patient group pursuant to the disclosure is one having as a lower threshold at least one SOFA score, being at 1 for at least one of the clinical criteria of respiration, or liver, or coagulation, or cardiovascular, or CNS, or renal on the day of admission to hospital or Intensive Care Unit (ICU). However, the patient may also have a score of 1 or 2, or more (e.g. 3 or 4) for at least one of the clinical criteria. Thus, said patient group is in need of therapeutic intervention pursuant to the present disclosure, and thus in need for prevention or reduction of organ dysfunction or organ failure, e.g. renal, liver, heart and/or lung organ dysfunction or organ failure.

Independent of the initial score, generally an increase in SOFA score during the first 48 hours in the ICU or in the hospital predicts a mortality rate of at least 50%. Thus, in another aspect, the patient group in need of therapeutic intervention for organ dysfunction/failure in accordance with present disclosure is characterized by having at least one SOFA score increased within the initial 48 hours after admission to hospital or ICU. In some aspects, the organ, organs or organ systems which is/are subject to failure comprise at least one member of the following: cardiovascular, respiratory, renal, haematological, neurological, gastrointestinal organs, hepatic organs, heart, liver, lungs, intestines, colon, kidneys, spleen, and brain.

Administration of the compositions of the present disclosure, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, may be applied for sake of prevention or reduction of organ dysfunction and organ failure, and thus may be, but is not necessarily intended for any methods of primary treatment or first line treatment to the chronic or acute disease or acute condition itself, which therefore can be termed as underlying disease(s). This means the present disclosure does not necessarily provide for a therapy of healing/curing e.g. infections, cancer, or tumors located in the respective organ, but for resuscitating the respective organ towards physiologic function. Accordingly, the therapy for a chronic or acute disease or acute condition of a patient within the scope of the present disclosure includes any kind of organ insufficiency, or poor organ function as an acute event.

Kidney Dysfunction and/or Failure

Kidney disease may be acute or chronic, or even acute-on-chronic renal failure as discussed below.

Acute kidney injury (AKI, previously called acute renal failure (ARF)) refers to an abrupt loss of kidney function that develops e.g. within about 7 days. AKI generally occurs because of damage to the kidney tissue caused by decreased renal blood flow (renal ischemia) from any cause e.g. low blood pressure, exposure to substances harmful to the kidney, an inflammatory process in the kidney, or an obstruction of the urinary tract which impedes the flow of urine. Causes of acute kidney injury include accidents, injuries, or complications from surgeries in which the kidneys are deprived of normal blood flow for extended periods of time. Heart-bypass surgery is an example of one such procedure. Drug overdoses, either accidental or from chemical overloads of drugs such as antibiotics or chemotherapy, may also cause the onset of acute kidney injury. AKI is diagnosed on the basis of characteristic laboratory findings, such as elevated blood urea nitrogen (BUN) and creatinine, or inability of the kidneys to produce sufficient amounts of urine (e.g. less than 400 mL per day in adults, less than 0.5 mL/kg/h in children or less than 1 mL/kg/h in infants). Thus, the present methods may include measuring or detecting one or more of these parameters in a subject and, if one or more or the measured parameters is positive and thus indicative of the presence of kidney malfunction developing within about 7 days, then diagnosing acute kidney injury and administering a composition as described herein to the subject, as described herein.

Chronic kidney disease (CKD) usually develops slowly and, initially, patients may show few symptoms. CKD can be the long term consequence of irreversible acute disease or part of a disease progression. CKD has numerous causes, including diabetes mellitus, long-term, uncontrolled hypertension, polycystic kidney disease, infectious diseases such as hantavirus, and certain genetic predisposition e.g. APOL1 gene variants. The present methods include administering a composition as described herein to a subject having CKD.

In some cases, the clinical criteria denoting the patient group(s) for kidney dysfunction/failure are as follows:
Patients at risk for kidney dysfunction/failure: GFR decrease >25%, serum creatinine increased 1.5 times or urine production of <0.5 ml/kg/hr for 6 hours
Patients with present kidney injury: GFR decrease >50%, doubling of creatinine or urine production <0.5 ml/kg/hr for 12 hours
Patients with kidney failure: GFR decrease >75%, tripling of creatinine or creatinine >355 μmol/l (with a rise of >44) (>4 mg/dl) or urine output below 0.3 ml/kg/hr for 24 hours
Patients with loss of kidney function: persistent acute kidney injury (AKI) or complete loss of kidney function for more than 4 weeks
End-stage renal disease: complete loss of kidney function for more than 3 months.

Contrast and enhancing dyes used for various types of imaging, especially iodine containing dyes, are also known to cause kidney damage, especially in susceptible populations such as the elderly, diabetics, those who already have some form of kidney impairment, etc. Contrast-induced nephropathy is defined as either a greater than 25% increase of serum creatinine or an absolute increase in serum creatinine of 0.5 mg/dL in the wake of administration of a dye e.g. for X-rays or computed tomography (CT) scans. Iodine containing dyes include but are not limited to iohexol, iodixanol and ioversol, as well as other ionic iodine dyes such as Diatrizoate (Hypaque 50), Metrizoate (Isopaque 370), and Ioxaglate (Hexabrix); and non-ionic contrast media such as Iopamidol (Isovue 370), Iohexol (Omnipaque 350), Ioxilan (Oxilan 350), Iopromide (Ultravist 370), and Iodixanol (Visipaque 320). The compositions described herein can prevent or lessen the impact of such dyes when administered, for example, before administration of the dye, and/or concomitantly with the dye and/or after dye administration to maintain kidney values at a normal level in spite of exposure to the dye, or to facilitate or speed the return of those values to safe, normal ranges after dye administration.

Liver Dysfunction and/or Failure

An exemplary aspect of the present disclosure involves the treatment of acute liver failure, especially acute liver failure caused by necrosis. Acute liver failure involves the rapid development of hepatocellular dysfunction, specifically coagulopathy and mental status changes (encephalopathy) in a patient without known prior liver disease. This malady embraces a number of conditions whose common thread is severe injury of hepatocytes and/or massive necrosis e.g. loss of function of 80-90% of liver cells. Loss of hepatocyte function sets in motion a multiorgan response characterized by the rapid appearance of severe complications soon after the first signs of liver disease (such as jaundice). Complications include hepatic encephalopathy and impaired protein synthesis, e.g. as measured by the levels of serum albumin and the prothrombin time in the blood. Up to now, treatment options for acute liver failure have been limited and death often occurs suddenly, even after the liver has begun to recover from the original damage.

The diagnosis of acute liver failure (i.e. the identification of a subject experiencing acute liver failure and who could benefit from the practice of the present methods) is generally based on physical exam, laboratory findings, patient history, and past medical history to establish, for example, mental status changes, coagulopathy, rapidity of onset, and absence of known prior liver disease. The exact definition of “rapid” depends on the particular convention that is used. Different sub-divisions exist which are based on the time from onset of first hepatic symptoms to onset of encephalopathy. One scheme defines “acute hepatic failure” as the development of encephalopathy within 26 weeks of the onset of any hepatic symptoms. This is sub-divided into “fulminant hepatic failure”, which requires onset of encephalopathy within 8 weeks, and “subfulminant”, which describes onset of encephalopathy after 8 weeks but before 26 weeks. Another scheme defines “hyperacute” liver failure as onset within 7 days, “acute” liver failure as onset between 7 and 28 days, and “subacute” liver failure as onset between 28 days and 24 weeks. Subjects identified as experiencing acute liver failure by any of these criteria may be treated by the methods described herein.

In some cases, the patient group for liver dysfunction/failure is characterized by a lower threshold of Bilirubin of >1.2 mg/dL, such as >1.9 mg/dL, or >5.9 mg/dL. Acute liver failure has many potential causes and subjects identified as experiencing acute liver failure for any reason can be treated by the methods described herein. Possible causes include:

Acetaminophen (APAP). Taking too much acetaminophen (paracetamol, Tylenol®, others) is the most common cause of acute liver failure in the United States. Acute liver failure can occur if a single very large dose of APAP is taken all at once, or it can occur if higher-than-recommended doses are taken every day for several days. People with chronic liver disease are especially vulnerable, as are the elderly, the very young, etc. In such subjects, an APAP “overdose” may be a dose that would be a safe or normal dose for a person that does not have chronic liver disease or is not elderly or very young. This aspect of the disclosure is discussed in detail below.
Prescription medications. Some prescription medications, including antibiotics, nonsteroidal anti-inflammatory drugs and anticonvulsants, can cause acute liver failure. Herbal supplements. Herbal drugs and supplements, including kava, ephedra, skullcap and pennyroyal, have been linked to acute liver failure.
Hepatitis and other viruses. Hepatitis A, hepatitis B and hepatitis E can cause acute liver failure. Other viruses that can cause acute liver failure include Epstein-Barr virus, cytomegalovirus and herpes simplex virus.
Toxins. Toxins that can cause acute liver failure include the poisonous wild mushroom Amanita phalloides, which is sometimes mistaken for edible species.
Autoimmune disease. Liver failure can be caused by autoimmune hepatitis, a disease in which the immune system attacks liver cells, causing inflammation and injury.
Diseases of the veins in the liver. Vascular diseases, such as Budd-Chiari syndrome, can cause blockages to form in the veins of the liver and lead to acute liver failure.
Metabolic disease. Rare metabolic diseases, such as Wilson's disease and acute fatty liver of pregnancy, can cause acute liver failure.
Cancer. Cancer that begins in the liver or cancer that spreads to the liver from other locations in the body can cause acute liver failure.
Other. Other causes include idiosyncratic reactions to medication (e.g. tetracycline, troglitazone), excessive alcohol intake (severe alcoholic hepatitis), Reye syndrome (acute liver failure in a child with a viral infection e.g. chickenpox in which aspirin may play a role; and others. Many cases of acute liver failure have no apparent cause.

In addition, various symptoms of liver toxicity may be prevented and/or treated by the methods and compositions of the present disclosure, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, prior to the development of full-blown ALF. Exemplary symptoms include but are not limited to: cerebral edema and encephalopathy (which may lead to hepatic encephalopathy, coma, brain herniation, etc.); coagulopathy (e.g. prolongation in prothrombin time, platelet dysfunction, thrombocytopenia, intracerebral bleeding, etc.); renal failure (e.g. due to original insult such as APAP overdose resulting in acute tubular necrosis, or from hyperdynamic circulation leading to hepatorenal syndrome or functional renal failure); inflammation and infection (e.g. systemic inflammatory syndrome, which can lead to sepsis and multi-organ failure irrespective of the presence or absence of infection); various metabolic derangements such as hyponatremia, hypoglycemia, hypokalemia, hypophosphatemia, metabolic alkalosis, and lactic acidosis (occurring predominantly in acetaminophen overdose); hemodynamic and cardio-respiratory compromise (e.g. hypotension, decrease in tissue oxygen uptake, tissue hypoxia and lactic acidosis); pulmonary complications (e.g. acute respiratory distress syndrome (ARDS), with or without sepsis, pulmonary haemorrhage, pleural effusions, atelectasis, and intrapulmonary shunts, etc.); late pregnancy complications, for which early clinical manifestations of ALF include hypodynamia, decrease in appetite, dark amber urine, deep jaundice, nausea, vomiting, and abdominal distention, etc. Subjects exhibiting one or more of these symptoms or conditions may benefit from the administration of at least one OCS.

### Acute Liver Failure Due to APAP Toxicity

In some aspects, the present disclosure provides methods and compositions, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, for preventing and/or treating APAP associated toxicity and symptoms associated with or characteristic thereof, especially liver injury or ALF as discussed above. APAP toxicity is one of the most common causes of poisoning worldwide and in the United States and the United Kingdom it is the most common cause of acute liver failure. Many individuals with APAP toxicity may have no symptoms at all in the first 24 hours following overdose. Others may initially have nonspecific complaints such as vague abdominal pain and nausea. With progressive disease, signs of liver failure usually develop; these include low blood sugar, low blood pH, easy bleeding, and hepatic encephalopathy. Damage to the liver, or hepatotoxicity, results not from APAP itself, but from one of its metabolites, N-acetyl-p-benzoquinoneimine (NAPQI), also known as N-acetylimidoquinone. NAPQI depletes the liver's natural antioxidant glutathione and directly damages cells in the liver, leading to liver failure. Risk factors for APAP toxicity include excessive chronic alcohol intake, fasting or anorexia nervosa, and the use of certain drugs such as isoniazid.

Methods to prevent or treat ALF in a subject in need thereof, especially liver dysfunction and/or acute liver failure associated with APAP toxicity, are described in this disclosure. The methods may include administering a composition as described herein prior to administration of APAP, and/or concomitantly with administration of APAP, and/or after administration of APAP, to prevent and/or treat APAP toxicity.

### Pancreas Dysfunction and Failure

The pancreas is a glandular organ that functions in the digestive system and endocrine system of vertebrates. It produces several important hormones, including insulin, glucagon, somatostatin, and pancreatic polypeptide, and also secretes pancreatic juice containing digestive enzymes that assist digestion and absorption of nutrients in the small intestine. Inflammation of the pancreas (pancreatitis) has several causes and typically requires immediate treatment. It may be acute, beginning suddenly and lasting a few days, or chronic, occurring over many years. Eighty percent of cases of pancreatitis are caused by alcohol or gallstones, with gallstones being the single most common etiology of acute pancreatitis and alcohol being the single most common etiology of chronic pancreatitis. Severe pancreatitis is associated with organ failure, necrosis, infected necrosis, pseudocyst and abscess, having mortality rates around 2-9%, and higher where necrosis has occurred. Severe pancreatitis is diagnosed if at least three of the following are true: patient age is greater than 55 years; blood PO2 oxygen is less than 60 mm Hg or 7.9 kP; white blood cells >15,000 WBCs per microliter (mcL); calcium <2 mmol/L; urea >16 mmol/L; lactate dehydrogenase (LDH) >600 iu/L; aspartate transaminase (AST) >200 iu/L; albumin <32 g/L; and glucose >10 mmol/L.

An aspect of the present disclosure is the treatment of pancreatic dysfunction and/or failure by administering a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, to a patient in need thereof. Suitable patients or patient populations are identified, by a skilled medical practitioner, as exhibiting at least one of the symptoms or criteria listed above.

Heart Dysfunction and/or Failure

Heart failure (HF), often used to mean chronic heart failure (CHF), occurs when the heart is unable to pump sufficiently to maintain blood flow to meet the needs of the body. The terms congestive heart failure (CHF) or congestive cardiac failure (CCF) are often used interchangeably with chronic heart failure. Symptoms commonly include shortness of breath (especially with exercise, when lying down, and at night while sleeping), excessive tiredness, and leg swelling. Common causes of heart failure include coronary artery disease including a previous myocardial infarction (heart attack), high blood pressure, atrial fibrillation, valvular heart disease, and cardiomyopathy. Heart failure is distinct from myocardial infarction, in which part of the heart muscle dies, and cardiac arrest, in which blood flow stops altogether.

Heart failure is typically diagnosed based on the history of the symptoms and a physical examination with confirmation by echocardiography, blood tests, and/or chest radiography. Echocardiography uses ultrasound to determine the stroke volume (SV, the amount of blood in the heart that exits the ventricles with each beat), the end-diastolic volume (EDV, the total amount of blood at the end of diastole), and the SV in proportion to the EDV, a value known as the ejection fraction (EF). Abnormalities in one or more of these may indicate or confirm heart dysfunction and/or failure. An electrocardiogram (ECG/EKG) is used to identify arrhythmias, ischemic heart disease, right and left ventricular hypertrophy, and presence of conduction delay or abnormalities (e.g. left bundle branch block). Abnormalities in one or more of these may also indicate or confirm heart dysfunction and/or failure. Blood tests routinely performed to diagnose or confirm heart dysfunction/failure include electrolytes (sodium, potassium), measures of renal function, liver function tests, thyroid function tests, a complete blood count, and often C-reactive protein if infection is suspected. Abnormalities in one or more of these may also indicate or confirm the presence of heart dysfunction and/or failure. An elevated B-type natriuretic peptide (BNP) is a specific test indicative of heart failure. If myocardial infarction is suspected, various cardiac markers may be tested, including but not limited to troponin creatine kinase (CK)-MB (an isoform of creatine kinase); lactate dehydrogenase; aspartate transaminase (AST) (also referred to as aspartate aminotransferase); myoglobin; ischemia-modified albumin (IMA); pro-brain natriuretic peptide; glycogen phosphorylase isoenzyme BB, etc. Abnormal levels of one or more of these (usually abnormally high levels) are considered as identifying a subject in need of treatment for cardiac dysfunction or failure.

Heart failure may also occur as a side effect and/or in the aftermath of chemotherapy, e.g. chemotherapy received as treatment for cancer such as breast cancer. The administration of a composition as described herein to a patient receiving or who has already received chemotherapy may prevent unwanted damage to heart (and other organs, organ systems, tissues and cells) during or after cancer chemotherapy. In other words, the composition as described herein is used as a protective agent for deleterious effects of chemotherapy.

A subject who is confirmed to have or suspected of having cardiac dysfunction or failure is treated by administration of a therapeutically effective amount of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, the amount being sufficient to prevent symptoms of heart dysfunction or failure, or to ameliorate symptoms of heart dysfunction or failure, e.g. to at least partially restore heart function to normal or near normal, and/or to prevent further deterioration of heart function and health of the patient.

Brain Dysfunction and/or Failure

Brain dysfunction and/or failure (i.e. organic brain syndrome “OBS”) is a general term that describes decreased mental function due to a medical disease other than a psychiatric illness. Causes include but are not limited to brain injury caused by trauma; bleeding into the brain (intracerebral hemorrhage); bleeding into the space around the brain (subarachnoid hemorrhage); blood clot inside the skull causing pressure on brain (subdural hematoma); concussion; various breathing conditions such as low oxygen in the body (hypoxia) and high carbon dioxide levels in the body (hypercapnia); various cardiovascular disorders, e.g. dementia due to many strokes or multi-infarct dementia, heart infections (endocarditis, myocarditis), stroke (e.g. spontaneous stroke) and transient ischemic attack (TIA) or so-called “ministrokes”; or due to various degenerative disorders such as Alzheimer disease, Creutzfeldt-Jacob disease, diffuse Lewy Body disease, Huntington disease, multiple sclerosis, normal pressure hydrocephalus, Parkinson disease and Pick disease; dementia due to metabolic causes such as kidney, liver, or thyroid disease and/or vitamin deficiency (B1, B12, or folate); as well as drug and alcohol-related conditions e.g. alcohol withdrawal state, intoxication from drug or alcohol use, Wernicke-Korsakoff syndrome (a long-term effect of excessive alcohol consumption or malnutrition), and withdrawal from drugs (especially sedative-hypnotics and corticosteroids); and sudden onset (acute) or long-term (chronic) infections e.g. septicemia, encephalitis, meningitis, prion infections, and late-stage syphilis; as well as complications of cancer or cancer treatment. Symptoms of OBS include agitation, confusion; long-term loss of brain function (dementia), and severe, short-term loss of brain function (delirium), as well as impacts on the autonomic nervous system which controls e.g. breathing. Diagnosis or confirmation of the presence of OBS is determined by detecting or measuring various methodology such as blood tests, electroencephalogram (EEG), head CT scan, head MRI and/or lumbar puncture, for which normal values typically range as follows: pressure: 70-180 mm Hg; cerebral spinal fluid (CSF) appearance: clear, colorless; CSF total protein: 15-60 mg/100 mL; gamma globulin: 3-12% of the total protein; CSF glucose: 50-80 mg/100 mL (or greater than ⅔ of blood sugar level); CSF cell count: 0-5 white blood cells (all mononuclear), and no red blood cells; and CSF chloride: 110-125 mEq/L.

If one or more of these tests or analyses or indicia are abnormal, the subject is generally considered as susceptible to or already suffering from OBS. A subject who is confirmed to have or suspected of having OBS (either early stage or advanced) is treated by administration of a therapeutically effective amount of a composition comprising at least one OCS as described herein (e.g. 25HC3S), e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, the amount being sufficient to prevent symptoms of OBS, or to ameliorate symptoms of OBS, e.g. to at least partially restore brain function to normal or near normal, and/or to prevent further deterioration of brain function and health of the patient.

Organ Dysfunction and/or Failure Due to Trauma

In some aspects, the organ dysfunction/failure is due to trauma. Examples of trauma injuries include but are not limited to: wounds resulting from vehicular accidents; gunshot wounds (both accidental during hunting associated activities, and intentionally inflicted such as those associated with criminal activity or war); blunt trauma or blunt injury e.g. non-penetrating blunt force trauma such as physical trauma to a body part e.g. by impact, injury or physical attack; etc. Examples of blunt trauma include but are not limited to: concussion, e.g. concussion suffered by athletes or by persons involved in accidents, falls, etc., and blunt trauma suffered as the result of an encounter with a projectile such as a falling object, and others.

Individuals who are susceptible to such blunt trauma (e.g. athletes, the elderly) may benefit from prophylactic administration of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, and if blunt trauma such as a concussion is diagnosed in a subject, the subject will benefit by administration as soon as possible after the injury is suspected or confirmed.

Prevention and/or Treatment of Conditions Caused by Ischemia

Ischemia refers to an insufficient supply of blood to a tissue or organ, causing a shortage of oxygen and glucose needed for cellular metabolism and to keep tissue alive. Hypoxia (also known as hypoxiation or anoxemia) is caused by ischemia and refers to the condition in which the body or a region of the body is deprived of adequate oxygen supply. Ischemia results in tissue damage in a process known as the ischemic cascade. Damage is largely the result of the build-up of metabolic waste products, the inability to maintain cell membranes, mitochondrial damage, and eventual leakage of autolyzing proteolytic enzymes into the cell and surrounding tissues. Ensuing inflammation also damages cells and tissues. Without immediate intervention, ischemia may progress quickly to tissue necrosis, and ultimately to, for example, organ dysfunction or failure.

In addition, restoration of blood supply to ischemic tissues can cause additional damage known as reperfusion injury. Reperfusion injury can be more damaging than the initial ischemia. Reintroduction of blood flow brings oxygen back to the tissues, causing a greater production of free radicals and reactive oxygen species that damage cells. It also brings more calcium ions to the tissues, which may cause calcium overloading and can result in potentially fatal cardiac arrhythmias, and which may accelerate cellular self-destruction. The restored blood flow may also exaggerate the inflammation response of damaged tissues, causing white blood cells to destroy damaged but still viable cells.

The present disclosure provides methods and compositions for preventing and/or treating the untoward effects or outcomes of ischemia, including ischemia/reperfusion injury, in a subject in need thereof. The methods generally comprise administering a therapeutically effective amount of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, sufficient to prevent or treat symptoms of ischemia and/or ischemia/reperfusion. The methods may also include identifying or diagnosing a subject who will experience, or is experiencing or who has experienced ischemia and/or ischemia/reperfusion. The ischemia and/or ischemia/reperfusion may be due to a disease process (e.g. atherosclerosis, a blood clot, etc.), or due to an accident (e.g. severing of an artery or other blood conduit), or may be intentional (planned), e.g. as occurs during some heart or other surgeries in order to temporarily stop blood flow to a defined or circumscribed region of the body.

Types of ischemia that are relevant to the methods described herein include but are not limited to:

Cardiac ischemia, e.g., myocardial ischemia, occurring when the heart muscle, or myocardium, receives insufficient blood flow. This most frequently results from atherosclerosis, which is the long-term accumulation of cholesterol-rich plaques in coronary arteries.
Bowel ischemia: Both large and small bowel can be affected by ischemic injury. Ischemic injury of the large intestine may result in an inflammatory process known as ischemic colitis and also as a result of surgery and adhesion development. Ischemia of the small bowel is called mesenteric ischemia.
Brain ischemia is insufficient blood flow to the brain, and can be acute (i.e., rapid) or chronic (i.e., long-lasting). Acute ischemic stroke is a neurologic emergency that may be reversible if treated rapidly. Chronic ischemia of the brain may result in a form of dementia called vascular dementia. A brief episode of ischemia affecting the brain is called a transient ischemic attack (TIA), often erroneously referred to as a “mini-stroke”.
Limb ischemia: Lack of blood flow to a limb results in acute limb ischemia.
Cutaneous ischemia refers to reduced blood flow to the skin layers, which may result in mottling or uneven, patchy discoloration of the skin, and may lead to the development of cyanosis, or other conditions such as pressures sores (e.g. decubitus ulcers, bedsores, etc.).
Reversible ischemia refers to a condition which results in a lack of blood flow to a particular organ which can be reversed through use of medications or surgery. It most often refers to hindered blood flow to the heart muscle, but it can refer to an obstruction blocking any organ in the body, including the brain. Whether or not a case of ischemia can be reversed will depend on the underlying cause. Plaque buildup in the arteries, weakened arteries, low blood pressure, blood clots, and unusual heart rhythms can all be causes of reversible ischemia.
Apical ischemia refers to lack of blood flow to the apex or bottom tip of the heart.
Mesenteric ischemia refers to inflammation and injury of the small intestine occurs due to inadequate blood supply. Causes of the reduced blood flow can include changes in the systemic circulation (e.g. low blood pressure) or local factors such as constriction of blood vessels or a blood clot.
Ischemia of various organs, including but not limited to liver (hepatic ischemia), kidney, intestines, etc.

Ischemia, ischemia/reperfusion may also be causally related to inflammation and organ dysfunction/failure. For example, cerebral (brain) ischemia is typically accompanied by a marked inflammatory reaction that is initiated by ischemia-induced expression of cytokines, adhesion molecules, and other inflammatory mediators, including prostanoids and nitric oxide. It is known that interventions aimed at attenuating such inflammation reduce the progression of brain damage that occurs e.g. during the late stages of cerebral ischemia. In addition, the most frequent cause of intrarenal (kidney) failure (ARF) is transient or prolonged renal hypoperfusion (ischemia).

Other types of ischemia, the effects of which can be treated or prevented as described herein, include but are not limited to: ischemic stroke, small vessel ischemia, ischemia/reperfusion injuries, etc.

Diagnosis of ischemia is generally carried out by identifying one or more symptoms of malfunction in the particular organ or organ system or tissue or cell that is affected. Thus, symptoms include those listed herein for dysfunction/failure of individual organs, plus documentation of ischemia per se, such as by noting the history of the patient (e.g. known occlusion, blockage or severance of an artery that otherwise supplies blood to the organ or tissue, imaging which shows or is consistent with such observations, etc.).

If one or more suitable tests or analyses or indicia are abnormal, the subject is generally considered as susceptible to or already suffering from ischemia. A subject who is confirmed to have or suspected of having ischemia (or is known to be undergoing future planned ischemia, e.g. during a surgical procedure) may be treated by administration of a therapeutically effective amount of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, the amount being sufficient to prevent symptoms of ischemia and/or ischemia-reperfusion injury, or to ameliorate symptoms of ischemia and/or ischemia-reperfusion injury, e.g. to at least partially restore organ or tissue function to normal or near normal when blood flow is reestablished, and/or to prevent further deterioration of organ or tissue function and health of the patient.

Prevention and/or Treatment of Effects of Unwanted Cell Death

Active, regulated cell death is referred to as “programmed cell-death” or “PCD” and is a regulated process mediated by intracellular pathways. While PCD is generally beneficial to an organism, aberrations in signaling or the presence of overwhelming stresses on the cell may cause undesirable PCD to occur. The forms of PCD include apoptosis, the initiation of controlled intracellular signaling in response to a stress, which brings about cell suicide; and necroptosis, a form of PCD that serves as a backup to apoptosis, e.g. when the apoptosis signaling is blocked by endogenous or exogenous factors such as viruses or mutations.

In contrast to PCD, necrosis refers to unregulated, passive cell death which results in the harmful, premature death of cells in living tissue. Necrosis is typically caused by factors external to the cell or tissue, such as infection, toxins, trauma, ischemia, etc. Without being bound by theory, it is believed that necrosis involves the loss of cell membrane integrity and an uncontrolled release of products of cell death into the intracellular space, thereby initiating an inflammatory response in the surrounding tissue which prevents nearby phagocytes from locating and eliminating the dead cells by phagocytosis. While surgical removal of necrotic tissue can halt the spread of necrosis, in some cases surgical intervention is not possible or practical e.g. when internal tissues or organs are involved. Thus, necrosis of internal organs often leads to dangerous and often deadly organ dysfunction and/or failure.

The present disclosure provides methods and compositions for preventing and/or treating the effects of unwanted cell death in a subject in need thereof, especially unwanted apoptosis and necrosis associated with organ dysfunction and/or organ failure. The cell death may result from or be associated with unwanted PCD (e.g. unwanted or deleterious apoptosis, autophagy, or necroptosis) or with necrosis, which is unwanted by definition; and/or combinations of these. The methods comprise administering a therapeutically effective amount of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, the amount being sufficient to prevent unwanted cell death from occurring, or to treat the effects of unwanted cell death that has already occurred in a subject.

Unwanted or deleterious cell death via apoptosis occurs, for example, in the aftermath of ischemia and in Alzheimer's disease. Unwanted apoptosis is extremely harmful, causing extensive tissue damage.

Types of necrosis that may be prevented and/or treated by the methods described herein include but are not limited to:

Aseptic necrosis is necrosis without infection, usually in the head of the femur after traumatic hip dislocation.
Acute tubular necrosis refers to acute renal failure with mild to severe damage or necrosis of tubule cells, usually secondary to either nephrotoxicity, ischemia after major surgery, trauma (crush syndrome), severe hypovolemia, sepsis, or burns.
Avascular necrosis is the consequence of temporary or permanent cessation of blood flow to the bones. The absence of blood causes the bone tissue to die, resulting in fracture or collapse of the entire bone.
Balser's fatty necrosis is gangrenous pancreatitis with omental bursitis and disseminated patches of necrosis of fatty tissues.
Bridging necrosis is necrosis of the septa of confluent necrosis bridging adjacent central veins of hepatic lobules and portal triads characteristic of subacute hepatic necrosis.
Caseous or “cheesy” necrosis is necrosis in which the tissue is soft, dry, and cottage cheese-like, most often seen in tuberculosis and syphilis; in contrast to moist necrosis in which the dead tissue is wet and soft.
Central necrosis is necrosis affecting the central portion of an affected bone, cell or lobule of the liver.
Coagulation necrosis refers to necrosis of a portion of an organ or tissue, with formation of fibrous infarcts, the protoplasm of the cells becoming fixed and opaque by coagulation of the protein elements, the cellular outline persisting for a long time.
Colliquative or liquefaction necrosis is that in which the necrotic material becomes softened and liquefied.
Contraction band necrosis refers to a cardiac lesion characterized by hypercontracted myofibrils and contraction bands, and mitochondrial damage caused by calcium influx into dying cells resulting in arrest of the cells in the contracted state.
Fat necrosis is that in which the neutral fats in adipose tissue are broken down into fatty acids and glycerol, usually affecting the pancreas and peripancreatic fat in acute hemorrhagic pancreatitis.
Gangrenous necrosis is that in which ischemia combined with bacterial action causes putrefaction to set in. “Gangrene” includes dry gangrene, wet gangrene, gas gangrene, internal gangrene and necrotizing fasciitis.
Gingival necrosis refers to the death and degeneration of the cells and other structural elements of the gingivae (e.g., necrotizing ulcerative gingivitis).
Interdental necrosis is a progressive disease that destroys the tissue of the papillae and creates interdental craters. Advanced interdental necrosis leads to a loss of periodontal attachment.
Ischemic necrosis refers to death and disintegration of a tissue resulting from interference with its blood supply, thus depriving the tissues of access to substances necessary for metabolic sustenance.
Macular degeneration: Macular degeneration (both wet and dry forms) occurs when the small central portion of the retina, known as the macula, deteriorates. Because the disease develops as a person ages, it is often referred to as age-related macular degeneration (AMD).
Massive hepatic necrosis refers to massive, usually fatal, necrosis of the liver, a rare complication of viral hepatitis (fulminant hepatitis) that may also result from exposure to hepatotoxins or from drug hypersensitivity.
Phosphorus necrosis is necrosis of the jaw bone due to exposure to phosphorus.

Postpartum pituitary necrosis refers to necrosis of the pituitary during the postpartum period, often associated with shock and excessive uterine bleeding during delivery, and leading to variable patterns of hypopituitarism.

Radiation necrosis is the death of tissue caused by radiation.
Selective myocardial cell necrosis refers to myofibrillar degeneration.
Zenker's necrosis refers to hyaline degeneration and necrosis of striated muscle; also called Zenker's degeneration.

Such unwanted or pathological cell death may be prevented or treated by contacting affected cells with a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, in an amount sufficient to prevent or treat death of the cells, and/or to prevent the spread of cell death signaling to adjacent cells. Candidate cells for treatment, or organs containing candidate cells for treatment, are identified by any of several known techniques, e.g. by observation of overt effects of cell death (tissue breakdown, liquefaction, odor, etc.), detecting release of lactate dehydrogenase (LDH), by various scans such as tomography or nuclear magnetic resonance, by detecting the presence of causative bacteria (e.g. using PCR), using antibodies, etc.

Prevention and/or Treatment of Symptoms Related to or Caused by Sepsis (Inflammatory Response Syndrome, or Sirs)

Sepsis is a potentially life-threatening whole-body inflammation caused by a serious infection which triggers an immune response. The infection is typically caused by bacteria, but can also be due to fungi, viruses, or parasites in the blood, urinary tract, lungs, skin, or other tissues. Unfortunately, symptoms can continue even after the infection is gone. Severe sepsis is sepsis causing poor organ function or insufficient blood flow as evidenced e.g. by low blood pressure, high blood lactate, and/or low urine output. In fact, sepsis is considered to fall within a continuum from infection to multiple organ dysfunction syndrome (MODS). Septic shock is low blood pressure due to sepsis that does not improve after reasonable amounts of intravenous fluids are given.

Up to now, sepsis was typically treated with intravenous fluids and antibiotics, often in an intensive care unit. Various medications and other interventions may be used, e.g. mechanical ventilation, dialysis, and oxygen saturation may also be used. Outcomes depend on the severity of disease with the risk of death from sepsis being as high as 30%, severe sepsis as high as 50%, and septic shock as high as 80%. Provided herein are methods of preventing or treating sepsis by administering to a subject or patient in need thereof, a therapeutically effective amount of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein. For instance, the present disclosure includes the treatment of mammalian endotoxemia and septicemia and renal and mesenteric vasoconstriction that is induced by catecholamines that are used to treat endotoxemia and septic shock. The term “endotoxemia′ refers to the presence of microbial endotoxins in the bloodstream. Subjects inflicted with endotoxemia usually also have septicemia. The present disclosure includes a method for treating septicemia/endotoxemia. The present disclosure also includes a method for treating acute renal failure caused by septicemia/endotoxemia by administering an effective amount of a composition described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein.

Further, the present disclosure includes a method for treating renal vasoconstriction caused by septicemia/endotoxemia. Still further, the present disclosure provides a method for attenuating catechol amine-induced renal and mesenteric vasoconstriction. Yet further, the present disclosure includes a method to prevent damage to a patient's intestines and kidney due to the effects of endotoxin and/or vasopressor agents. Sepsis is associated with mitochondrial dysfunction, which leads to impaired oxygen consumption and may lead to sepsis-induced multiple organ failure. This holds especially true for raised tissue oxygen tensions in septic patients, suggesting reduced ability of the organs to use oxygen. Because ATP production by mitochondrial oxidative phosphorylation accounts for more than 90% of total oxygen consumption, mitochondrial dysfunction may directly results in organ failure, possibly due to nitric oxide, which is known to inhibit mitochondrial respiration in vitro and is produced in excess in sepsis. Therefore, in a specific embodiment of the present disclosure, the compositions described herein, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, are used in methods of prevention for organ dysfunction and failure in Systemic Inflammatory Response-Syndrome (SIRS), sepsis, severe sepsis, and septic shock patients.

The methods may include identifying a suitable patient in need of such treatment, e.g. by detecting or measuring at least one symptom of sepsis, e.g. abnormal temperature (body temperature above 101 F (38.3 C, “fever”) or below 96.8 F (36 C), increased heart rate, increased breathing rate, probable or confirmed infection, and possibly confusion. Patients with severe sepsis exhibit at least one of the following signs and symptoms, which indicate an organ may be failing: significantly decreased urine output, abrupt change in mental status, decrease in platelet count, difficulty breathing, abnormal heart pumping function, and abdominal pain. A diagnosis of septic shock is generally based on observing the signs and symptoms of severe sepsis plus measuring extremely low blood pressure that does not adequately respond to simple fluid replacement. In some cases, a subject may be a candidate for prophylactic or therapeutic treatment of sepsis based on cough/sputum/chest pain; abdominal pain/distension/diarrhea; line infection; endocarditis; dysuria; headache with neck stiffness; cellulitis/wound/joint infection; and/or positive microbiology for any infection. In other cases, a subject may be a candidate for prophylactic or therapeutic treatment with OCS of severe sepsis based on a diagnosis of sepsis and at least one clinical suspicion of any organ dysfunction selected from: blood pressure systolic <90/mean; <65 mm HG; lactate >2 mmol/L; Bilirubin >34 μmon; urine output <0.5 mL/kg/h for 2 h; creatinine >177 μmon; platelets <100×10⁹/L; and SpO₂>90% unless O₂ given. In some cases, a subject may be a candidate for prophylactic or therapeutic treatment of septic shock if there is refractory hypotension that does not respond to treatment and intravenous systemic fluid administration alone is insufficient to maintain a patient's blood pressure from becoming hypotensive. Patients with a diagnosis of (exhibiting signs of) early sepsis, severe sepsis or septic shock are candidates for treatment with a composition as described herein, e.g. by administration of a therapeutically effective amount of the composition. The amount administered may be sufficient to prevent symptoms of sepsis from developing or continuing, or to at least lessen the impact of symptoms of sepsis.

### Hyperlipidemia

In some aspects, the subjects treated by the compositions and methods described herein, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, have symptoms of and/or have been diagnosed with high levels of lipids i.e. hyperlipidemia. Hyperlipidemias are also classified according to which types of lipids are elevated, that is hypercholesterolemia, hypertriglyceridemia or both in combined hyperlipidemia. Elevated levels of lipoprotein(a) is also included. Hypercholestolemia generally refers to cholesterol levels in serum in the range of about 200 mg/dl or more. Hypertriglyceridemia is characterized, for example as borderline (150 to 199 mg per dL), or high (200 to 499 mg per dL) or very high (500 mg per dL or greater). These conditions are treated by the compositions described herein, as are diseases or conditions associated therewith e.g. atherosclerosis, heart disease, stroke, Alzheimer's, gallstone diseases, cholestatic liver diseases, pancreatitis, etc. The compositions disclosed herein, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, are used to lower cholesterol and/or lipid levels in the subject. By “lowering cholesterol levels” we mean that the level of free serum cholesterol in a patient is decreased by at least about 10% to 30%, and preferably at least about 30 to 50%, and more preferably at least about 50 to 70%, and most preferably at least about 70 to about 100%, or more, in comparison to the level of cholesterol in the subject prior to administration of the composition. Alternatively, the extent of the decrease may be determined by comparison to a similar untreated control population to whom the compound is not administered. Those of skill in the art are familiar with such determinations, e.g. the use of controls, or the measurement of cholesterol levels in the blood before and after administration of an agent that lowers cholesterol and/or lipids.

In some aspects, the disease or condition that is prevented or treated is or is caused by hyperlipidemia. By “hyperlipidemia” we mean a condition of abnormally elevated levels of any or all lipids and/or lipoproteins in the blood. Hyperlipidemia includes both primary and secondary subtypes, with primary hyperlipidemia usually being due to genetic causes (such as a mutation in a receptor protein), and secondary hyperlipidemia arising from other underlying causes such as diabetes. Lipids and lipid composites that may be elevated in a subject and lowered by the treatments described herein include but are not limited to chylomicrons, very low-density lipoproteins, intermediate-density lipoproteins, low-density lipoproteins (LDLs) and high-density lipoproteins (HDLs). In particular, elevated cholesterol (hypercholesteremia) and triglycerides (hypertriglyceridemia) are known to be risk factors for blood vessel and cardiovascular disease due to their influence on atherosclerosis. Lipid elevation may also predispose a subject to other conditions such as acute pancreatitis. The methods of the disclosure thus may also be used in the treatment or prophylaxis (e.g. prophylactic treatment) of conditions that are or are associated with elevated lipids. Such conditions include, for example, but are not limited to: hyperlipidemia, hypercholesterolemia, hypertriglyceridemia, fatty liver (hepatic steatosis), metabolic syndrome cardiovascular diseases, coronary heart disease, atherosclerosis (i.e. arteriosclerotic vascular disease or ASVD) and associated maladies, acute pancreatitis, various metabolic disorders, such as insulin resistance syndrome, diabetes, polycystic ovary syndrome, fatty liver disease, cachexia, obesity, arteriosclerosis, stroke, gall stones, inflammatory bowel disease, inherited metabolic disorders such as lipid storage disorders, and the like. In addition, various conditions associated with hyperlipidemia include those described in issued U.S. Pat. No. 8,003,795 (Liu, et al) and 8,044,243 (Sharma, et al), the complete contents of both of which are herein incorporated by reference in entirety.

In some aspects, the diseases and conditions that are prevented or treated include inflammation, and/or diseases and conditions associated with, characterized by or caused by inflammation. These include a large group of disorders which underlie many human diseases. In some embodiments, the inflammation is acute, resulting from e.g. an infection, an injury, etc. In other embodiments, the inflammation is chronic. In some embodiments, the immune system is involved with the inflammatory disorder as seen in both allergic reactions and some myopathies. However, various non-immune diseases with etiological origins in inflammatory processes may also be treated, including cancer, atherosclerosis, and ischemic heart disease, as well as others listed below.

Examples of disorders associated with abnormal inflammation which may be prevented or treated using at least one OCS include but are not limited to: acne vulgaris, asthma, various autoimmune diseases, Celiac disease, chronic prostatitis, glomerulonephritis, various hypersensitivities, inflammatory bowel diseases, pelvic inflammatory disease, reperfusion injury, rheumatoid arthritis, sarcoidosis, transplant rejection, vasculitis, and interstitial cystitis. Also included are inflammation disorders that occur as a result of the use of both legally prescribed and illicit drugs, as well as inflammation triggered by negative cognitions or the consequences thereof, e.g. caused by stress, violence, or deprivation.

In one aspect, the inflammatory disorder that is prevented or treated is an allergic reaction (type 1 hypersensitivity), the result of an inappropriate immune response that triggers inflammation. A common example is hay fever, which is caused by a hypersensitive response by skin mast cells to allergens. Severe inflammatory responses may mature into a systemic response known as anaphylaxis. Other hypersensitivity reactions (type 2 and type 3) are mediated by antibody reactions and induce inflammation by attracting leukocytes which damage surrounding tissue, and may also be treated as described herein.

In other aspects, inflammatory myopathies are prevented or treated. Such myopathies are caused by the immune system inappropriately attacking components of muscle, leading to signs of muscle inflammation. They may occur in conjunction with other immune disorders, such as systemic sclerosis, and include dermatomyositis, polymyositis, and inclusion body myositis.

In one aspect, the methods and compositions of the disclosure, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, are used to prevent or treat systemic inflammation such as that which is associated with obesity, such as inflammation associated with metabolic syndrome and diabetes (e.g. type 2 adult onset diabetes). In such inflammation, the processes involved are identical to tissue inflammation, but systemic inflammation is not confined to a particular tissue but involves the endothelium and other organ systems. Systemic inflammation may be chronic, and is widely observed in obesity, where many elevated markers of inflammation are observed, including: IL-6 (interleukin-6), IL-8 (interleukin-8), IL-18 (interleukin-18), TNF-α (tumor necrosis factor-alpha), CRP (C-reactive protein), insulin, blood glucose, and leptin. Conditions or diseases associated with elevated levels of these markers may be prevented or treated as described herein. In some embodiments, the inflammation may be classified as “low-grade chronic inflammation” in which a two- to threefold increase in the systemic concentrations of cytokines such as TNF-α, IL-6, and CRP is observed. Waist circumference also correlates significantly with systemic inflammatory responses; a predominant factor in this correlation is due to the autoimmune response triggered by adiposity, whereby immune cells “mistake” fatty deposits for infectious agents such as bacteria and fungi. Systemic inflammation may also be triggered by overeating. Meals high in saturated fat, as well as meals high in calories have been associated with increases in inflammatory markers, and the response may become chronic if the overeating is chronic.

Implementation of the methods of the disclosure will generally involve identifying patients suffering from or at risk for developing conditions associated with high cholesterol and/or lipids, and administering the composition of the present disclosure, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, in an acceptable form by an appropriate route. The exact dosage to be administered may vary depending on the age, gender, weight and overall health status of the individual patient, as well as the precise etiology of the disease. However, in general for administration in mammals (e.g. humans), dosages (in terms of the OCS) in the range of from about 0.1 to about 100 mg or more of compound per kg of body weight per 24 hr., and preferably about 0.1 to about 50 mg of compound per kg of body weight per 24 hr., and more preferably about 0.1 to about 10 mg of compound per kg of body weight per 24 hr. are effective.

### Liver Disorders

The liver is responsible for the maintenance of lipid homeostasis in the body, and the compositions described herein may be used prevent and treat liver disease and damage of the liver per se (e.g. NAFLD), and to prevent and treat diseases associated with excessively high levels of circulating lipids, i.e. to prevent or treat hyperlipidemia and associated disorders such as atherosclerosis. In some aspects, the subjects treated by the compositions and methods described herein, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, have at least one symptom of or have been diagnosed with non-alcoholic fatty liver disease (NAFLD) and/or nonalcoholic steatohepatitis (NASH).

In further aspects, the subjects treated by the compositions and methods described herein, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, have at least one symptom of and/or have been diagnosed with a liver disorder such as hepatitis, inflammation of the liver, caused mainly by various viruses but also by some poisons (e.g. alcohol); autoimmunity (autoimmune hepatitis) or hereditary conditions; non-alcoholic fatty liver disease, a spectrum in disease, associated with obesity and characterized by an abundance of fat in the liver, which may lead to hepatitis, i.e. steatohepatitis and/or cirrhosis; cirrhosis, i.e. the formation of fibrous scar tissue in the liver due to replacing dead liver cells (the death of liver cells can be caused, e.g. by viral hepatitis, alcoholism or contact with other liver-toxic chemicals); haemochromatosis, a hereditary disease causing the accumulation of iron in the body, eventually leading to liver damage; cancer of the liver (e.g. primary hepatocellular carcinoma or cholangiocarcinoma and metastatic cancers, usually from other parts of the gastrointestinal tract); Wilson's disease, a hereditary disease which causes the body to retain copper; primary sclerosing cholangitis, an inflammatory disease of the bile duct, likely autoimmune in nature; primary biliary cirrhosis, an autoimmune disease of small bile ducts; Budd-Chiari syndrome (obstruction of the hepatic vein); Gilbert's syndrome, a genetic disorder of bilirubin metabolism, found in about 5% of the population; glycogen storage disease type II; as well as various pediatric liver diseases, e.g. including biliary atresia, alpha-1 antitrypsin deficiency, alagille syndrome, and progressive familial intrahepatic cholestasis, etc. In addition, liver damage from trauma may also be treated, e.g. damage caused by accidents, gunshot wounds, etc. Further, liver damage caused by certain medications may be prevented or treated, for example, drugs such as the antiarrhythmic agent amiodarone, various antiviral drugs (e.g. nucleoside analogues), aspirin (rarely as part of Reye's syndrome in children), corticosteroids, methotrexate, tamoxifen, tetracycline, etc. are known to cause liver damage.

In other aspects, the disclosure involves a method for promoting liver cell proliferation or liver tissue regeneration in a subject, comprising administering a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, to a subject in need of at least one of liver cell proliferation and liver tissue regeneration, in order to promote proliferation of liver cells or regeneration of liver tissue in the subject. In some aspects, administration is performed before, during or after liver surgery in the subject, for example, liver transplant surgery. The subject may also have at least one of cirrhosis, liver injury, and hepatitis.

### Leptin Deficiency, Leptin Resistance and Lipid Storage Disease

The present disclosure also provides compositions and methods for the treatment of disorders characterized by abnormal lipid accumulation (LA). Administration of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, to mammals which have existing abnormal, harmful deposits of lipids (e.g. lipid globules in liver or other organs or tissues wherein deposition is inappropriate), results in a decrease or elimination of the lipid deposits and the prevention of additional lipid accumulation. Thus, administration prevents abnormal lipid deposition and reverses lipid deposition (accumulation) that is extant when treatment begins.

Disorders that are so-treated are referred to herein by phrases such as “lipid accumulation disorders”, “lipid deposition disorders”, etc. and include but are not limited to:

I. disorders which result from a lack or attenuation of leptin activity, due to, for example,
i) a genetic mutation that causes low levels of leptin production, or production of a non- or poorly functioning leptin molecule, such as occurs in leptin deficiency (LD); or
ii) a defect in leptin signaling, caused by e.g. a congenital or acquired abnormality or deficiency in the functioning of the leptin receptor, e.g. due to a genetic mutation of the leptin receptor, or due to an acquired loss of receptor sensitivity to leptin binding such as that which occurs in leptin resistance (LR); and

II. lipid storage disorders, which are generally congenital.

The term “attenuated leptin activity” as used herein thus embraces leptin deficiency (LD) and leptin resistance (LR) as characterized in i) and ii) above. Similarly, the term “leptin-deficiency associated lipid accumulation” as used herein embraces lipid accumulation associated with leptin deficiency (LD) and leptin resistance (LR), as characterized in i) and ii) above.

Thus, subjects treated by the compositions and methods described herein may have at least one symptom of leptin deficiency and/or leptin resistance and/or a lipid storage disease. These subjects may or may not have i) a genetic mutation that causes low levels of leptin production, or production of a non- or poorly functioning leptin molecule, such as occurs in leptin deficiency (LD) (e.g. a mutation in the LEP gene encoding leptin); or ii) a defect in leptin signaling, caused by e.g. a congenital or acquired abnormality or deficiency in the functioning of the leptin receptor, e.g. due to a genetic mutation of the leptin receptor, (e.g. mutations in the Ob (lep) gene that encodes the leptin receptor) or due to an acquired loss of receptor sensitivity to leptin binding such as that which occurs in leptin resistance (LR); or iii), a lipid storage disorder, which may be congenital. Lipid storage disorders include, for example, neutral lipid storage disease, Gaucher disease, Niemann-Pick disease, Fabry disease, Farber's disease, gangliosidoses such as GM1 gangliosidoses and GM2 gangliosidoses (e.g. Tay-Sachs disease and Sandhoff disease), Krabbe disease, metachromatic leukodystrophy (MLD, including late infantile, juvenile, and adult MLD), and acid lipase deficiency disorders such as Wolman's disease and cholesteryl ester storage disease.

The methods involve administering an amount of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, that is a therapeutically effective to prevent or treat the disease or condition.

### Skin Inflammation

In yet further aspects, subjects who are treated with the compositions and methods described herein have been diagnosed with an “inflammatory skin disease” or an “inflammatory skin disorder” and/or are afflicted with one or more skin lesions. Inflammatory skin diseases are typically characterized by, for example, reddened, itchy, dry, rough, flaky, inflamed, and irritated skin, and the skin may also exhibit blisters, scaly plaques, etc. In some aspects, the inflammatory skin disease is acute, generally resolving within days or weeks even if untreated, and the compositions and methods of the disclosure ameliorate symptoms during disease resolution (e.g. lessen itching, redness, etc.) and/or hasten the disappearance of symptoms. Alternatively, in some aspects, the skin inflammatory disease/disorder is chronic, e.g. without treatment, or even with conventional treatment, symptoms persist for weeks, months, or years, or even indefinitely. In some aspects, the compositions and methods of the disclosure ameliorate (provide relief from) symptoms of chronic skin inflammation while the disease persists (e.g. lessening itching, redness, cracking and flaking of skin, etc.) and/or also partially or completely cure (cause the complete or nearly complete disappearance of) symptoms which would otherwise be present.

“Inflammatory skin diseases” is intended to encompass diseases and conditions caused by exposure to specific, known or identifiable etiological agents, and also diseases/conditions whose causes are less well-defined, e.g. they are due to an immune disorder or malfunction (e.g. an autoimmune reaction), to stress, to an unidentified allergy, to a genetic predisposition, etc., and/or are due to more than one factor.

A “skin lesion” as used herein refers most generally to an area of the skin that has abnormal growth or appearance compared to the skin around it. For example, the area of the skin may be one exhibiting a breach of one or more of the outer skin layers (at least the epidermis, and possibly the dermis and/or subcutis (hypodermis) which exposes underlying tissue. Skin lesions include, for example, skin ulcers i.e. a local defect, breakdown or excavation of the surface of the skin produced by sloughing of necrotic inflammatory tissue. Ulcers may be, for example, neurotrophic or ischemic in nature, including decubitous ulcers, diabetic ulcers, (which are frequently foot ulcers), etc. The treatment of venous and arterial ulcers, typically of the leg or foot, is also encompassed. Skin lesions also include those caused by deliberate or accidental breaches, e.g. cuts, scratches, incisions, etc., with or without accompanying inflammation or infection. A skin lesion may also be referred to as a sore, open sore, etc. The underlying cause of a skin lesion may be inflammation, infection (e.g. viral or bacterial infection), neuropathy, ischemia, necrosis (e.g. as occurs in diabetic ulcers), or a combination of one or more of these. In addition, many skin diseases are caused by and/or characterized by both inflammation and one or more skin lesions, and all such skin diseases and/or lesions, or symptoms thereof, can be treated by the compositions and methods disclosed herein.

For the avoidance of doubt, skin lesion includes skin necrosis. Thus, the methods and techniques described herein are suitable for treating or prophylactically treating skin necrosis.

Inflammatory skin diseases/disorders (particularly chronic inflammatory skin diseases), include but are not limited to, for example: atopic dermatitis, all types of psoriasis, acne, ichthyosis, contact dermatitis, eczema, photodermatoses, dry skin disorders, herpes simplex, zoster (shingles), sunburn (e.g., severe sunburn), etc. References herein to psoriasis refer to all types of psoriasis unless otherwise specified.

In some aspects, the disease/condition that is treated is psoriasis, including all types of psoriasis such as plaque flexural, guttate, pustular, nail, photosensitive, and erythrodermic psoriasis. Psoriasis is generally recognized as an immune disorder and may be triggered by or associated with factors such as infection (e.g. strep throat or thrush), stress, injury to skin (cuts, scrapes, bug bites, severe sunburns), certain medications (including lithium, antimalarials, quinidine, indomethacin), etc. and may be comorbid with other immune conditions such as Crohn's disease, type 2 diabetes, cardiovascular disease, high blood pressure, high cholesterol, depression, ulcerative colitis, etc. Psoriasis due to any of these causes, or any other cause or an unknown cause, may be treated by the formulations and methods described herein.

In some aspects, the disease/condition that is treated is eczema. Eczema is a general term used to describe a variety of conditions that cause an itchy, inflamed skin rash, and refers to any superficial inflammatory process involving primarily the epidermis, marked early by redness, itching, minute papules and vesicles, weeping, oozing, and crusting, and later by scaling, lichenification, and often pigmentation. Various types of eczema are known, including asteatotic eczema, eczema herpeticum, nummular eczema, neurodermatitis, xerotic eczema erythema (dry scaling, fine cracking, and pruritus of the skin, occurring chiefly during the winter when low humidity in heated rooms causes excessive water loss from the stratum corneum), and atopic dermatitis.

Atopic dermatitis, a form of eczema, is a non-contagious disorder characterized by chronically inflamed skin and sometimes intolerable itching. Atopic dermatitis refers to a wide range of diseases that are often associated with stress and allergic disorders that involve the respiratory system, like asthma and hay fever. Although atopic dermatitis can appear at any age, it is most common in children and young adults, e.g. infantile eczema. Characterized by skin that oozes and becomes encrusted, infantile eczema most often occurs on the face and scalp. In one aspect, the atopic dermatitis is contact allergic dermatitis, caused, for example, by exposure to an agent that causes an allergic reaction. Common triggers of atopic dermatitis include, for example, soap and household cleaners (e.g. all-purpose cleaners, dish detergents, laundry detergent, window cleaners, furniture polish, drain cleaners, toilet disinfectants, etc.); clothing (e.g. rough fabrics like wool); heat; contact with latex; cosmetics and ingredients of cosmetics (e.g. ascorbic acid, paraban preservatives, and alpha hydroxy acids such as glycolic acid, malic acid, and lactic acid); oils from plants such as poison ivy, poison oak, and poison sumac; contact with foods, especially acidic foods or spices; nickel, a common component of costume jewelry, watchbands, zippers, etc.; sunscreen and ingredients thereof, e.g. para-aminobenzoic acid (PABA)-based chemicals; etc.

Methods of the present description include administering an amount of a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, that is a therapeutically effective to prevent or treat the disease or condition.

### Prevention/Treatment of Two or More Diseases/Conditions

In some aspects, the subjects treated by the compositions and methods described herein receive treatment with two or more separate compositions, each of which comprises at least one OCS, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, and each of which is prescribed or used for a different disease or condition. For example, a subject that is taking an oral dosage form of an OCS (e.g. as described in U.S. Pat. No. 8,399,441), or a composition as described herein, to treat high cholesterol, may also be treated for a different disorder e.g. acute liver failure due to APAP overdose, with an IV formulation of a different composition as described herein, or even with a third composition such as a topical formulation to treat e.g. contact dermatitis. The different compositions may have different properties, e.g. the form may differ (e.g. a tablet vs liquid vs cream), the mode or delivery may differ (e.g. oral vs intravenous vs topical) and the concentration of OCS and other components in the composition may differ to suit the particular disease or condition. The recommended dosing regimen and the duration of the treatment may also differ but may overlap, e.g. a patient may be treated for dermatitis with a topical cream while taking an oral preparation (e.g. a capsule) for high cholesterol and/or while being treated for ALF due to an APAP overdose. The treatment for high cholesterol may involve a regimen of one daily tablet for many years with a relatively low dosage of OCS; the treatment for dermatitis may involve application of a cream twice daily until symptoms disappear; and the treatment for acute liver failure due to APAP overdose may involve administration of large volumes of a composition as described herein with very high OCS concentrations, and lower amounts (e.g. 5% or less), in one or two boluses.

### Description of Administration of the Compositions

Implementation of the methods generally involves identifying patients suffering from or at risk of developing a disease or condition described herein, and administering a composition as described herein, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, by an appropriate route. The exact dosage to be administered may vary depending on the age, gender, weight and overall health status of the individual patient, or on other treatments being received by the patient, as well as the extent or progression of the disease condition being treated and the precise etiology of the disease. However, in general for administration in mammals (e.g. humans), sufficient composition is administered to achieve OCS dosages in the range of from about 0.001 to about 100 mg or more per kg of body weight per 24 hr., and preferably about 0.01 to about 50 mg of compound per kg of body weight per 24 hr., and more preferably about 0.1 to about 10 mg of compound per kg of body weight per 24 hr. are effective. Daily doses (in terms of OCS) generally range from about 0.1 milligram to about 5000 milligrams per person per day. In some aspects, the dose is from about 10 milligrams to about 2000 milligrams per person per day, or about 100 milligrams to about 1000 milligrams per person per day. The dose will vary with the route of administration, the bioavailability, and the particular formulation that is administered, as well as according to the nature of the malady that is being prevented or treated.

Administration may be oral or parenteral, including intravenously, intramuscularly, subcutaneously, intradermal injection, intraperitoneal injection, etc., or by other routes (e.g. transdermal, sublingual, rectal and buccal delivery, inhalation of an aerosol, intravaginally, intranasally, topically, as eye drops, via sprays, by iontophoresis, by photoacoustic-guided drug delivery, microneedle delivery, etc. The route of administration typically depends on the nature of the condition that is treated and on e.g. whether the treatment is prophylactic or intended to effect a cure of disease that is present. For example, to achieve a preventative effect before organ dysfunction has occurred, oral dosing may be sufficient, especially in view of the excellent bioavailability of orally administered OCS. Further, administration of the compound by any means may be carried out as a single mode of therapy, or in conjunction with other therapies and treatment modalities, e.g. surgery, other medicaments (e.g. pain medications, etc.), neutraceuticals, diet regimens, exercise, etc. In some aspects, the product involves a ready to use product solution that can be administered by intravenous bolus, intravenous infusion (upon dilution with pharmaceutically appropriate diluents), intramuscular, subcutaneous, or oral routes.

The subject to whom the composition is administered is generally a mammal, frequently a human, but this is not always the case. Veterinary applications of this technology are also contemplated, e.g. for companion pets (cats, dogs, etc.), or for livestock and farm animals, for horses, and even for “wild” animals that have special value or that are under the care of a veterinarian, e.g. animals in preserves or zoos, injured animals that are being rehabilitated, etc.

In some aspects, the compositions are administered in conjunction with other treatment modalities such as various pain relief medications, anti-arthritis agents, various chemotherapeutic agents, antibiotic agents, various intravenous fluids (e.g. saline, glucose, etc.), and the like, depending on the malady that is afflicting the subject. “In conjunction with” refers to both administration of a separate preparation of the one or more additional agents, and also to inclusion of the one or more additional agents in a composition of the present disclosure. For example, aspirin, ibuprofen and acetaminophen, which all have potential serious organ-damaging side effects when taken long term, or when taken by certain vulnerable groups (e.g. the very young, the elderly, etc.), or when overdoses are ingested, etc., may be administered by inclusion in a composition as described herein. Accordingly, dosage forms comprising at least one OCS and at least one of polyalkylene glycol, carboxymethyl cellulose or pharmaceutically acceptable salt thereof, and polyoxylglyceride, and one or more of such agents are contemplated.

The administration of the compound (i.e., composition) of the present disclosure, e.g., a composition including at least one OCS and at least one of polyalkylene glycol, carboxymethyl cellulose or pharmaceutically acceptable salt thereof, and polyoxylglyceride as described herein, e.g., as described in the separately numbered aspects described herein, may be intermittent, or at a gradual or continuous, constant or controlled rate. In addition, the time of day and the number of times per day that the pharmaceutical formulation is administered may vary and are best determined by a skilled practitioner such as a physician. For example, for treatment of an APAP overdose, the compound may be administered within 1 week, such as within 1 day, within 12 hours, within 4 hours, within 1 hour, or within 10 minutes, of an overdose e.g. of an agent that causes organ damage. The compound may be administered at least once a day (e.g., twice daily) before surgery for at least 1 month or at least 1 week, or at least 1 day before surgery, or even during surgery, e.g. surgery related to or associated with or which may cause organ failure (e.g. surgery that involves intentional ischemia/reperfusion). The compound may also be administered on at least a daily basis (e.g., twice daily) after surgery for at least 1 day, at least 1 week, or at least 1 month. For example, the surgery may be heart surgery (e.g., coronary artery bypass grafting (CABG)), cardiovascular surgery, heart-lung transplant, lung surgery (e.g., pulmonary embolism surgery), deep vein thrombosis (DVT) surgery, brain surgery, liver surgery, bile duct surgery, kidney surgery (e.g., kidney stone surgery), gastrointestinal surgery (e.g., intestinal, intestinal blockage, diverticulitis, or intestinal torsion surgery), or aneurysm surgery. In some cases, such as when one or more organs to be treated comprises a liver, the administering may occur for not more than 14 days, such as not more than 10 days, not more than 8 days, not more than 5 days, or not more than 1 day.

The compositions (preparations) of the present disclosure, e.g., compositions including at least one OCS and at least one of polyalkylene glycol, carboxymethyl cellulose or pharmaceutically acceptable salt thereof, and polyoxylglyceride as described herein, e.g., as described in the separately numbered aspects described herein, may be formulated for administration by any of the many suitable means which are known to those of skill in the art, including but not limited to: orally, by injection, rectally, by inhalation, intravaginally, intranasally, topically, as eye drops, via sprays, etc. In some aspects, the mode of administration is oral, by injection or intravenously. Typically, oral administration is particularly effective when used prophylactically, e.g. to prevent organ damage (e.g. caused by or necrosis and/or apoptosis) and that would otherwise occur in a patient who is taking an organ-damaging agent and/or is exposed to a toxic agent such as radiation, either acutely or for a prolonged period of time, e.g. weeks, months or years. When damage has already occurred, and especially when disease symptoms are already evident, the route of administration is generally parenteral or intravenous to speed delivery of the active agents in the composition.

In some cases, a method of administering comprises injecting a suspension comprising particles comprising one or more oxygenated cholesterol sulfate (OCS) suspended in a vehicle comprising a hydrophilic polymer.

In some cases, a method of making a suspension comprises mixing particles comprising one or more oxygenated cholesterol sulfate (OCS) with a vehicle comprising at least one polyalkylene glycol to form a suspension. In other cases, a method of making a suspension comprises mixing particles comprising one or more oxygenated cholesterol sulfate (OCS) with a vehicle comprising at least one carboxymethyl cellulose or pharmaceutically acceptable salt thereof to form a suspension. In other cases, a method of making a suspension comprises mixing particles comprising one or more oxygenated cholesterol sulfate (OCS) with a vehicle comprising at least one polyoxylglyceride to form a suspension.

In some aspects, the mixing comprises manual shaking. In some aspects, the mixing comprises sonication. In other aspects, the mixing comprises shaking in a flat bed shaker.

In some aspects, the method of making comprises homogenizing the suspension.

In some cases, the method of making comprises jet milling one or more oxygenated cholesterol sulfate to form the particles.

In some aspects, the method of making comprises sieving one or more oxygenated cholesterol sulfate to select the particles for the mixing.

In some aspects, the method of making comprises sterilizing the particles prior to the mixing. In some cases, the method of making comprises autoclaving the particles prior to the mixing. In some cases, the method of making comprises gamma irradiating the particles prior to the mixing.

The present disclosure will be further illustrated by way of the following Examples. These Examples are non-limiting and do not restrict the scope of the disclosure. Unless stated otherwise, all percentages, parts, etc. presented in the Examples are by weight.

### EXAMPLES

### Example 1 (Particle Preparation)

### BACKGROUND

Two lots of 25HC3S sodium salt (Lot #A and Lot #B) were first passed through either 20 mesh or 35 mesh stainless steel sieves for particle size analysis. The particle sizes were further reduced by jet milling and analyzed again. All particle size analyses were determined using a Malvern Mastersizer 2000.

### Equipment

Fluid Energy Model 00 Jet-O-Mizer was used for all jet-milling. Malvern Mastersizer 2000 equipped with a Hydro 2000S dispersion cell was used for particle size analysis.

### Methods

(a) Particle Size Reduction Conditions for 25HC3S

| Lot # of | Batch size | | Feed rate |
|---|---|---|---|
| 25HC3S | (g) | Particle size reduction method | (g/min) |
| A | 0.2569 | Pressed through 35 mesh screen | NA |
| | | manually with a stainless steel | |
| | | spatula | |
| | 3.0504 | Pressed through 20 mesh screen | NA |
| | | manually with a stainless steel | |
| | | spatula | |
| | 5.999 | Passed through 20 mesh screen, | Not |
| | | followed by Jet milling-1^{st} pass | controlled |
| | | Passed through 20 mesh screen, | |
| | | followed by Jet milling-2^{nd} pass | |
| | | Passed through 20 mesh screen, | |
| | | followed by Jet milling-3^{rd} pass | |
| B | NA | Passed through 20 mesh screen | NA |
| | 4.396 | Passed through 20 mesh screen, | Not |
| | | Jet milling, 1^{st} pass (1^{st} sample) | controlled |
| | | Passed through 20 mesh screen, | |
| | | Jet milling, 2^{nd} pass | |
| | | Passed through 20 mesh screen, | |
| | | Jet milling, 3^{rd} pass | |
| | 6.000 | Passed through 20 mesh screen, | 1 |
| | | Jet milling, 1^{st} pass (2^{nd} sample) | |

(b) Sample Preparation for Particle Size Analysis:

Approximately 60 mg of API was weighed into a 4 mL screw cap vial and 1 mL water, USP was added to the vial. The sample was manually shaken 15 times twice to form a homogeneous suspension. Approximately 0.21 to 0.35 mL of suspension or paste (Lot #B formed paste after one sample analysis) was added to the dispersion cell for analysis with the resulting obscuration in the range of 5-15%. Duplicate samples from each single sample preparation were analyzed.

(c) Particle Size Analysis Parameters:

Particle refractive index was assumed to be 1.53 (not measured by refractometer) and particle absorption index was 0.01.
Dispersant (water, USP, presaturated with 25HC3 S) refractive index was 1.33. Pump condition: After adding the suspension to Hydro 2000S dispersion cell, the sample was pumped at 3000 rpm and sonicated at 100% for 2 min, followed by pumping only for 3 min prior to particle size measurement. Throughout the measurement, the pump rate was 3000 rpm without sonication.
The measurement integration time was 20,000 ms; the numbers of measurements for each sample were 5 with a 20 seconds delay in between two measurements.
Analysis model: General purpose

### Results and Discussion

The particle sizes for 25HC3S (Lot #A and Lot #B) are summarized in Table A. As shown in Table A, there is no significant difference in d(0.9), size of particle for which 90% of sample is below this size, for 25HC3 S Lot #A between jet milling—1^{st} pass (5.180 μm) and jet milling—3^{rd} pass (2.755 μm). There is also no significant difference in d (0.9) between jet milling—1^{st} pass (22.07 μm) and jet milling—3^{rd} pass (16.17 μm) for 25HC3S Lot #B. D (0.9) is 9.09 μm with feed rate of 1 g/min, compared to that of 16.17 μm with uncontrolled feed rate for Lot #B, jet-milled—1^{st} pass.

TABLE A
Summary Table for Particle Size Analysis^{1, 2} of 25HC3S (Lot# A and Lot# B)
by Malvern Mastersizer 2000 Equipped with a Hydro 2000S Dispersion Cell

| | Lot# A | | | Lot# B | | |
|---|---|---|---|---|---|---|
| Particle size reduction | d (0.1) ³ | d (0.5) ³ | d ( 0.9) ³ | d (0.1) ³ | d (0.5) ³ | d ( 0.9) ³ |
| prior to analysis | (μm) | | | (μm) | | |
| Press through 35 mesh | 2.536 | 7.147 | 22.195 | No 35 mesh | | |
| sieve with a stainless | | | | material available | | |
| steel spatula | | | | | | |
| Press through 20 mesh | 2.268 | 6.324 | 18.712 | 4.132 | 15.439 | 43.740 |
| sieve with a stainless | | | | | | |
| steel spatula | 2.474 | 6.905 | 20.290 | 4.947 | 21.437 | 68.250 |
| Press through 20 mesh | | | | 2.148 | 6.858 | 29.028 |
| sieve with a stainless | 0.705 | 2.091 | 5.180 | 1.822 | 5.126 | 22.070 |
| steel spatula then Jet | | | | | | (1^{st} milling |
| milling 1^{st} pass | | | | | | sample) |
| Press through 20 mesh | 0.384 | 1.214 | 4.026 | 1.431 | 4.192 | 15.878 |
| sieve with a stainless | | | | | | |
| steel spatula then Jet | 0.496 | 1.320 | 4.035 | 1.109 | 3.920 | 13.196⁴ |
| milling 2^{nd} pass | | | | | | |
| Press through 20 mesh | 0.137 | 0.664 | 2.896 | 0.976 | 4.251 | 16.170 |
| sieve with a stainless | | | | | | 47.759⁴ |
| steel spatula then Jet | 0.120 | 0.580 | 2.755 | 0.651 | 2.329 | (air bubble |
| milling 3^{rd} pass | | | | | | possibly |
| | | | | | | caused |
| | | | | | | high value) |
| Press through 20 mesh | ND | | | 1.253 | 3.172 | 9.090 |
| sieve with a stainless | | | | | | (2^{nd} milling |
| steel spatula then Jet | | | | | | sample) |
| milling 1^{st} pass | | | | | | |

¹Sample prep: H2O (1 mL) was added to a 4 mL vial containing ~60 mg of 25HC35. The suspension was manually shaken 15 times twice prior to analysis
² Sample size: 0.21-0.35 mL of 25HC35 with sample concentration ~60 mg/mL in H2O
Dispersant is water with refactive index (RI) = 1.33. Particle is 25HC35 with refractive index (RI) = 1.53, Particle absorption index is set at 0.01.
Sample analysis: Pump at 3000 rpm with sonication at 100% for 2 min, then pumping at 3000 rpm without sonication for 2 min prior to measurement. During the measurement, only pumping at 3000 rpm without sonication was used. Analysis model: general purpose.
³ Average of 5 consecutive measurements with 20 seconds for each measurement.
⁴Sample formed thick paste.

### Example 2A (Suspension Preparation)

### INTRODUCTION

This Example includes a total of 19 studies in the development of 25HC3S sodium salt suspension formulations. 25HC3S shows low solubility in various aqueous solutions and FDA approved organic solvents or oils. Therefore, suspension formulations were chosen as dosage forms for 25HC3S, e.g., for subcutaneous injection.

Two lots of 25HC3S sodium salt (Lot #A and Lot #B) were used. Lot #B was delumped through a 20 mesh screen. The drug substance was either used directly or further jet-milled, prior to the preparation of suspensions for the studies. A third lot of 25HC3 S sodium salt (Lot #C) was jet-milled first and then used directly or further passed through a 20 mesh screen prior to the studies. More than 10 vehicles were screened. Four mixing methods were evaluated: manual shaking (Mixing Method 1), manual shaking followed by sonication (Mixing Method 2) and homogenization with a sonic probe (Mixing Method 3) as well as mechanical shaking horizontally in a flat bed shaker (Mixing Method 4). Studies #1 to 13 combined vehicle screening, mixing methods and syringeability evaluation. The effect of drug concentrations on the syringeability was evaluated (Studies #10 and 11).

25HC3S in 3% PEG 3350 plus 0.3% Tween 80 and 0.7% NaCl with 0.15% L-Methionine in 10 mM phosphate buffer at pH 7.4 (Vehicle PEG 3350 with L-Methionine) was initially chosen as a preferred suspension formulation based on Studies #1-11. After storage at room temperature for a few months, the preferred suspension formulation produced a sulfur-like odor which might be due to the degradation of L-Methionine. L-Methionine was initially added as an antioxidant. A stress study for 25HC3 S suspension formulation with hydrogen peroxide showed that oxidative degradation did not occur for 25HC3S. Therefore, L-Methionine was removed from the preferred suspension formulation. 25HC3S in Vehicle PEG 3350 (without L-Methionine) was used for further syringeability study (Studies #12 and 13).

Studies #14-16 evaluated homogeneity, and Study #17 evaluated stability for the 25HC3 S preferred suspension formulation (with L-Methionine) at 10 to 25 mg/mL by HPLC analysis. The HPLC technique involved reverse phase HPLC for measuring the concentration of 25HC3 S in the solubility samples.

25HC3 S preferred suspension formulation (without L-Methionine) at 25 mg/mL was further improved to meet the isotonic condition (osmolality of approximately 300 mmole/kg) by increasing NaCl from 0.7% to 0.75% (Study #18).

The final composition of the improved suspension formulation was 25HC3S at 25 mg/mL in 3% PEG 3350 plus 0.3% Tween 80 and 0.75% NaCl in 10 mM phosphate aqueous buffer at pH 7.4. The suspension was prepared by Mixing Method 4 (shaken in a flat-bed shaker at 200 rpm for 45 minutes) with Jet-milled drug. The osmolality for the final improved formulation was 321 mmole/kg (Study #18). The homogeneity ranged from 89.3-105.9% label strength (Study #19). This suspension formulation was used for the rat Imquimod-induced psoriasis-like inflammation study on mice of below-noted Example 3.

### Experimental

### (A) Materials:

Active Pharmaceutical Ingredient (25HC3S): Lot #A was delumped through a 20 mesh screen using a stainless steel spatula, with or without subsequent jet milling. Lot #B was delumped through a 20 mesh screen and jet milled. Lot #C was jet-milled, with or without subsequently being passed through a 20 mesh screen.

Inactive ingredients:

Inactive Ingredients for the Suspension Vehicles

| | | Vendor or |
|---|---|---|
| Name/Grade | Function | Manufacturer |
| PEG 3350/NF | Solubility enhancer | Spectrum |
| | or wetting agent | |
| Plasdone C17 | Solubility enhancer | Ashland |
| | or wetting agent | |
| Tween 80 | Surfactant | Spectrum |
| (Polysorbate 80)/NF | | |
| L-Methionine/USP | Antioxidant | Sigma Aldrich |
| | | JT baker |
| Mannitol/USP | Osmolality | Spectrum |
| | adjustment | VG0692 |
| Sodium carboxymethyl | Viscosity enhancer | Spectrum |
| cellulos (NaCMC) | Thickening agent | |
| Sodium phosphate, | Buffering agent for | Spectrum |
| monobasic | pH adjustment | |
| monohydrate/USP | | |
| Sodium phosphate, | Buffering agent for | JT Baker |
| dibasic, | pH adjustment | |
| anhydrous/USP | | |
| Sodium chloride | Osmolality | BDH |
| (NaCl)/ACS | adjustment | |
| Water (H₂O)/USP | Solvent | Durect |
| Sesame oil, NF | Solvent | Spectrum |
| Propylene Glycol | Solvent | Sigma Aldrich |
| (PG)/USP | | |
| Benzyl benzoate | Solvent | Spectrum |
| (BB)/USP | | |
| Benzyl alcohol | Solvent | Spectrum |
| (BA)/NF | | |

### (B) Equipment and Supplies

Equipment:

Jet Mill: Fluid Energy Model 00 Jet Mill

Sonicator: Branson, Model 8510

Homogenizer: PowerGen 1000 attached to a 5×95 mm flat probe

Flat bed shaker: IKA Digital shaker, Model HS501

Vapor Pressure Osmometer: Vapor® Vapor Pressure Osmometer, Model

5520 (Wescor, Inc.)

HPLC System: Agilent 1100 HPLC System

Supplies

Syringe: 1 mL BD syringe (luer lok tip), reference no: 309628 Needles for syringeability study: listed below

| Vendor | Gauge and length of needle |
|---|---|
| Terumo | UTW*, 20G1” |
| | UTW, 21G1” |
| | UTW, 22G1” |
| | UTW, 25G5/8” |
| BD | Tuberculin syringe attached to |
| | 27G1/2” needle |
| | 21G1” |
| | 22G1” |
| | 20G1.5” |
| | 21G1.5” |
| Excel | 23G1” |

*UTW = ultra thin wall

### (C) Suspension Formulations Preparation

Preliminary Suspension Formulations Preparation for Syringeability Studies (Studies #1-11)

Weigh approximately 10 to 100 mg each of 25HC3S into 2 mL vials. Add to each vial, 1 mL of vehicle. A total of 3 mixing methods were used to prepare the suspensions. Mixing Method 1: Each vial was manually shaken for 15 to 45 times. The suspension was inspected visually for sedimentation after stored at room temperature (RT) for one minute. The suspension was re-shaken 15 times manually without sonication for syringeability study. Mixing Method 2: Each vial was manually shaken 30 times followed by sonication for 3 or 6 minutes for syringeability study. Mixing Method 3: Each vial was homogenized with a PowerGen1000 homogenizer attached to a 5×95 mm flat probe at speed setting of 4 for 30 or 60 seconds for syringeability Studies #7, 10 and 11. A total of 11 studies were conducted.

Preferred Suspension Formulation Preparation (25HC3 S at 25 mg/mL in Vehicle PEG 3350 without L-Methionine) for Syringeability Studies (Studies #12-13)

Weigh approximately 125 mg (Study #12) or 75 mg (Study #13) each of 25HC3S (Lot #C, jet milled with or without passing through 20 mesh screen) into 10 mL vials. Add to each vial, 5 mL or 3 mL of vehicle to a final 25HC3S concentration of 25 mg/mL. The vial was placed horizontally in a flat bed shaker, shaken at 100 rpm (Study #12) and 200 rpm (Study #13) for up to 45 minutes (Mixing Method 4).

Preferred Suspension Formulation Preparation (in Vehicle PEG 3350 with L-Methionine) for the Homogeneity Study (Studies #14 and 15) and Stability Study (Study #17)

Weigh 80 mg of 25HC3S (Lot #B, Passed through 20 mesh screen and Jet-milled, 3rd pass) into a 10 mL vial. Add to the vial, 8 mL of Vehicle PEG 3350 (with 0.15% L-Methionine and 0.9% NaCl). The suspension was mixed by being manually shaken 30 times followed by sonication for 30 minutes with a Branson Model 8510 sonicator (Mixing Method 2). The suspension at 10 mg/mL was inverted 10 times manually prior to dispensing 1 mL each into 10 mL volumetric flasks for dilution with MeOH for HPLC. A total of 9 samples were dispensed using 1 mL BD syringes attached to 20G1″ or 25G⅝″ Terumo UTW needles for homogeneity analysis by HPLC (Study #14) and stability study (Study #17).

Weigh 50 and 80 mg of 25HC3S into 2 and 10 mL vials, respectively. Add to the vial, 2 mL and 8 mL of Vehicle PEG 3350 with L-Methionine. The suspension was mixed by being manually shaken 30 times followed by sonication for 30 minutes with a Branson Model 8510 sonicator (Mixing Method 2). The vial was inverted 10 times prior to dispensing 0.2 or 0.9 mL into volumetric flasks for methanol dilution for HPLC analysis (total 8 and 7 samples, respectively for HPLC analysis for potency and stability (Study #15).

Preferred Suspension Formulation Preparation (in Vehicle PEG 3350 without L-Methionine) for the Homogeneity Study (Study #16)

Weighed approximately 125 mg each of 25HC3S (Lot #C, jet milled and passed through a 20 mesh screen) into a 10 mL vial. Added to vial, 5 mL of Vehicle PEG 3350 without L-Methionine. The vial was placed horizontally in a flat bed shaker, shaken at 100 rpm for up 45 minutes (Mixing Method 4). There were some small wet lumps stuck to the wall and bottom of the glass vial. The suspension formulation was withdrawn using a lml BD syringe attached to a 25G⅝″ Teruma UTW needle to withdraw and dispense 100 μL or 300 μL each in duplicate at various time points into HPLC vial and diluted to 1/5 with MeOH for the homogeneity analysis by HPLC.

Preferred Formulation Improvement for Isotonicity (Study #18-1)

Vehicle PEG 3350 (3% PEG 3350 plus 0.3% Tween 80 in 10 mM Phosphate at pH 7.4) with 0.71%, 0.77% and 0.80% NaCl were prepared and the osmolality was measured with a vapor pressure osmometer.

Final Improved Suspension Formulation in Vehicle PEG 3350 (3% PEG 3350 plus 0.3% Tween 80 and 0.75% NaCl in 10 mM Phosphate Buffer at pH 7.4) for the Osmolality and Homogeneity Study (Studies #18-2, 19)

Weigh 87 mg of 25HC3S (Lot #C, Jet milled and then pass 20 mesh screen) into a 5-mL vial. Add to the vial 3 mL Vehicle PEG 3350 (without L-methionine and with 0.75% NaCl). The suspension was mixed by shaken in a flat bed shaker at 200 rpm for 45 minutes (Mixing Method 4). The osmolality of 25HC3S suspension at 25 mg/mL was measured (Study #18-2). Weigh another approximately and accurately 90 mg each of 25HC3S (Lot #D and Lot #B, micronized, one pass) into 3 separate 10 mL vials with 3 mL of Vehicle PEG 3350. The vials were placed in a flat bed shaker at 200 rpm for 45 minutes (Mixing Method 4). A 0.4 ml each of suspension was transferred into 2 mL volumetric flasks, using a 1-mL positive displacement pipet and diluted to volume with MeOH for HPLC analysis (a total of 3 vials, each with duplicate analysis). A second set of samples was prepared likewise from the same 3 vials except using 1 mL BD syringe attached to a 27G½″ needle. The homogeneity was determined (Study #19).

### Results and Discussion

### (A) Syringeability Study

A total of 13 studies were conducted for the ease of dispersion and syringeability in various vehicles. The effect of 25HC3S with or without jet milling and the effects of mixing methods as well as drug concentrations on the syringeability were evaluated. The test results were summarized in the following Tables (Tables 1-13) for Study #1 to #13.

### Study #1 (Table 1)

This was a preliminary screening of aqueous and non aqueous suspension vehicles (total of 8 vehicles), using 25HC3 S (Lot #A), delumped through 20 mesh screen with or without being jet milled. All suspensions were mixed by manual shaking (Mixing Method 1) at 30 mg/mL. It was found that 25HC3S was dispersed well in 3% PEG 3350 containing 0.05% Tween 80 in H₂O with good syringeability using a 20G1″ Terumo UTW needle attached to a 1-mL BD syringe. However, some lumps stuck to the needle tip when using a 21G1″ BD needle. 25HC3S was not dispersed as well in 0.5% or 0.25% NaCMC containing 0.05% Tween 80 in H₂O or in sesame oil. The ease of dispersion and syringeability among the vehicles conducted were in the following order: 3% PEG 3350 containing 0.05% Tween 80 in H₂O >0.25-0.5% NaCMC containing 0.05% Tween 80 in H₂O>0.9% NaCl in H₂O=PG/H₂O=50/50>sesame oil=sesame oil containing 0.05% Tween 80=BA/BB (10/90).

25HC3S (Lot #A) was passed through a 20 mesh screen and further jet milled (3^{rd} pass). Some big agglomerates were observed along with fine particles. The big agglomerates and fine particles were also suspended in 3% PEG 3350 containing 0.05% Tween 80 in H₂O separately. It was found the big agglomerate was not dispersed as well (one lump observed). Fine particles (after jet-milled, 3^{rd} pass) dispersed well with no lump observed and good syringeability with 22G1″ Terumo, UTW needle.

This study concluded that 3% PEG 3350 containing 0.05% Tween 80 in H₂O is a better suspension vehicle. 25HC3S did not disperse well in 0.25 or 0.5% NaCMC or sesame oil with or without Tween 80. Jet-milled 25HC3 S showed better syringeability (22G1′ Terumo UTW) in 3% PEG 3350+0.05% Tween 80 in H₂O than that of non-jet-milled 25HC3S (20G1″ Terumo UTW).

### Study #2 (Table 2)

The study evaluated the effect of Tween 80 or 0.9% NaCl on vehicles containing 3% PEG 3350 or 0.5% Plasdone C17 in H₂O (total of 5 vehicles), using 25HC3S (Lot #A) delumped through 20 mesh screen but not jet milled. The concentration for 25HC3 S is 30 mg/mL. After being manually shaken 30 times, no lumps were observed for 25HC3S in 3% PEG 3350 containing 0.05% Tween 80 in H₂O. No sedimentation was observed after 1 minute at room temperature (RT). The suspension was further sonicated for 6 minutes (Mixing Method 2). It showed good syrigeability, using a 25G⅝″ BD needle attached to a 1 mL BD syringe. The ease of dispersion and syringeability for 25HC3 S among the vehicles were in the following order: 3% PEG 3350 containing 0.05% Tween 80 in H₂O>3% PEG 3350+0.05% Tween 80+0.9% NaCl in H₂O=3% PEG 3350+0.9% NaCl in H₂O>0.9% NaCl in H₂O=0.5% Plasdone C17+0.9% NaCl in H₂O.

This study concluded that 3% PEG 3350 was a better solubility enhancer (or wetting agent), compared to 0.5% Plasdone C17. The addition of 0.9% NaCl seemed to decrease the ease of suspension. However, after 3 days at room temperature (RT), the suspension in 3% PEG 3350+0.05% Tween and 0.9% NaCl in H₂O showed no significant sedimentation and re-suspended well. Sonication for 6 minutes improved the syringeability.

### Study #3 (Table 3)

This study evaluated the concentration effect of 25HC3S at 100 mg/mL, using Lot #A delumped through a 20 mesh screen but not jet-milled. The same lot of 25HC3S at 100 mg/mL was suspended in the same vehicles as those in study #2 with 25HC3S at 30 mg/mL. It was found at 100 mg/mL, 25HC3S was not completely suspended in all vehicles with some particles stuck to the wall and the bottom of the vials after being manually shaken 30 times (Mixing Method 1). After 6-minute sonication (Mixing Method 2), it was still somewhat difficult to withdraw the suspension with 20G1″ needle for all vehicles.

The study concluded that the concentration was too high at 100 mg/mL with or without sonication for 25HC3 S (Lot #A, passed through 20 mesh screen but not jet-milled) to completely disperse in all vehicles studied.

### Study #4 (Table 4)

This study evaluated the syringeability of 25HC3S suspensions at 30 mg/mL, using Lot #A passed through 20 mesh screen followed by jet milling (3′ pass). The suspensions showed good syringeability without sonication (Mixing Method 1), using a 20G1″ Terumo needle and with 3 minutes sonication (Mixing Method 2), using a 22G1″ Terumo UTW needles in the vehicles as follows with no lumps observed:

3% PEG 3350+0.3% Tween 80 in H₂O;

3% PEG 3350+0.3% Tween 80+5% Mannitol in H₂O; and

3% PEG 3350+0.3% Tween 80+5% Mannitol in 10 mM Phosphate Buffer, pH 7.4.

The suspensions showed good syringeability without sonication (some lump observed) using a 20G1″ Terumo needle and with 3 minutes sonication (some lumps observed) using a 22G1″ Terumo UTW needles in the vehicles as follows:

0.5% Plasdone C17+0.3% Tween 80 in H₂O; 0.5% Plasdone C17+0.3% Tween 80+5% Mannitol in H₂O; and 0.5% Plasdone C17+0.3% Tween 80+5% Mannitol in 10 mM Phosphate Buffer, pH 7.4.

The suspension in vehicle with 5% Mannitol in H₂O without Tween 80 and solubility enhancers, showed good syringeability without sonication (some lumps observed) using 20G1″ Terumo needle, and it was slightly difficult to withdraw after 3-minute sonication, using 22G1″ Terumo needle.

This study concluded that adding 5% Mannitol to the vehicles decreased the syringeability but adding 10 mM phosphate buffer at pH 7.4 showed no effect on the syringeability.

### Study #5 (Table 5)

This study evaluated the syringeability of 25HC3 S suspensions at 30 mg/mL, using Lot #A passed through a 20 mesh screen without jet milling. Study #4, used the same lot of 25HC3S, jet milled. The mixing method was manually shaking followed by sonication for 3 minutes (Mixing Method 2). The same vehicles were screened for Studies #4 and #5.

Without Jet milling, it showed good syringeability (one lump observed) with 22 G1″ Terumo needle for suspension in 3% PEG 3350+0.3% Tween 80 in H₂O and somewhat difficult or easy to withdraw but with lumps observed in the rest of vehicles.

Studies #4 and #5 concluded that 25HC3S, passed through a 20 mesh screen and jet-milled, showed best syringeability in 3% PEG 3350+0.3% Tween 80+5% Mannitol in 10 mM phosphate buffer, pH 7.4 with sonication (Mixing Method 2).

### Study #6 (Table 6)

This study evaluated the syringeability of 25HC3 S suspensions at 60 mg/mL, using Lot #A passed through a 20 mesh screen but not jet-milled in the same vehicles as those in study #5. Without jet milling and sonication, there were lumps observed. After 3-minute sonication, it showed good syringeability with 22 G1″ Terumo needle for suspension (with one lump observed) in 3% PEG 3350+0.3% Tween 80 in H₂O, and somewhat difficult or easy to withdraw but with lumps observed in the rest of vehicles.

Studies #5 and #6 concluded that there was no significant difference in syringeability between 30 or 60 mg/mL of 25HC3S suspensions.

### Study #7 (Table 7)

This study evaluated the syringeability of 25HC3S suspensions at 30 mg/mL in vehicles from study 6 with the addition of 0.15% L-Methionine, using Lot #B passed through a 20 mesh screen without jet milling. After being manually shaken 30 times, drug was hard to wet and sank at the bottom of the vial in 3% PEG 3350+0.3% Tween 80+0.15% L-Methionine in 10 mM phosphate buffer at pH 7.4 containing either 5% Mannitol or 0.9% NaCl. 25HC3S was not dispersed well in vehicles in 0.5% NaCMC+0.3% Tween 80+0.15% L-Methionine in 10 mM phosphate buffer at pH 7.4 containing either 5% Mannitol or 0.9% NaCl.

All formulation showed lumps and were difficult to withdraw with 20G1″ Terumo needle with or without 6-minute sonication.

Homogenization for 30 to 60 seconds produced suspensions that were easy to withdraw through 20G1″ to 22G1″ needles, with no particles remaining in the vials.

### Study #8 (Table 8)

This study compared the syringeability of 25HC3S (Lot #B), jet milled (Study #8) vs. not jet milled (study #7) in the same suspension vehicles at the same concentration of 30 mg/mL. The suspensions using jet-milled drug showed better syringeability.

### Study #9 (Table 9)

This study evaluated the effect of 0.1 and 0.2% NaCMC in suspension vehicles (to prevent the sedimentation) with 25HC3 S (Lot #A, delumped through 20 mesh screen followed by jet mill (3^{rd} pass). It was found at 30 mg/mL, 25HC3S was not completely dispersed well in 0.1% NaCMC, 3% PEG3350+0.3% Tween 80+5% Mannitol+0.15% L-Methionine in 10 mM Phosphate Buffer pH 7.4;

0.2% NaCMC, 3% PEG3350+0.3% Tween 80+5% Mannitol+0.15% L-Methionine in 10 mM Phosphate Buffer pH 7.4;

0.1% NaCMC, 3% PEG3350+0.3% Tween 80+0.9% NaCl+0.15% L-Methionine in 10 mM Phosphate Buffer pH 7.4; and 0.2% NaCMC, 3% PEG3350+0.3% Tween 80+0.9% NaCl+0.15% L-Methionine in 10 mM Phosphate Buffer pH 7.4.

All formed lumps after being manually shaken 30 times. After sonication for 6 minutes, it was still difficult to withdraw using a 20G1″ Terumo UTW needle.

### Study #10 (Table 10)

This study showed very good syringeability for 25HC3S Suspensions at 10 and 50 mg/mL in Vehicle PEG 3350 (with L-Methionine), prepared by homogenization using the drug without jet milling. The suspension can be withdrawn with a 25G⅝″ Terumo, UTW needle at 25HC3S concentration up 50 mg/mL. At 100 mg/mL, the suspension formed a thick paste that was unable to be withdrawn even using a 20G1″ Terumo, UTW needle.

### Study #11 (Table 11)

At 100 mg/mL, 25HC3S suspension in Vehicle PEG3350 (with L-Methionine) formed a thick paste. The syringeability was not tested. At 50 mg/mL, the suspension showed good syringeability, prepared by either homogenization or sonication, using 25HC3S (Lot #B), passed through a 20 mesh screen followed by jet milling 1^{st} pass. The suspension can be withdrawn with a 25G⅝″ Terumo, UTW needle. However, it was unable to know the exact volume due to foaming of suspension. When the vial was inverted, a few wet lumps stuck to the vial wall.

Based on Studies #10 and 11, 25HC3S suspension at 100 mg/mL in Vehicle PEG 3350 (with L-Methionine) formed a thick paste with poor syringeability. At 50 mg/mL, there were wet lumps stuck to the bottom or the side of the vial wall. Although the lumps had no effect on the syringeability, they might have effect on the homogeneity or label strength. Therefore, 25HC3 S suspension will be reduced to 25 mg/mL for future study.

### Study #12 (Table 12)

This study showed that 25HC3S at 25 mg/mL did not disperse well in Vehicle PEG 3350 (without L-Methionine) by shaking on a flat bed shaker at 100 rpm for up to 50 minutes. There were a few wet lumps stuck to the vial wall and the bottom of the vial. The wet lumps stuck to the vial wall and therefore, they did not affect the syringeability. However, they may have some effect on the homogeneity or % label strength.

### Study #13 (Table 13)

This study showed that 25HC3S at 25 mg/mL dispersed well in Vehicle PEG 3350 (without L-Methionine) by shaking on a flat bed shaker with a higher speed (200 rpm) for up to 45 minutes. Very few small wet lumps (compared to shaken at 100 rpm, Study #12) stuck to the vial wall. The suspension showed good syringeability.

Based on Studies #12 and 13, an improved shaking speed of 200 rpm on a flat bed shaker was chosen for Mixing Method 4.

### (B) Homogeneity Study

### Study #14 (Table 14)

This study showed good homogeneity (94.3-98.1% LS, 1.32% RSD, n=9) for 10 mg/mL of 25HC3S suspension in Vehicle PEG 3350 (with 0.15% L-Methionine and 0.9% NaCl). 25HC3S (Lot #B, Jet-milled, 3^{rd} pass) was used to prepare the suspension. The mixing method was manually shaking for 100 times followed by 30-minute sonication (Mixing Method 2). 1 mL each of the suspension (n=9, from the same 10 mL vial) was withdrawn using a 1 mL BD syringe attached to a 20G1″ Terumo UTW needle and dispensed for HPLC analysis. The less than 100% LS recovery may be due to that 25HC3 S (Lot #B), used for the suspension preparation, had lower purity, compared to 25HC3 S sodium salt (Lot #D) used for the external standard preparation. Both lots were not adjusted for peak purity.

### Study #15 (Table 15)

This study showed good homogeneity for 25HC3 S at 25 mg/mL (96.2-109.4% LS, 4.36% RSD, n=8, dispensed 0.2 mL each from the same 2 mL vial) and 25HC3S at 10 mg/mL (100.5-103.1% LS, 1.10% RSD, n=7, dispensed 0.9 mL each from the same 10 mL vial) in Vehicle PEG 3350 (with 0.15% L-Methionine), using a 25G⅝″ Terumo UTW needle and 1 mL BD syringe. The mixing method was manually shaken for 130 times followed by sonication for 30 minutes (Mixing Method 2). The suspension was prepared from 25HC3S (Lot #B, Jet-milled, 3^{rd} pass) and external standard was prepared from a mixed lot (Lot #E) for HPLC analysis.

### Study #16 (Table 16)

This study showed good homogeneity for 25HC3 S at 25 mg/mL suspended in Vehicle PEG 3350 (without 0.15% L-Methionine). The mixing method was shaken in a flat bed shaker at 100 rpm for 45 minutes (Mixing Method 4). After the preparation, the suspension was stored at room temperature. At each time point (time 0, 1, 2 and 19.5 hours), the suspension was inverted a few times and dispensed into HPLC vials at 100 μL each (n=2) and followed by 300 μL each (n=2), respectively for the homogeneity analysis, using a 25G⅝″ Terumo UTW needle and 1 mL BD syringe. The homogeneity ranged from 90.3 to 99.1% LS (n=8) for 100 μL samples and from 86.3 to 91.5% LS (n=8) for 300 μL samples. The lower % LS may be partially due to that the external reference standard (Lot #F) and suspension formulation (Lot #C, jet-milled and passed through 20 mesh screen) were prepared from two different lots. The standard was adjusted for peak purity but the suspension was not adjusted for peak purity. Some wet lumps stuck to the vial wall, were not withdrawn into the syringe for the sample dispensing for HPLC analysis. This also contributed to the lower % LS.

Based on studies 14-16, 25HC3S at 10 or 25 mg/mL, suspended in Vehicle PEG 3350 (with or without L-Methionine) showed good homogeneity (passed the acceptance criteria of 85-115% LS) with either Mixing Methods 2 or 4, using jet-milled drug.

### (C) Stability Study

### Studies #17-1 and 17-2 (Tables 17-1 and 17-2)

25HC3S suspension at 25 mg/mL in Vehicle PEG 3350 (with 0.15% L-Methionine) was stable for at least 2 weeks at ambient room temperature. After 2 weeks at room temperature (RT), the % peak area for 25HC3 S remained essentially unchanged at approximately 99.17% (using the peak area of 25HC3S plus two impurities as 100%, n=2, Table 17-2) with a drug potency of 103.7% (using time 0 concentration as 100%, n=2, Table 17-1). The main degradation products were the mixtures of 3β-Sulfate, 25-OH-5, 24-diene and 3β-Sulfate, 25-OH-5, 25-diene (RRT=2.6) and 25-OH Cholesterol (RRT=3.5).

### (D) Selection of Preferred Suspension Formulation for Improvement

Both 25HC3S suspensions at 25 mg/mL in Vehicle PEG3350 (with or without L-Methionine) showed good syringeability, prepared by Mixing Method 4 (Homogenization) with or without jet-milling the drug substance and by Mixing Method 2 and 4 (Manual shaking, followed by sonication or by mechanical shaking on a flat bed shaker at 200 rpm) with jet-milled drug.

The suspension showed good homogeneity and stability at room temperature (RT) for at least 2 weeks. However, after a long term storage (more than one month), the suspension with L-Methionine produced a sulfur-like odor which may be due to the degradation of L-Methionine. Therefore, L-Methionine was removed from Vehicle PEG 3350 for further improvement.

### (E) Improvement of the Preferred Formulation for Isotonicity Studies #18-1 and #18-2 (Tables 18-1 and 18-2)

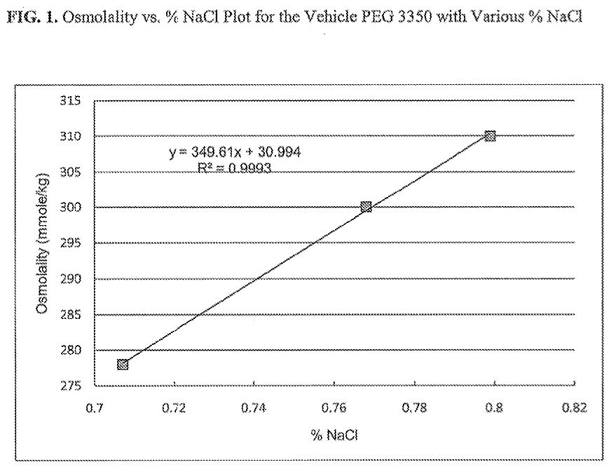

Table 18-1 summarizes the osmolality of the suspension vehicles (3% PEG 3350+0.3% Tween 80 in 10 mM phosphate buffer at pH 7.4) with 0.7 to 0.8% NaCl. The osmolality of the suspension vehicle at 0.75% NaCl was 293 mmol/kg, interpolated from the osmolality vs. % NaCl plot (FIG. 1). The solubility of 25HC3S was expected to be low in the vehicle such that 25HC3 S will not contribute too much to the osmolality value. Therefore, 25HC3S suspension at 25 mg/mL in this vehicle was expected to be close to the vehicle with isotonic solution (300 mmol/kg).

25HC3S at 25 mg/mL in 3% PEG 3350 plus 0.3% Tween 80 and 0.75% NaCl in 10 mM phosphate buffer at pH 7.4 was chosen as the final 25HC3S suspension formulation.

Table 18-2 summarizes the osmolality of placebo vehicle (Vehicle PEG 3350 without L-Methionine and with 0.75% NaCl) and 25HC3S Suspension formulation at 25 mg/mL in the placebo vehicle. The average osmolality of 6 consecutive measurements was 297 mmol/kg with a 0.3% RSD for the placebo vehicle and 321 mmol/kg with a 1.4% RSD for the 25HC3 S suspension formulation at 25 mg/mL.

(F) Homogeneity and Content Uniformity for Final 25HC3S Suspension Formulation at 25 mg/mL in 3% PEG 3350 plus 0.3% Tween 80 and 0.75% NaCl in 10 mM Phosphate Buffer at pH 7.4

### Study #19 (Table 19)

This study showed good homogeneity and content uniformity for 25HC3S. Suspension at 25 mg/mL in 3% PEG 3350 plus 0.3% Tween 80 and 0.75% NaCl in 10 mM phosphate buffer at pH 7.4. The homogeneity was determined by transferring 0.4 mL of suspension with 1 mL positive displacement pipet (n=6) and followed by transferring 0.4 mL suspension from the same vials with syringe attached to a needle (n=6). As listed in Table 19, the homogeneity and content uniformity as determined by HPLC ranged from 89.3 to 105.9% LS, 5.48% RSD, (n=6) for sample transferred with pipet and 98.2 to 100.4% LS, 0.96% RSD, (n=6) for sample transferred with syringe attached to a 27G½″ needle. The suspensions were prepared by Mixing Method 4 in a flat-bed shaker at 200 rpm for 45 minutes.

### CONCLUSION

25HC3S at 25 mg/mL, suspended in 3% PEG 3350 plus 0.3% Tween 80 and 0.75% NaCl in 10 mM phosphate buffer at pH 7.4 was chosen as the final suspension formulation. It showed good syringeability. The formulation was stable at room temperature for up to 14 days with a 99.172% 25HC3S by peak area normalization, essentially identical to that at Time 0. Mixing Method 3 (homogenization) showed the best physical appearance for the suspension with very few visible drug wet lumps. For long term stability and sterility purpose, a two-vial system was proposed. One vial was filled with 25HC3S powder (jet-milled) and the other vial was filled with Vehicle PEG 3350 (0.75% NaCl, no Methionine). The two vials were gamma irradiated. The desired volume of vehicle was withdrawn from the vial containing vehicle and added to the vial containing 25HC3 S powder and mixed in a flat bed mechanical shaker horizontally at 200 rpm for up to 45 minutes (Mixing Method 4). 25HC3S dispersed well in Vehicle PEG 3350 (0.75% NaCl, no Methionine) with very few lumps observed. The homogeneity and content uniformity ranged from 89.3 to 105.9% label strength, 5.48% RSD, by HPLC analysis (n=6, triplicate formulation preparations with duplicate injections for each preparation).

TABLE 1
Syringeability Study for 25HC3S Suspensions at 30 mg/mL in Various Vehicles
(Aqueous or Organic Solvents) by Manually Shaking without Sonication.
25HC35 (Lot# A) was Delumped through a 20 Mesh Screen With or Without Further Jet Milling
Mixing Method 1: Manually Shaking

| | | Mixing study | | Sedimentation | | Syringeability |
|---|---|---|---|---|---|---|
| | | | Appearance | after 1 min | | (1 mL BD |
| | Vehicle | Manual | of | at RT in | Needle | syringe, |
| 25HC3S | Composition | Mixing | suspension | 2 mL vial | size | luer lok) |
| Delump | 3% PEG | Shaken 15 | No lump | Not applicable | | |
| through 20 | 3350 + 0.05% | times | | | | |
| mesh screen | Tween | Shaken 30 | No lump | No | 20G 1″, | Easy to |
| only | 80 in H₂O | times | | sedimentation | Terumo | withdraw |
| | | | | | UTW | |
| | | | | | 21G1″, BD | Particles |
| | | | | | | clogged the |
| | | | | | | needle tip. |
| | | | | | | Discharge the |
| | | | | | | suspension |
| | | | | | | back to vial |
| | | | | | | and then could |
| | | | | | | re-withdraw |
| | | | | | | the suspension |
| | | | | | 22 G1″, BD | Same as 21G1″ |
| Pass through | | Shaken 15 | Some lumps | Not applicable | | |
| 20 mesh | | times | (or big | | | |
| screen then | | | particles) | | | |
| Jet-milled, 3^{rd} | | Shaken 30 | No lump | One lump settled | 20G 1″, | Easy to |
| Pass | | times | (No big | at the bottom | Terumo, | withdraw. |
| (deliberately | | | particles) | | UTW | However, one |
| chose big | | | | | | big lump left |
| agglomerates) | | | | | | at the bottom |
| for the study | | | | | | of vial |
| Pass through | | Shaken 15 | No lump | Not applicable | | |
| 20 mesh | | times | | | | |
| screen then | | Shaken 30 | No lump | No | 20G 1″, | Easy to |
| Jet-milled, 3^{rd} | | times | | sedimentation | Terumo | withdraw. No |
| Pass | | | | | UTW | lumps |
| (deliberately | | | | | 21G1″, BD | remaining |
| chose fine | | | | | | at bottom |
| particles) for | | | | | 22 G1″, BD | of vial |
| the study | | | | | | |
| Delump | 0.5% | Shaken 15 | Big | Not applicable | | |
| through 20 | NaCMC + | times | particles at | | | |
| mesh screen | 0.05% Tween | | the bottom | | | |
| | 80 in H₂O | | of the vial | | | |
| | | Shaken 30 | Big | No | 20G 1″ | OK, slightly |
| | | times | particles at | sedimentation | Terumo, | difficulty to |
| | | | the bottom | | UTW | withdraw, due |
| | | | | | | to some larger |
| | | | | | | particles |
| | | | | | 21G1″, BD | Same as above |
| | | | | | 22 G1″, BD | Same as above |
| | 0.25% | Shaken 15 | Big | Not applicable | | |
| | NaCMC + | times | particles at | | | |
| | 0.025% Tween | | the bottom | | | |
| | 80 in H₂O | | of the vial | | | |
| | | Shaken 30 | Big | No | 20G1.5″, BD | OK to withdraw, |
| | | times | particles at | sedimentation | | however some |
| | | | the bottom | | | small particles |
| | | | of the vial | | | left in the vial |
| | | | | | 21G1.5″, BD | Same as above |
| | | | | | 22 G1″, BD | Same as above |
| Delump | 0.9% NaCl in | Shaken 15 | No lump | Not applicable | | |
| through 20 | H₂O | times | | | | |
| mesh screen | | Shaken for | No lump | No | 20G 1″, | OK, one small |
| only | | 30 times | | sedimentation | Terumo, | particle clogged |
| | | | | | UTW | the needle |
| | | | | | 21G1″, BD | Difficult to |
| | | | | | | withdraw, |
| | | | | | | particles clogged |
| | | | | | | needle |
| | PG/H₂O = 50/50 | Shaken 15 | Particles at | Not applicable | | |
| | | times | the bottom | | | |
| | | Shaken 30 | Particles at | No | 20G 1″ | No problem to |
| | | times | the bottom | sedimentation | Terumo, | withdraw the |
| | | | | | UTW | suspension |
| | | | | | 21G1″, BD | Particles clogged |
| | | | | | | the needle |
| | Sesame oil | Shaken for | Particles at | Not applicable | | |
| | | 15 times | the bottom | | | |
| | | Shaken 30 | Particles at | Lots of particles | 20G 1″, | Cannot |
| | | times | the bottom | at the bottom | Terumo, | withdraw the |
| | | | | | UTW | suspension, |
| | | | | | | particles clogged |
| | | | | | | the needle |
| | Sesame | Shaken 15 | Particles at | Not applicable | | |
| | oil + 0.05% | times | the bottom | | | |
| | Tween 80 | Shaken 30 | Particles at | Lots of particles | 20G 1″ | Cannot |
| | | times | the bottom | at the bottom | Terumo, | withdraw the |
| | | | | | UTW | suspension, |
| | | | | | | particles clogged |
| | | | | | | the needle |
| | BA/BB = 10/90 | Shaken 15 | Particles at | Not applicable | | |
| | | times | the bottom | | | |
| | | Shaken for | Particles at | Lots of particles | 20G 1″, | Cannot |
| | | 30 times | the bottom | at the bottom | Terumo, | withdraw the |
| | | | | | UTW | suspension, |
| | | | | | | particles clogged |
| | | | | | | the needle |

TABLE 2
The Ease of Dispersion and Syringeability of 25HC3S Suspensions at 30 mg/mL after Sonication for Six Minutes
25HC35 (Lot# A) was Delumped through 20 Mesh Screen without Being Jet Milled
Mixing Method 2: Manually Shaken Followed by Sonication for 6 Minutes

| | | Manual Mixing | | Sedimentation | | | Syringeability |
|---|---|---|---|---|---|---|---|
| 25HC3S | | | Appearance | after 1 min | Sonication | | (1 mL BD |
| concentration | Vehicle | Mixing | of | at RT in | time and | Needle | syringe, |
| (mg/ml) | Composition | manually | suspension | 2 mL vial | appearance | size | luer lok) |
| 30 mg/mL | 3% PEG | Shaken 15 | Particles at | No | 6 minutes, | 20G 1″ | No difficulty |
| | 3350 + 0.05% | times | the bottom | sedimentation | visually | Terumo | to withdraw |
| | Tween 80 | Shaken 15 | No visible | | could not | 23G 1″ | No difficulty |
| | in water | times | particles at | | see any | Excel | to withdraw |
| | | again | the bottom, | | big lumps | 25G 5/8″ | No difficulty |
| | | | but some | | | BD | to withdraw |
| | | | particles | | | | |
| | | | around | | | | |
| | | | bottom edge | | | | |
| 30 mg/mL | 3% PEG | Shaken 15 | Some | No | 6 minutes, | 20G 1″ | No difficulty |
| | 3350 + 0.05% | times | particles | sedimentation | visually | Terumo | in |
| | Tween 80 + | | stuck on the | | could not | | withdrawing, |
| | 0.9% NaCl in | | wall of vial | | see any | | at the end, |
| | H₂O | | | | big lumps | | one lump |
| | | | | | | | stuck at the |
| | | | | | | | needle tip, |
| | | | | | | | discharged |
| | | | | | | | and withdraw |
| | | | | | | | again without |
| | | | | | | | difficulty |
| | | Shaken 15 | Some | | | 23G 1″ | One lump |
| | | times | particles | | | Excel | stuck at the |
| | | | stuck on the | | | | needle tip, |
| | | | wall of vial | | | | difficult |
| | | | | | | | to withdraw |
| | | | | | | 22G 1″ | One lump |
| | | | | | | BD | stuck at the |
| | | | | | | | needle tip, |
| | | | | | | | difficult |
| | | | | | | | to withdraw |
| 30 mg/mL | 3% PEG | Shaken 15 | Some | No | 6 minutes, | 20G 1″ | One lump |
| | 3350 + 0.9% | times | particles | sedimentation | visually | Terumo | stuck at the |
| | NaCl in H₂O | | stuck on the | | could not | | needle tip, |
| | | | wall and | | see any | | discharged |
| | | | bottom of | | big lumps | | and withdraw |
| | | | vial | | | | again without |
| | | | | | | | difficulty |
| | | Shaken 15 | Some | | | 22G 1 ″ BD | Big lump |
| | | times | particles | | | | stuck at the |
| | | | stuck on the | | | | needle tip, |
| | | | wall and | | | | difficult |
| | | | bottom of | | | | to withdraw |
| | | | vial | | | | |
| 30 mg/mL | 0.9% NaCl in | Shaken 15 | Some | No | 6 minutes, | 20G 1″ | One lump |
| | H₂O | times | particles | sedimentation | visually | Terumo | stuck at the |
| | | | stuck on the | | could not | | needle tip, |
| | | | wall of vial | | see any | | difficult |
| | | | | | big lumps | | to withdraw |
| | | Shaken 15 | Some | | | | |
| | | times | particles | | | | |
| | | | stuck on the | | | | |
| | | | wall of vial | | | | |
| 30 mg/mL | 0.5% | Shaken 15 | Some | A few particles | 6 minutes, | 20G 1″ | OK to |
| | Plasdone C17 + | times | particles | at the bottom | visually | Terumo | withdraw, but |
| | 0.9% NaCl in | | stuck on the | | could not | | at the end, |
| | H₂O | | wall of vial | | see any | | one lump |
| | | | | | big lumps | | stuck at the |
| | | | | | | | needle tip |
| | | Shaken 15 | Some | | | 22G 1 ″ BD | One lump |
| | | times | particles | | | | stuck at the |
| | | | stuck on the | | | | needle tip, |
| | | | wall of vial | | | | difficult |
| | | | | | | | to withdraw |

TABLE 3
The Ease of Dispersion and Syringeability of 25HC3S Suspensions at 100 mg/mL after Sonication for Six Minutes
25HC35 (Lot# A) was Delumped through 20 Mesh Screen Without Being Jet-Milled
Mixing Method 2: Manually Shaking Followed by Sonication for 6 Minutes

| | | Manual | | | | |
|---|---|---|---|---|---|---|
| | | Mixing and | Sedimentation | | | Syringeability |
| 25HC3S | | Appearance | after 1 min | Sonication | | (1 mL BD |
| concentration | Vehicle | of | at RT in | time and | Needle | syringe, |
| (mg/ml) | Composition | Suspension | 2 mL vial | appearance | size | luer lok) |
| 100 mg/mL | 3% PEG | Shaken 15 | Few particles | 6 minutes, | 20G 1″ | Initially OK to |
| | 3350 + 0.05% | times | on vial bottom | visually | Terumo | withdraw, but |
| | Tween 80 | Some | | could not | | then one big |
| | in water | particles | | see any | | lump stuck at |
| | | stuck on the | | big lumps | | needle tip, and |
| | | wall of vial | | | | difficult |
| | | | | | | to withdraw |
| | | Shaken 15 | | | 21G 1″ | One lump |
| | | times again | | | BD | stuck at the |
| | | | | | | needle tip, |
| | | | | | | difficult |
| | | | | | | to withdraw |
| 100 mg/mL | 3% PEG | Shaken 15 | Few particles | 6 minutes, | 20G 1″ | Difficult to |
| | 3350 + 0.05% | times | on vial bottom | visually | Terumo | withdraw. |
| | Tween 80 in | Lots of | | could not | | |
| | 0.9% NaCl | particles | | see any | | |
| | | stuck on the | | big lumps | | |
| | | wall of vial | | | | |
| | | Shaken 15 | | | | |
| | | times again | | | | |
| 100 mg/mL | 3% PEG | Shaken 15 | Some particles | 6 minutes, | 20G 1″ | Slightly difficult |
| | 3350 in | times | vial bottom | visually | Terumo | to withdraw. |
| | 0.9% NaCl | Some | | could not | | Disharge and |
| | | particles on | | see any | | re-withdraw, one |
| | | bottom of | | big lumps | | big lump stuck |
| | | vial | | | | at needle tip |
| | | Shaken 15 | | | | |
| | | times again | | | | |
| 100 mg/mL | in 0.9% NaCl | Shaken 15 | Particles stuck | 6 minutes, | 20G 1″ | Difficult to |
| | | times | on the wall | visually | Terumo | withdraw, big |
| | | A lot of | and bottom of | could not | | lump stuck |
| | | stuck on the | vial | see any | | at needle tip |
| | | wall and | | big lumps | | |
| | | bottom of vial | | | | |
| | | Shaken 15 | | | | |
| | | times again | | | | |
| 100 mg/mL | 0.5% | Shaken 15 | Particles stuck | 6 minutes, | 20G 1″ | Difficult to |
| | Plasdone C17 | times | on the wall | visually | Terumo | withdraw, big |
| | in 0.9% NaCl | A lot of | and bottom of | could not | | lump stuck |
| | | stuck on the | vial | see any | | at needle tip |
| | | wall and | | big lumps | | |
| | | bottom of vial | | | | |
| | | Shaken 15 | | | | |
| | | times again | | | | |

TABLE 4
Syringeability Study for 25HC3S Suspensions at 30 mg/mL
25HC3S (Lot# A) was Delumped through 20 Mesh Screen Followed by Jet
Milling (3rd pass) Mixing Method 2: Manually Shaken Followed by Sonication for 3 Minutes

| | Syingeability | Syringeability | | |
|---|---|---|---|---|
| | (prior to sonication) | (after 3 min sonication) | | |
| Vehicle | 20G1” needle | 20G1” needle | 21G1” needle | 22G1” needle |
| Composition | (Terumo, UTW) | (Terumo, UTW) | (Terumo, UTW) | (Terumo, UTW) |
| 3% PEG | Easy to withdraw | Easy to withdraw | Easy to withdraw | Easy to withdraw |
| 3350 + 0.3% | (No lump) | (No lump) | (No lump) | (No lump) |
| Tween 80 in | | | | |
| H₂O | | | | |
| 3% PEG | Easy to withdraw | Easy to withdraw | Easy to withdraw | Easy to withdraw |
| 3350 + 0.3% | (No lump) | (No lump) | (No lump) | (No lump) |
| Tween 80 +5% | | | | |
| Mannitol in | | | | |
| H₂O | | | | |
| 3% PEG | Easy to withdraw | Easy to withdraw | Easy to withdraw | Easy to withdraw |
| 3350 + 0.3% | (No lump) | (No lump) | (No lump) | (No lump) |
| Tween 80 + | | | | |
| 5% Mannitol + | | | | |
| 0.15% | | | | |
| Methionine in | | | | |
| 10 mM | | | | |
| Phosphate | | | | |
| Buffer, pH 7.4 | | | | |
| 5% Mannitol | OK to withdraw, | OK to withdraw, | Slightly difficult | Slightly difficult to |
| in H₂O | lumps stuck to | lumps left at the | to withdraw, | withdraw, lump did |
| | needle tip | bottom of the vial | lump did not | not block the needle |
| | | and on the wall | block the needle tip | tip |
| 0.5% Plasdone | Easy to withdraw | Easy to withdraw | Easy to withdraw | Easy to withdraw |
| C17 + 0.3% | | | (lump at bottom) | (No lump |
| Tween 80 in | | | | at bottom) |
| H₂O | | | | |
| 0.5% Plasdone | Easy to withdraw | Easy to withdraw | Easy to withdraw | Easy to withdraw |
| C17 + 0.3% | (No lump | (No lump at | (No lump at | (No lump |
| Tween 80 + | at bottom) | bottom) | bottom) | at bottom) |
| 5% Mannitol | | | | |
| in H₂O | | | | |
| 0.5% Plasdone | Easy to withdraw | Easy to withdraw | Easy to withdraw | Easy to withdraw |
| C17 + 0.3% | (No lump | (No lump at | (No lump at | (No lump |
| Tween 80 + | at bottom) | bottom) | bottom) | at bottom) |
| 5% Mannitol + | | | | |
| 0.15% | | | | |
| Methionine in | | | | |
| 10 mM | | | | |
| Phosphate | | | | |
| Buffer, pH 7.4 | | | | |

25HC3S was preweighed into the vial and capped with a stopper and stored at RT/3 days prior to syringeability study.
It was slightly difficult to disperse the drug in the vehicles (probably due to H2O absorption).
After manually shaking the suspension 45 times, 25HC3S was suspended well for the study.

TABLE 5
Effect of 25HC3S (Not Jet-Milled, Study #5) on the Syringeability of 25HC3S Suspension at
30 mg/mL 25HC3S (Lot# A) was delumped through a 20 Mesh Screen without Jet Milling
Mixing Method 2: Manually Shaken Followed by Sonication for 3 Minutes

| | Syringeability | | | |
|---|---|---|---|---|
| | (prior to | Syringeability | | |
| | sonication) | (after 3 min sonication) | | |
| | 20G1” needle | 20G1” needle | 21G1” needle | 22G1” needle |
| Vehicle | (Terumo, | (Terumo, | (Terumo, | (Terumo, |
| Composition | UTW) | UTW) | UTW) | UTW) |
| 3% PEG | Easy to | Easy to withdraw | Easy to withdraw | Easy to withdraw |
| 3350 + 0.3% | withdraw | (No lump | (No lump | but one lump stuck |
| Tween 80 in | (lumps left at | observed) | observed) | to needle tip |
| H₂O | the bottom of | | | in the middle of |
| | the vial) | | | withdrawing |
| 3% PEG | Easy to | Easy to withdraw | Easy to withdraw | Difficult to |
| 3350 + 0.3% | withdraw, | (some lumps left | (some lumps left | withdraw |
| Tween 80 + | particles | at the bottom of | at the bottom | (lump stuck to |
| 5% Mannitol | stuck to | the vial and | of the vial) | needle tip) |
| in H₂O | needle tip | on the wall) | | |
| 3% PEG | Easy to | Easy to withdraw, | Easy to withdraw | Easy to withdraw, |
| 3350 + 0.3% | withdraw, | particles stuck to | (lump stuck to | particles stuck to |
| Teen 80 + 5% | particles | needle tip at the | needle tip at the | needle tip in the |
| Mannitol in 10 | stuck to | end of | end of | middle of |
| mM Phosphate | needle tip | withdrawing | withdrawing | withdrawing |
| buffer pH 7.4 | | | | |
| 5% Mannitol | Difficult to | Easy to withdraw | Difficult to | Difficult to |
| in H₂O | withdraw, | (big lump left at | withdraw, lump | withdraw, |
| | particles | the bottom | stuck to needle | big lump stuck to |
| | stuck to | of the vial) | tip | needle tip |
| | needle tip | | | |
| 0.5% Plasdone | Easy to | Easy to withdraw | Slightly difficult | Difficult to |
| C17 + 0.3% | withdraw but | (lumps left at the | to withdraw, | withdraw, |
| Tween 80 in | lumps stuck | bottom of the vial) | lump stuck | lumps stuck to |
| H₂O | to needle tip | | to needle tip | needle tip |
| | at the end of | | | |
| | withdrawing | | | |
| 0.5% Plasdone | Easy to | Easy to withdraw, | Slightly difficult | Slightly difficult to |
| C17 + 0.3% | withdraw, | small lumps | to withdraw, | withdraw, lump |
| Tween 80 + | lumps left at | stuck to | lump stuck | stuck to |
| 5% Mannitol | the bottom of | needle tip | to needle tip | needle tip |
| in H₂O | the vial | | | |
| 0.5% Plasdone | Easy to | Easy to withdraw, | Easy to withdraw, | Easy to withdraw, |
| C17 + 0.3% | withdraw, | lumps left at the | lumps stuck to | lumps stuck to |
| Tween 80 + | small lumps | bottom of the vial | needle tip at the | needle tip at the |
| 5% Mannitol | stuck to | | end of | end of withdrawing |
| in 10 mM | needle tip at | | withdrawing | |
| Phosphate | the end of | | | |
| buffer, pH 7.4 | withdrawing | | | |

TABLE 6
Effect of 25HC3S (Not Jet-Milled, Study #6) on the Syringeability of 25HC3S Suspension at
60 mg/mL 25HC3S (Lot# A) was delumped through 20 Mesh Screen without Jet Milling
Mixing Method 2: Manually Shaken Followed by Sonication for 3 Minutes

| | Syringeability | | | |
|---|---|---|---|---|
| | (prior to | Syringeability | | |
| | sonication) | (after 3 min sonication) | | |
| | 20G1” needle | 20G1” needle | 21G1” needle | 22G1” needle |
| Vehicle | (Terumo, | (Terumo, | (Terumo, | (Terumo, |
| Composition | UTW) | UTW) | UTW) | UTW) |
| 3% PEG | Easy to | Easy to withdraw, no | Easy to withdraw, no | Easy to withdraw, no |
| 3350 + 0.3% | withdraw and | lumps at the bottom | lumps at the bottom | lumps at the bottom of the |
| Tween 80 in | discharge | of the vial | of the vial | vial |
| H₂O | | | | |
| 3% PEG | Easy to | OK to withdraw, at | OK to withdraw, no | Difficult to withdraw, |
| 3350 + 0.3% | withdraw, at | the end, one lump | lumps | lump stuck to needle tip |
| Tween 80 + 5% | the end, a | stuck to the needle | | |
| Mannitol in | piece of | tip | | |
| H₂O | particle stuck | | | |
| | to the needle | | | |
| | tip, but no | | | |
| | problem to | | | |
| | discharge | | | |
| 3% PEG | Easy to | OK to withdraw, | OK to withdraw, | OK to withdraw, small |
| 3350 + 0.3% | withdraw and | small lump at the | small lump at the | lump at the bottom of the |
| Tween 80 + 5% | discharge | bottom of the vial | bottom of the vial | vial |
| Mannitol + | | | | |
| 0.15% | | | | |
| Methionine in | | | | |
| 10 mM | | | | |
| Phosphate | | | | |
| buffer pH 7.4 | | | | |
| 5% Mannitol | Needle | OK to withdraw, | Slightly difficult to | Slightly difficult to |
| in H₂O | clogged, | lump stuck to needle | withdraw | withdraw |
| | discharge and | tip | | |
| | re-withdraw | | | |
| | are OK, small | | | |
| | lump at the | | | |
| | bottom of the | | | |
| | vial | | | |
| 0.5% Plasdone | Easy to | Easy to withdraw, | Slightly difficult to | Difficult to withdraw, |
| C17 + 0.3% | withdraw, | small lump at the | withdraw, lump at | lump stuck to needle tip |
| Tween 80 in | but big | bottom of the vial | needle tip | |
| H₂O | lumps at the | | | |
| | bottom edge | | | |
| | of the vial, | | | |
| | discharge and | | | |
| | re-withdraw | | | |
| | slightly | | | |
| | difficulty | | | |
| 0.5% Plasdon | Easy to | Easy to withdraw, | OK to withdraw, | OK to withdraw, lump |
| C17 + 0.3% | withdraw, | but one lump at the | lump stuck to the | stuck to the needle tip |
| Tween 80 + 5% | but a small | bottom of the vial | needle tip | |
| Mannitol in | lump at the | | | |
| H₂O | bottom of the | | | |
| | vial | | | |
| 0.5% Plasdon | Easy to | Easy to withdraw, at | Easy to withdraw, | Difficult to withdraw, |
| C17 + 0.3% | withdraw, | the end of withdraw, | but see a big lump at | lump stuck to the needle |
| Tween 80 + 5% | but a small | a piece of lump stuck | the bottom of vial | tip |
| Mannitol + | lump at the | to needle tip. | | |
| 0.15% | bottom of the | Discharge and re- | | |
| Methionine in | vial | withdraw no problem | | |
| 10 mM | | | | |
| Phosphate | | | | |
| Buffer, pH 7.4 | | | | |

TABLE 7
Syringeability Study for 25HC3S Suspensions at 30 mg/mL
25HC3S (Lot# B) was Delumped through 20 Mesh Screen Without jet -Milling
Mixing Method 2 or 3: Manually Shaken Followed by Sonication for 6 Minutes or
Homogenized 30-60 Seconds

| | Mixing Study | | Syringeability | | |
|---|---|---|---|---|---|
| | Manual | Appearance of | | Prior to | After 6 minutes |
| Vehicle composition | Shaking | Suspension | Needle size | sonication | sonication |
| 3% PEG 3350 + 0.3% | Shaken 15 | Hard to wet, | 20G1″ Terumo | Easy to | Difficult to |
| Tween 80 + 5% | times | particles on the | | withdraw but | withdraw, lumps |
| mannitol + 0.15% L- | | vial wall, sank | | lots of | stuck to needle |
| Methionine in 10 mM | | at the bottom | | particles left | tip. Particles |
| Phosphate Buffer at | Shaken 15 | Hard to wet, | | at the bottom | sank at the |
| pH 7.4 | times again | particles sank | | | bottom |
| | | at the bottom | | | |
| 3% PEG 3350 + 0.3% | Shaken 15 | Lots of | | Difficult to | Difficult to |
| Tween 80 + 0.9% | times | particles at the | | withdraw, | withdraw. |
| NaCl + 0.15% | Shaken 15 | bottom | | particles | Particles sank at |
| L-Methionine in 10 | times again | | | stuck to | the bottom |
| mM Phosphate Buffer | | | | needle tip | |
| at pH 7.4 | | | | | |
| 0.5% NaCMC + 0.3% | Shaken 15 | 25HC3S not | | Not tested, | Difficult to |
| Tween 80 +5 % | times | dispersed well | | Drug not | withdraw. |
| mannitol + 0.15% L- | Shaken 15 | | | dispersed | Particles sank at |
| Methionine in 10 mM | times again | | | well | the bottom with |
| Phosphate Buffer at | | | | | some particles |
| pH 7.4 | | | | | floating in the |
| | | | | | middle |
| 0.3% Tween 80 + | Shaken 15 | Hard to wet, | | Difficult to | Difficult to |
| 0.9%NaCl + 0.15% | times | particles sank | | withdraw, | withdraw. |
| L-Methionine in 10 | Shaken 15 | at the bottom | | particles | Particles sank at |
| mM Phosphate Buffer | times again | | | stuck to | the bottom |
| at pH 7.4 | | | | needle tip | |

| | | | | |
|---|---|---|---|---|
| 3% PEG 3350 + 0.3% | Homogenized | Uniform | 20G1″ Terumo | Easy to withdraw (no particles) |
| Tween 80 + 5% | 30 seconds | | 21G1″ Terumo | Easy to withdraw (no particles) |
| mannitol + 0.15% L- | (no manual | | 22G1″ Terumo | Easy to withdraw (no particles) |
| Methionine in 10 mM | shaking) | | | |
| Phosphate Buffer at | | | | |
| pH 7.4 | | | | |
| 0.5% NaCMC + 0.3% | Homogenized | Uniform | 20G1″ Terumo | Easy to withdraw (no particles) |
| Tween 80 + 5% | 60 seconds | | 21G1″ Terumo | OK to withdraw (no particles) |
| mannitol + 0.15% L- | (no manual | | 22G1″ Terumo | OK to withdraw (no particles) |
| Methionine in 10 mM | shaking) | | | |
| Phosphate Buffer at | | | | |
| pH 7.4 | | | | |

TABLE 8
Syringeability Study for 25HC3S Suspensions at 30 mg/mL
25HC3S (Lot# B) was delumped through a 20 Mesh Screen and Jet-Milled (1st pass)
Mixing Method 2: Manually Shaken and Followed by Sonication for 6 Minutes

| | Mixing Study | | | |
|---|---|---|---|---|
| | Manual | Appearance of | Syringeability | |
| Vehicle composition | Shaking | Suspension | Needle size | After 6 minutes sonication |
| 3% PEG 3350 + 0.3% | Shaken 15 | Hard to wet, | 20G1”, 21G1” | Easy to withdraw |
| Tween 80 + 5% | times | particles on the | and 22G1” | |
| mannitol + 0.15% L- | | vial wall, sank | Terumo, UTW | |
| Methionine in 10 mM | | at the bottom | | |
| Phosphate Buffer at | Shaken 15 | Hard to wet, | | |
| pH 7.4 | times again | particles sank at | | |
| | | the bottom | | |
| 3% PEG 3350 + 0.3% | Shaken 15 | Lots of particles | | Easy to withdraw |
| Tween 80 + 0.9% | times | at the bottom | | |
| NaCl + 0.15% | Shaken 15 | | | |
| L-Methionine in 10 | times again | | | |
| mM Phosphate Buffer | | | | |
| at pH 7.4 | | | | |
| 0.5% NaCMC + 0.3% | Shaken | 25HC3S not | 20G1” Terumo, | Easy to withdraw |
| Tween 80 + 5% | 30times | dispersed well | UTW | with a few |
| mannitol + 0.15% L- | | Particles all | | Particles sank |
| Methionine in 10 mM | | over the vial | | at the bottom |
| Phosphate Buffer at | | | | |
| pH 7.4 | | | | |

TABLE 9
Effect of NaCMC on the Syringeability of 25HC3S Suspension at 30 mg/mL
25HC3S (Lot#A) Was Delumped through 20 Mesh Screen Followed by jet milling (3^{rd} pass)
Mixing Method 2: Manually Shaken Followed by Sonication for 6 Minutes

| | Mixing study | | Sonication | | Syringeability |
|---|---|---|---|---|---|
| Vehicle | Mixing | Appearance of | time and | | (1 mL BD |
| Composition | manually | suspension | appearance | Needle size | syringe, luer lok) |
| 0.1% NaCMC, | Shaken for | Lots of particles | 6 minutes, | 20G1″ | No difficulty to |
| 3% PEG 3350 + | 15 times | and lumps on | quite a few | Terumo | withdraw, but lots |
| 0.3% Tween 80 + | | the wall and at | particles and | | of lumps left at |
| 5% Mannitol + | | the bottom | some lumps | | the bottom |
| 0.15% L- | Shaken for | Same as above, | | | |
| Methionine in 10 | 15 times | particles and | | | |
| mM Buffer pH | again | lumps on the | | | |
| 7.4 | | wall and at the | | | |
| | | bottom | | | |
| 0.2% NaCMC, | Shaken for | Lots of particles | 6 minutes, | 20G1″ | No difficulty to |
| 3% PEG 3350 + | 15 times | and lumps on | quite a few | Terumo | withdraw, but lots |
| 0.3% Tween 80 + | | the wall and at | particles and | | of lumps left at |
| 5% Mannitol + | | the bottom | some lumps | | the bottom |
| 0.15% L- | Shaken for | Same as above, | | | |
| Methionine in10 | 15 times | particles and | | | |
| mM Buffer pH | again | lumps on the | | | |
| 7.4 | | wall and at the | | | |
| | | bottom | | | |
| 0.1% NaCMC, | Shaken for | Lots of particles | 6 minutes, | 20G1″ | No difficulty to |
| 3% PEG 3350 + | 15 times | and lumps on | some particles | Terumo | withdraw, but |
| 0.3% Tween 80 + | | the wall and at | or small lumps | | small lump left at |
| 0.9% NaCl + | | the bottom | | | the bottom |
| 0.15% L- | Shaken for | Same as above, | | | |
| Methionine in10 | 15 times | particles and | | | |
| mM Buffer pH | again | lumps on the | | | |
| 7.4 | | wall and at the | | | |
| | | bottom | | | |
| 0.2% NaCMC, | Shaken for | Lots of particles | 6 minutes, | 20G 1″ | No difficulty to |
| 3% PEG 3350 + | 15 times | and lumps on | quite a few | Terumo | withdraw, but lots |
| 0.3% Tween 80 + | | the wall and at | particles and | | of lumps left at |
| 0.9% NaCl + | | the bottom | some lumps | | the bottom |
| 0.15% L- | Shaken for | Same as above, | | | |
| Methionine in10 | 15 times | particles and | | | |
| mM Buffer pH | again | lumps on the | | | |
| 7.4 | | wall and at the | | | |
| | | bottom | | | |

TABLE 10
Syringeability for 25HC3S Suspensions at 10, 50 and 100 mg/mL in Vehicle PEG 3350 (with L-Methionine)
25HC3S (Lot# B), Passed through 20 Mesh Screen but Not Jet-Milled
Mixing Method 3: Homogenization with PowerGen 1000 attached to a 5 × 95 mm Probed at
Speed Setting of 4 for 30 Seconds

| | | | | 1 mL BD | | | |
|---|---|---|---|---|---|---|---|
| | | | | syringe with | | | |
| Vial | | | | Terumo | Syringeability | | |
| (2 mL) | 25HC35 | Vehicle | Vehicle | needle | | 1 hr after | 3 hrs after |
| # | (mg/mL) | composition | (mL) | UTW | Time 0 | Homogenization | Homogenization |
| 1 | 11.1 | 3% PEG | 1 | 25G5/8″ | Easy to | Easy to | Easy to |
| | | 3350 + 0.7% | | | withdraw | withdraw | withdraw |
| 2 | 50.4 | NaCl + 0.3% | 1 | 25G5/8″ | Easy to | Formed thick | Formed thick |
| | | Tween | | | withdraw | paste, manually | paste, manually |
| | | 80 + 0.15% | | | | shake the vial, | shake the vial, |
| | | L-Methionine | | | | easy to withdraw | easy to withdraw |
| 3 | 99.8 | in 10 mM | 1 | 25G5/8″, | Unable to | Unable to | Unable to |
| | | Phosphate at | | 22G ″ and | withdraw, | withdraw | withdraw |
| | | pH 7.4 | | 20G1″ | formulation | | |
| | | | | | formed | | |
| | | | | | thick Paste | | |

TABLE 11
Syringeability for 25HC3S Suspensions at 50 and 100 mg/mL in Vehicle PEG 3350 (with L-Methionine)
25HC35 (Lot# B), Passed through 20 Mesh Screen Followed by Jet-Milled, 1st pass
Mixing Methods: Manual Shaking Followed by Sonication (Mixing Method 2) or
Homogenization only (Mixing Method 3)

| | | | Syringeability | | | |
|---|---|---|---|---|---|---|
| | | | | 23G1″ | | |
| | | | 22G1″ UTW | UTW | 25G5/8″ | |
| 25HC3S | | | Terumo | Terumo | UTW | |
| (mg/mL) | Mixing Method | Appearance | Surguard 3 | Surguard 3 | Terumo | Comments |
| 50 | Sonication 30 | Milky thick | Easy to | Easy to | Easy to | Formulation was thick, |
| | min, shake the | suspension | withdraw | withdraw | withdraw | after flip over the vial |
| | vial 3 times | | and dispense | and | and | for withdraw, some |
| | every 5 min | | | dispense | dispense | suspension was stuck |
| | during the | | | | | on the wall or at the |
| | sonication | | | | | vial bottom. |
| 50 | Homogenization | Milky thick | Easy to | Easy to | Easy to | Suspension was also |
| | for 30 seconds | suspension | withdraw | withdraw | withdraw | foaming, it was very |
| | | | and dispense | and | and | hard to tell how much |
| | | | | dispense | dispense | volume was withdrawn |
| | | | | | | in the syringe. |
| 100 | Homogenization | White paste | Not | NA | NA | NA |
| | for 60 seconds | | flowable, | | | |
| | | | not able to | | | |
| | | | withdraw | | | |
| | | | anything | | | |

TABLE 12
Syringeability for 25HC3S Suspensions at 25 mg/mL in 3% PEG 3350 + 0.3% Tween 80 + 0.7%
NaCl in 10 mM Phosphate Buffer at pH 7.4,
25HC3S (Lot# C), (Jet Milled, With or Without Further Passing Through a 20 Mesh Screen)
Mixing Method 4: Shaken at 100 rpm Horizontally on a Flat Bed Shaker for Approximately 45
Minutes

| | | | | | | Syringeability |
|---|---|---|---|---|---|---|
| | | | | | | (withdraw 0.9 |
| | Suspension | | | | | mL and |
| | Formulation | Flat bed | Appearance of | | | discharge 0.9 |
| 25HC3S | vial | shaker | suspension | Needle | Syringe | mL) |
| Jet-Milled | 1 | 100 rpm/ | Some wet | Terumo | 1 mL BD | Easy to |
| only | | 45 min | lumps at the | 23G1″, UTW | | withdraw and |
| | | | bottom and on | Terumo | | easy to |
| | | | the side of the | 25G5/8″, | | discharge |
| | | | vial wall | UTW | | |
| | | | | Exel | | |
| | | | | 27G11/2″ | | |
| Jet-Milled | 2 | 100 rpm/ | A few wet | Terumo | 1 mL BD | Easy to |
| and Passed | | 45 min | lumps at the | 23G1″, UTW | | withdraw and |
| Through a | | | bottom and on | Terumo | | easy to |
| 20 Mesh | | | the side of the | 25G5/8″, | | discharge |
| Screen | | | vial wall | UTW | | |
| | | | | Exel | | |
| | | | | 27G11/2″ | | |
| Jet-Milled | 3 | 100 rpm/ | A few particles | 25Gx1″ | 1 mL BD | Easy to |
| and then | | 50 min | (actually very | 26Gx3/8″ | | withdraw and |
| Passed | | | small wet | 26Gx 1/2″ | | easy to |
| Through a | | | lumps at the | 27Gx1/2″ | | discharge |
| 20 Mesh | | | bottom and on | | | |
| Screen | | | the side of the | 27Gx1/2″ | BD-1/2 mL | Easy to |
| | | | vial wall) | | tuberculin | withdraw and |
| | | | | | syringe | discharge |

TABLE 13
Syringeability for 25HC3S Suspensions at 25 mg/mL in 3% PEG 3350 +0.3% Tween 80
+0.7% NaCl in 10 mM Phosphate Buffer at pH 7.4 25HC3S (Lot# C, Jet Milled without
Passing through a 20 Mesh Screen first),
Mixing Method 4: Shaken at 200 rpm Horizontally on a Flat Bed Shaker at Various Time
Intervals

| | | | | | Syringeability (withdraw |
|---|---|---|---|---|---|
| | | | | | 0.5 mL and discharge 0.5 |
| | Suspension | | | | mL to a clean vial) in |
| | Formulation | Flat bed | Appearance of | | Quintuplicate with the |
| 25HC35 | vial # | shaker | suspension | Syringe | same syringe |
| Micronized | 1 | 200 | A few wet lumps | BD-1/2mL | Easy to withdraw and |
| only (not | | rpm/15 | observed | tuberculin | dispense |
| passed | | min | | syringe | |
| through 20 | 2 | 200 | Very small | attached to a | Easy to withdraw and |
| mesh screen) | | rpm/30 | amount of wet | 27Gx1/2″ | dispense |
| | | min | lumps observed | needle | |
| | 3 | 200 | One or 2 wet | | Easy to withdraw and |
| | | rpm/45 | lumps observed | | dispense |
| | | min | | | |

TABLE 14
Homogeneity¹ for 25HC3S at 10 mg/mL Suspension in Vehicle PEG 3350 (with 0.15% L-
Methionine and 0.9% NaCl) by HPLC 25HC35 (Lot# B, Passed through a 20 Mesh Screen
and Jet-Milled, 3^{rd} Pass)
Mixing Method 2: Manually Shaken for 100 times Followed by Sonication for 6 Minutes
(Suspension Was Prepared and Stored at RT for 5 Days and Re-Suspended for HPLC Analysis)

| Sample # | Sample conc. (mg/mL)² | % Label Strength (% LS) |
|---|---|---|
| S-1 | 9.683 | 96.8 |
| S-2 | 9.627 | 96.3 |
| S-3 | 9.471 | 94.7 |
| S-4 | 9.582 | 95.8 |
| S-5 | 9.431 | 94.3 |
| S-6 | 9.610 | 96.1 |
| S-7 | 9.511 | 95.1 |
| S-8 | 9.761 | 97.6 |
| S-9 | 9.807 | 98.1 |
| S1-S9 | Average = 9.609 | Range; 94.3 to 98.1% LS |
| | SD = 0.127 | |
| | % RSD = 1.32 | |

¹The suspension was dispensed through 20G1″ Terumo needle attached to a 1 mL BD syringe. Total of 9 samples, each with 1 mL suspension from the same 10 mL vial were dispensed for the homogeneity study. The suspension was slightly hazy. No centrifugation prior to HPLC analysis.
²The concentration was obtained by HPLC using Lot# D as external standard for HPLC analysis. The suspension was prepared with Lot# B, which showed lower potency compared to Lot# D, which was used as reference standard.

TABLE 15
Homogeneity for 25HC3S, 10 and 25 mg/mL Suspension in 3% PEG 3350 +0.3% Tween 80
+0.7% NaCl +0.15% L-Methionine in 10 mM Phosphate Buffer at pH 7.4 by HPLC 25HC35
(Lot# B, Passed through 20 Mesh Screen and Jet-Milled, 3^{rd} Pass)
Mixing Method 2: Manually Shaken for 100 times Followed by Sonication for 6 Minutes (the
Suspension was Stored at RT/5 Days Prior to HPLC Analysis)

| | | | Sample Concentration | % Label Strength |
|---|---|---|---|---|
| Sample Concentration | | Sample ID | (mg/mL) | (% LS) |
| 25.19 | mg/mL | S-1 | 25.26 | 100.3 |
| | | S-2 | 25.81 | 102.5 |
| | | S-3 | 24.24 | 96.2 |
| | | S-4 | 26.13 | 103.7 |
| | | S-5 | 25.43 | 101.0 |
| | | S-6 | 24.25 | 96.3 |
| | | S-7 | 24.70 | 98.1 |
| | | S-8 | 27.57 | 109.4 |
| | | S-1 to S-8 | Average = 25.42 | Range 96.2 to 109.4 |
| | | | SD = 1.11 | |
| | | | %RSD = 4.36 | |
| 9.985 | mg/mL | S-1 | 10.14 | 101.6 |

| | | |
|---|---|---|
| S-2 | 10.23 | 102.5 |
| S-3 | 10.21 | 102.3 |
| S-4 | 10.28 | 103.0 |
| S-5 | 10.02 | 100.4 |
| S-6 | 10.29 | 103.1 |
| S-7 | 10.03 | 100.5 |
| S1-S7 | Average = 10.17 | Range 100.5 to 103.1 |
| | SD = 0.11 | |
| | % RSD = 1.10 | |

Mixed lot (Lot #E) was used as external standard for HPLC analysis. The suspension was prepared with Lot #B.

TABLE 16
Homogeneity for 25HC3S at 25 mg/mL suspended in 3% PEG 3350 plus 0.3% Tween 80, and 0.7%
NaCl in 10 mM Phosphate Buffer at pH 7.4 25HC3S (Lot# C, Jet Milled Followed by Passing
through a 20 Mesh Screen)
Mixing Method 4: Placed in a Flat Bed Shaker at 100 RPM for 45 Minutes

| Suspension | | | | | | % Label |
|---|---|---|---|---|---|---|
| volume- | Appearance of | Dispensed time after | | Potency (mg/mL)¹ | | strength² |
| Sample name | suspension | formulation prep | | at UV 220 nm | at UV 205 nm | at UV 205 nm |
| 100 μL-S1 | Milky suspension | Time 0 | | 23.32 | 22.93 | 90.3 |
| 100 μL-S2 | with some particles | | | 24.43 | 24.18 | 95.2 |
| 100 μL-S3 | observed | 1 | Hour/RT | 25.39 | 25.16 | 99.0 |
| 100 μL-S4 | | | | 25.27 | 25.09 | 98.8 |
| 100 μL-S5 | | 2 | Hours/RT | 25.43 | 25.17 | 99.1 |
| 100 μL-S6 | | | | 25.36 | 25.16 | 99.0 |
| 100 μL-S7 | Milky suspension | 19.5 | Hours/RT | Not determined | 24.74 | 97.4 |
| 100 μL-S8 | with no particle | | | Not determined | 24.58 | 96.8 |
| | observed | | | | | |
| NA | | Average (n = 6 or 8)) | | 24.87 | 24.63 | Range: 90.3- |
| | | Std dev (n = 6 or 8) | | 0.85 | 0.77 | 99.1% LS |
| | | % RSD (n = 6 or 8) | | 3.40 | 3.13 | |
| 300 μL-S1 | Milky suspension | time 0 | | 22.37 | 21.93 | 86.3 |
| 300 μL-S2 | with some particles | | | 22.61 | 22.17 | 87.3 |
| 300 μL-S3 | observed | 1 | Hour/RT | 23.57 | 23.25 | 91.5 |
| 300 μL-S4 | | | | 23.67 | 23.18 | 91.3 |
| 300 μL-S5 | | 2 | Hours/RT | 23.42 | 22.94 | 90.3 |
| 300 μL-S6 | | | | 22.96 | 22.41 | 88.2 |
| 300 μL-S7 | Milky suspension | 19.5 | Hours/RT | Not determined | 22.57 | 88.9 |

| | | | | |
|---|---|---|---|---|
| 300 μL-S8 | with no particle | Not determined | 23.02 | 90.6 |
| | observed | | | |

| | | | | |
|---|---|---|---|---|
| NA | Average (n = 6 or 8) | 23.10 | 22.68 | Range: 86.3- |
| | Std dev (n = 6 or 8) | 0.54 | 0.49 | 91.5% |
| | % RSD (n = 6 or 8) | 2.32 | 2.15 | |

¹The concentration was obtained by HPLC using Lot# F as external reference standard (adjusted for 95.8% purity) for HPLC analysis. The suspension was prepared with Lot# C and was not adjusted for peak purity. Therefore, the % Label strength was lower than expected.
²Target concentration was 25.4 mg/mL.

TABLE 17-1
Stability¹ for 25HC3S Suspension at 25 mg/mL in 3% PEG 3350 +0.3%
Tween 80 +0.7% NaCl +0.15% L-Methionine in 10 mM Phosphate Buffer
at pH 7.4 by HPLC 25HC3S (Lot# B, Passed through 20 mesh screen and
Jet-Milled, 3^{rd} Pass)
Mixing Method 2: Manually Shaken for 30 Times Followed by Sonication
for 6 Minutes

| | | | % Drug remaining |
|---|---|---|---|
| | 25HC35 Concentration (mg/mL) at | | after 2 weeks at |
| Sample ID¹ | Time 0 | RT/2 weeks | RT |
| 1-1 | 26.40 | 26.12 | |
| 1-2 | 25.40 | 27.62 | |
| average | 25.9 | 26.87 | 103.7 |

¹The same sample suspension from Study#14

TABLE 17-2
Impurity Profile¹ for 25HC3S Suspension at 25 mg/mL in 3% PEG
3350 +0.3% Tween 80 +0.7% NaCl +0.15% L-Methionine in 10
mM Phosphate Buffer at pH 7.4 by HPLC 25HC3S (Lot# B, Passed
through a 20 Mesh Screen and Jet-Milled, 3^{rd} Pass)
Mixing Method 2. Manually Shaken for 100 Times Followed by
Sonication for 6 Minutes

| | % Peak area (n = 2) at | | |
|---|---|---|---|
| | RRT = 2.6 | | |
| | (mixture of 3β- | | |
| | Sulfate,25-OH-5, | | |
| | 24-diene and 3β- | RRT = 3.5 | |
| | Sulfate,25-OH-5, | (25-OH | RRT = 1 |
| Time point | 25-diene) | Cholesterol) | (25HC3S) |
| Initial (time 0) | 0.514 | 0.321 | 99.166 |
| RT/2 weeks | 0.508 | 0.320 | 99.172 |

¹The same sample suspension from Study#14

TABLE 18-1
Osmolaity for Vehicle PEG 3350 (3% PEG 3350 plus 0.3% Tween
80 in 10 mM PhosphateBuffer at pH 7.4) Containing Various %
NaCl, Measured by a Vapor Pressure Osmometer

| % NaCl in the suspension Vehicle | Osmolality (mmol/kg) |
|---|---|
| 0.707 | 278 |
| 0.768 | 300 |
| 0.799 | 310 |

TABLE 18-2
Osmolaity for the Improved Vehicle PEG 3350 and Final Improved
25HC3S Suspension Formulation at 25 mg/mL, Measured by a Vapor
Pressure Osmometer 25HC3S (Lot# B, Jet-milled, one pass)

| | Osmolality (mmol/kg) | |
|---|---|---|
| | | 25HC3S Suspension at |
| Run# | Vehicle PEG 3350 | 25 mg/mL |
| 1 | 298 | 316 |
| 2 | 297 | 322 |
| 3 | 296 | 320 |
| 4 | 298 | 319 |
| 5 | 297 | 329 |
| 6 | 298 | 322 |
| Average Osmolality | 297 | 321 |
| (n = 6) | | |
| Std dev (n = 6) | 0.8 | 4.4 |
| % RSD | 0.3 | 1.4 |

TABLE 19
Homogeneity and Content of Uniformity of 25HC3S Suspension at 25 mg/mL in 3% PEG 3350
plus 0.3% Tween 80 and 0.75% NaCl in 10 mM Phosphate Buffer at pH 7.4 by HPLC
25HC35 (Lot# B, Jet milled, One Pass)
Mixing Method 4: Placed Horizontally in a Flat Bed Shaker, Shaken at 200 rpm for 45 Minutes

| | | | % recovery | | | | % |
|---|---|---|---|---|---|---|---|
| | 0.4 mL | Appearance of | (actual/ | | | | Recovery |
| Sample | suspension | suspension in | theoretical) | | std dev | % RSD | range |
| ID | transfer method | MeOH | n = 1 | n = 6 | n = 6 | n = 6 | n = 6 |
| vial 1-1 | 1 mL positive | Invert the flask | 98.93 | 97.45 | 5.34 | 5.48 | 89.3-105.9 |
| vial 1-2 | displacement | to dissolve the | 97.77 | | | | |
| | pipet | drug | | | | | |
| vial 2-1 | | Lots of | 105.88 | | | | |
| vial 2-2 | | 25HC3S | | | | | |
| vial 3-1 | | granules, need | | | | | |
| vial 3-2 | | to be vortexed | | | | | |
| | | to dissolve | | | | | |
| | | Invert the flask | 96.03 | | | | |
| | | to dissolve the | 89.28 | | | | |
| | | drug | 96.83 | | | | |
| vial 1-3 | 1 mL BD syringe | Invert the flask | 98.37 | | | | |
| Vial 1-4 | attached to a BD | to dissolve the | 100.08 | | | | |
| vial 2-3 | 27G1/2″ needle | drug | 98.21 | | | | |
| Vial 2-4 | | | 98.6 | | | | |
| vial 3-3 | | | 100.35 | | | | |
| Vial 3-4 | | | 99.87 | 99.25 | 0.96 | 0.96 | 98.2-100.4 |

### Example 2B. Oral Formulations

The below oral formulations were made as follows. Elevated temperatures ranging from about 50° C. to 70° C. were used to readily liquefy the Gelucire. The other excipients and 25HC3 S sodium salt were added with stirring. While the formulation was still warm, it was filled into capsules.

Examples of capsule formulations in which we have in-vitro dissolution data include:
1. 30% (w/w) drug and 70% (w/w) Gelucire 44/14-150 mg drug/capsule
2. 30% (w/w) drug and 70% (w/w) Gelucire 50/13-150 mg drug/capsule
3. 30% (w/w) drug and 35% (w/w) Gelucire 44/14 and 35% (w/w) PEG-400-150 mg drug/capsule
4. 30% (w/w) drug and 32.5% (w/w) Gelucire 44/14 and 32.5% (w/w) PEG-400 and 5% (w/w) methocel E3-150 mg drug/capsule
5. 10% (w/w) drug and 90% (w/w) Gelucire 44/14-50 mg drug/capsule
6. 10% (w/w) drug and 85% (w/w) Gelucire 44/14 and 5% (w/w) Ac-Di-Sol—50 mg drug/capsule
7. 10% (w/w) drug and 42.5% (w/w) Gelucire 44/14 and 42.5% (w/w) PEG-400 and 5% (w/w) Ac-Di-Sol—50 mg drug/capsule
8. 20% (w/w) drug and 70% (w/w) Gelucire 44/14 and 10% (w/w) Ac-Di-Sol—100 mg drug/capsule
9. 14.3 drug and 50% (w/w) Gelucire 44/14 and 28.6% (w/w) PEG-400 and 7.1% (w/w) Ac-Di-Sol—50 mg/capsule
10. 15% (w/w) drug and 40% (w/w) Gelucire 44/14 and 40% (w/w) PEG-400 and 5% (w/w) Ac-Di-Sol—100 mg drug/capsule
11. 15% (w/w) drug and 80% (w/w) Gelucire 44/14 and 5% (w/w) Ac-Di-Sol—100 mg drug/capsule
12. 10% (w/w) drug and 45% (w/w) Gelucire 44/14 and 45% (w/w) PEG-400-50 mg drug/capsule
13. 10% (w/w) drug and 85% (w/w) Gelucire 44/14 and 5% (w/w) Gelucire 50/13-50 mg drug/capsule
14. 10% (w/w) drug and 85% (w/w) Gelucire 44/14 and 5% (w/w) precirol—50 mg drug/capsule
15. 10% (w/w) drug and 88% (w/w) Gelucire 44/14 and 2% (w/w) campritol—50 mg drug/capsule
16. 10% (w/w) drug and 85% (w/w) Gelucire 44/14 and 5% (w/w) campritol—50 mg drug/capsule

### Example 3. Evaluation of the Anti-Inflammatory Activity of 25HC3S Administered Intradermally in an Imiquimod (IMQ)-Induced Psoriasis Model in Mice

### Materials and Methods

### Animals

The subjects for the study were 40 male Balb/C mice (18-22 g). Animals exhibiting no signs of clinical distress, disease or injury during a 72-hr quarantine period were accepted for the study and received routine animal care throughout. The backs of all mice were shaved for an area of about 1.5 cm×2 cm.

### Formulations

Two formulations of 25HC3 S, Formulation A and Formulation B, were used for the study.

Formulation A was a clear solution of 25HC3S sodium salt (30 mg/mL) in a solution vehicle (250 mg/mL hydroxypropyl betadex (beta cyclodextrin, 2-hydroxypropyl ether, a partially substituted poly(hydroxypropyl) ether of beta cyclodextrin) and 10 mM sodium phosphate buffer in sterile water). Vehicle was stored at 2-8° C. storage and placed at room temperature for 30 min. prior to mixing with powdered 25HC3S just prior to use. Dissolution of the 25HC3S in Vehicle A was rapid and appeared to be complete upon mixing. The concentration of 25HC3 S in solution was 30 mg/ml.

Formulation B was a milky suspension of 25HC3S sodium salt (25 mg/mL) in a suspension vehicle (30 mg/mL polyethylene glycol 3350, 3 mg/mL polysorbate 80, 7.5 mg/mL NaCl, and 10 mM sodium phosphate buffer in sterile water). The 25HC3 S was milled using a Fluid Energy Model 00 Jet-O-Mizer to approximately 5 microns average particle size (measured by a Malvern Mastersizer 2000 equipped with a Hydro 2000S dispersion cell). Vehicle was stored at 2-8° C. storage and placed at room temperature for 30 min. prior to mixing with powdered 25HC3S just prior to use. Because Formulation B is a suspension, the following mixing protocol was used: 3.0 mL of suspension vehicle was added to a vial containing pre-weighed powdered 25HC3S. The vial was shaken for 15 minutes on a flatbed shaker to create a uniformly white suspension, and then manually inverted 5-10 times, and shaken for 5 more minutes. In addition, immediately before administration, the vial was manually inverted 5-10 times to ensure uniformity of suspension.

### Administration of IMQ, Vehicle and 25HC3S

IMQ was applied topically once daily in the morning to the shaved back skin (50 mg) and the right ear (12.5 mg) of each mouse in order to induce psoriasis-like conditions.

The 25HC3S in vehicle or the vehicle alone were administered once on Days 0 and 1 and once on Days 3 and 4 by intradermal injection. Injections were done approximately 6 hours after the day's IMQ application. Intradermal injections (50 μL/injection/mouse) were given into the site of the back skin lesion.

### Monitoring and Measuring Parameters

Mice were monitored for signs of distress and daily photos of the back lesions were taken. Erythema, scaling, and thickness of the back skin was scored daily on a scale from 0 to 4 by an independent scorer (blind), where 0=none; 1=slight; 2=moderate; 3=marked; and 4=very marked. A cumulative score (erythema+scaling+thickening) was calculated as an indicator of the severity of the inflammation (on a scale of 0-12). Ear and back skin thickness was measured by electronic calipers as an indicator of edema.

### Termination (Day 6)

All mice in the study were anesthetized and exsanguinated. The blood was collected, processed to sera and stored at −80° C. for analytical use.

### Histopathology

The shaved back skin was collected from each animal at termination, weighed and cut into two halves (cut in half down the middle along the spine). One half was preserved in 10% neutral buffered formalin for histopathology. The other half of back skin was homogenized for measurement of cytokines TNFα and IL-17.

### Results

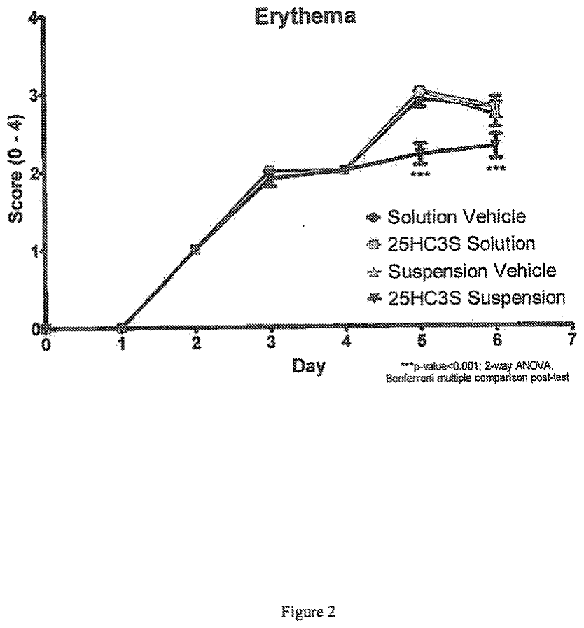

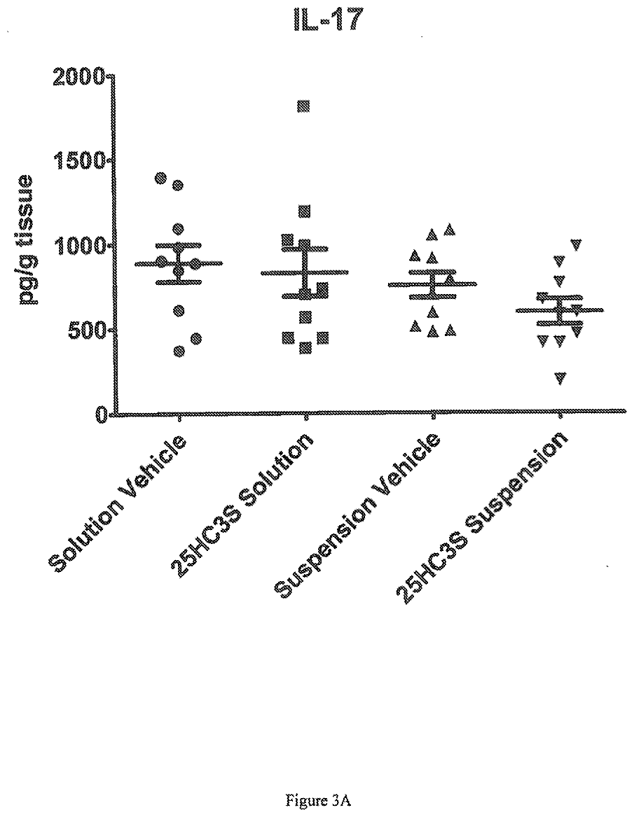

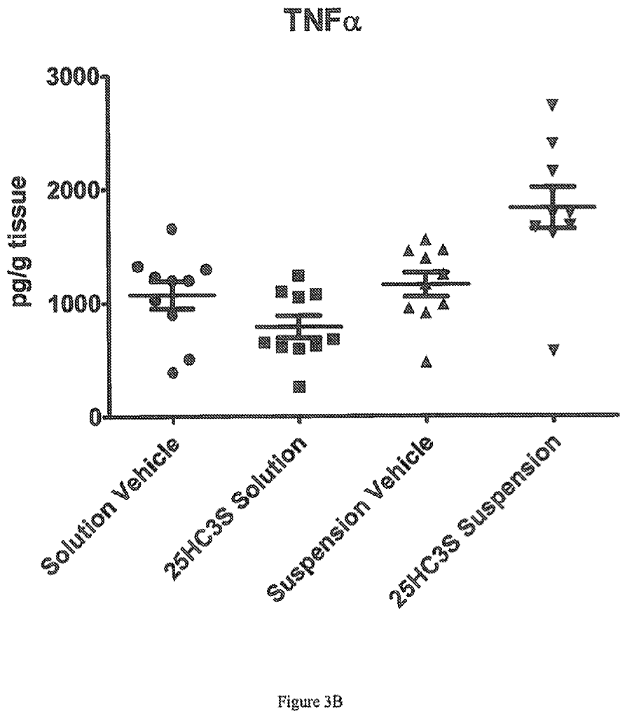

The results of this study are presented in FIGS. 2 and 3A and 3B. As can be seen in FIG. 2, erythema (redness) of the back skin was significantly reduced in mice treated with the Formulation B suspension. Erythema of the back skin was not significantly reduced in mice treated with the Formulation A, and erythema of the right ear was not significantly reduced in mice treated with Formulation A or B.

FIGS. 3A and 3B show IL-17 and TNFα protein levels, respectfully, in psoriatic skin/lesions as measured by ELISA assays. As can be seen, IL-17 trended lower in the Formulation B group compared to the respective vehicle group whereas no major differences were observed the Formulation A and its vehicle groups. In contrast, TNFα protein levels were modestly reduced in the skin tissue of Formulation A-treated mice compared to vehicle while increased in Formulation B-treated mice compared to its respective vehicle. While these results seem contradictory, one caveat of this study is that depending on where the tissue was collected (at the site of the intradermal injection which was contained to a small region of the lesion versus unexposed regions of the psoriatic lesion), protein levels may be dramatically variable within treatment groups. In all, we find that 25HC3S promotes reduction in erythema in a rodent model of psoriasis.

### Example 4. Preclinical Pharmacokinetic (PK) Injection Studies

Two PK injection studies have been performed using the 25HC3S suspension formulation containing PEG. Injection studies were conducted as follows: I. an acute (single dose) subcutaneous (SC) injection study in dogs and II. an acute (single dose) intramuscular (IM) or an acute SC injection study in rats.

### I. A Single SC Injection PK Study in Beagle Dogs

### Materials and Methods

Animals

The subjects for the study were 5 male Beagle dogs (4-7 years of age; 8-11 kg). Animals exhibiting no signs of clinical distress, disease or injury after the acclimatization period were accepted for the study and received routine animal care throughout. All animals were in healthy condition and admitted to the study.

### Formulation

A suspension formulation of 25HC3 S sodium salt was used for the study. The Vehicle was a solution of 3% (w/v) polyethylene glycol 3350, 0.3% (w/v) polysorbate 80, 0.7% (v/v) sodium chloride, 0.15% (w/v) L-methionine, 10 mM sodium phosphate buffer at pH 7.4 in water. 25HC3 S was mixed into the Vehicle solution to result in a drug concentration of 25 mg/mL. The mixture was shaken approximately 30 times to mix the 25HC3S powder and the vehicle together and subsequently sonicated at full power for approximately 30 minutes after which there was a milky white suspension. The formulated test article was used within 24 hours of constitution.

### 25HC3S Administration

Each dog received a single subcutaneous injection. The dose level of 25 mg/kg was administered in a dose volume of 1 mL/kg. Whole blood samples were collected via the jugular vein at pre-dose, 0.5, 1, 2, 4, 8, 12, 24, and 32 hours (h) post dose. Blood samples were placed into tubes containing K2EDTA. The blood was gently mixed to assure distribution of the anti-coagulant and the resulting plasma samples underwent analyses to quantify 25HC3S levels. During the in-life period, animals were observed for clinical signs within 4 hours post-dose on Day 1 and on Day 2. Assessments included, but were not limited to, evidence of pain on injection, assessment of activity, posture, respiration, emesis, seizure, hydration status, injection site assessment. There were no observable clinical signs.

### Results

A single SC dose of 25 mg/kg 25HC3S resulted in rapid absorption observed with a mean time to maximum plasma drug concentration at 23.2 h. Considerable variability was observed in maximum plasma concentration. The mean concentration at 32 h was 157.6 ng/mL.

### II. A Single SC or IM Injection PK Study in Rats

### Materials and Methods

### Animals

The subjects for the study were 15 male Sprague Dawley rats (8-11 weeks of age; 280-327 g at time of dosing). Animals exhibiting no signs of clinical distress, disease or injury after the acclimatization period were accepted for the study and received routine animal care throughout. All animals were in healthy condition and admitted to the study.

### Formulation

A suspension formulation of 25HC3 S was used for the study. The Vehicle was a solution of 3% (w/v) polyethylene glycol 3350, 0.3% (v/v) polysorbate 80, 0.7% (w/v) sodium chloride, 0.15% (w/v) L-methionine, 10 mM sodium phosphate buffer at pH 7.4 in water. 25HC3S was mixed into the Vehicle solution to result in drug concentrations of 25, 5 and 10 mg/mL. The mixture was shaken approximately 30 times to mix the 25HC3S powder and the vehicle together and subsequently sonicated at full power for approximately 30 minutes after which there was a milky white suspension. The formulated test article was used within 24 hours of constitution.

### 25HC3S Administration

Each rat received a single IM or SC injection (2 doses) (N=5/group). The dose level for the IM injection was 25 mg/kg was administered in a dose volume of 1 mL/kg. There were two dose groups for the SC injection route. The dose levels for the SC injections were 25 and 50 mg/kg with a dose volume of 5 mg/mL for both groups (drug concentrations were 5 and 10 mg/mL, respectively). Whole blood samples were collected via the jugular vein or the submandibular vein at pre-dose, 0.5, 1, 2, 4, 8, 12, 24, and 32 hours (h) post dose from each rat; however, the last blood collection may have been collected by terminal cardiac puncture with the animals deeply anesthetized by isoflurane. Blood samples were placed into tubes containing K2EDTA. The blood was gently mixed to assure distribution of the anti-coagulant and the resulting plasma samples underwent analyses to quantify 25HC3 S levels. During the in-life period, animals were observed for clinical signs. Assessments included, but were not limited to, assessment of activity, posture, respiration, emesis, seizure, hydration status, injection site assessment and overall body condition. There were no observable clinical signs.

### Conclusion

Both the IM and SC doses of 25 mg/kg resulted in similar plasma concentrations of 25HC3S. The two SC doses (25 and 50 mg/kg) did not exhibit proportional plasma dose concentrations. The IM group was observed to have a mean time to maximum plasma drug concentration at 10.4 (±2.2) hr while the two SC groups (25 and 50 mg/kd) were observed to reach maximum drug levels at 7.6 (±4.6) and 7.2 (±1.8) hrs. The mean maximum concentrations for the three groups were 101.9 (±17.1), 127.1 (±93.8) and 76 (±15.9) ng/mL and the mean concentrations at 32 h were 30 (±6.9), 35 (±10.3) and 34.2 (±13.8) ng/mL, respectively.

### Example 5. 25HC3S Shows Efficacy in an Accelerated Mouse Model of NASH—PART I

### Materials and Methods

### Animals

The subjects for the study were 30 C57BL/6J male mice. Mice were given a 200 ug streptozotocin (STZ) at 2 days after birth and fed high fat diet (HFD) starting at four weeks of age until the remainder of the study (9 weeks of age). This intervention early in their lives induces accelerated progression of non-alcoholic steatohepatitis (NASH) and has been highly characterized.

### Formulation

A suspension formulation of 25HC3S sodium salt and its respective vehicle was used for the study. The Vehicle was a solution of 0.5% (w/v) CMC and 0.05% (v/v) Tween-80 in water. Powdered 25HC3 S was constituted into the vehicle solution to result in drug concentrations of 5 and 10 mg/mL. The suspensions were homogenized for approximately 5 minutes being combined, with 10 second breaks every 30-40 seconds and swirled before dosing to maintain homogeneity. The formulated test article was prepared weekly and kept at room temperature.

### 25CH3S Administration

Mice were divided into treatment groups (N=10/group) and dosed daily by oral gavage with vehicle, 10 mg/kg or 50 mg/kg 25HC3S starting from Week 5 to Week 9 (28 days treatment).

### Results

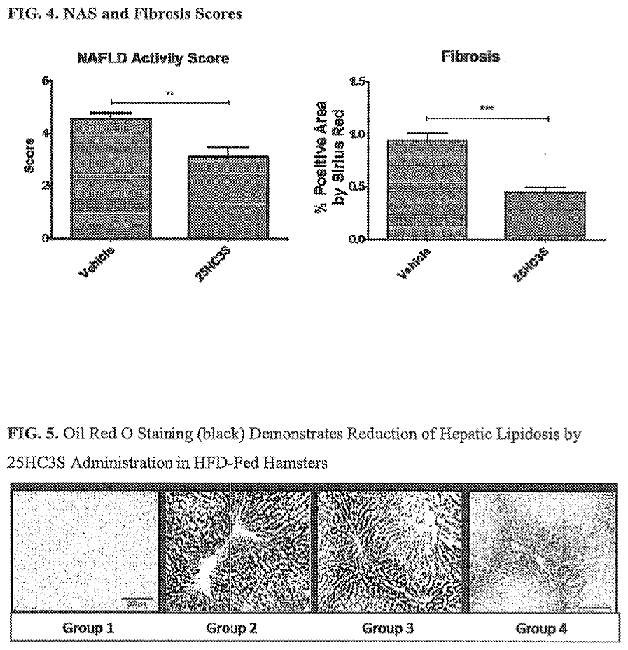

Histopathological examination of liver sections collected at the end of the study (Week 9) exhibited moderate to severe micro- and macrovesicular fat deposition, severe hepatocellular ballooning and inflammatory cell infiltration in vehicle-treated mice. 25HC3 S treatment displayed dose-dependent effects in the 50 mg/kg group showing marked improvement as reflected by a significant reduction in NAS (NAFLD activity score) compared to the vehicle group (FIG. 4; p=0.0088). No obvious changes were observed in H&E-stained sections between the vehicle group and the 25HC3S-10 mg/kg group (data not shown). Consistent with reduced NAS, the percent area of fibrosis (Sirius red-positive area) was also significantly decreased in the 50 mg/kg treatment group when compared to the vehicle group (FIG. 4; p=0.0061). There was no significant difference in the percent area of fibrosis between vehicle and 25HC3S-10 mg/kg treatment groups (data not shown).

In summary, a daily oral treatment of 25HC3 S (50 mg/kg) for four weeks significantly decreased NAS compared to vehicle at the time of sacrifice. 25HC3 S (50 mg/kg) also showed decreased fibrosis, as measured by Sirius red staining, compared to vehicle treatment. Together, these results suggest that 25HC3 S exhibited anti-NASH effects and may have the potential to slow the progression of fibrosis in NASH.

### Example 6. Non-GLP Pharmacokinetic and Pharmacodynamic Study of 25HC3 S in Golden Syrian Hamsters

### Materials and Methods

### Animals

The subjects for the study were 40 Golden Syrian male hamsters. Two cohorts were provided with either regular diet (RD) or high fat diet (HFD) for 10 weeks. 25HC3 S treatment was initiated at the start of Week 11. Group 1 remained on a regular diet while HFD-fed hamsters were randomly divided into three treatment groups (Groups 2-4; Table 20).

### Formulation

A suspension formulation of 25HC3 S sodium salt and its respective vehicle was used for the study. The Vehicle was a solution of 0.5% (w/v) CMC and 0.05% (v/v) Tween-80 in water. Powdered 25HC3 S was constituted into the vehicle solution to result in drug concentrations of 2.5 and 10 mg/mL. The suspensions were homogenized for approximately 5 minutes being combined, with 10 second breaks every 30-40 seconds and swirled before dosing to maintain homogeneity. The formulated test article was prepared weekly and kept at room temperature.

### 25HC3S Administration

Hamsters were treated with 25HC3 S by daily oral gavage for 6 weeks while maintained on RD or HFD. Each hamster received daily doses of 25HC3S or vehicle by oral gavage (N=10/group). There were two dose groups (Groups 3 and 4): 10 and 50 mg/kg, as specified in Table 20, with dose volumes of 4 and 5 mL/kg, respectively.

For pharmacokinetic (PK) analysis, blood was collected following the first 25HC3S dose. Whole blood samples were collected via the jugular vein at pre-dose, 0.5, 2, 4, 8, 12 hours (h) post dose from each hamster; however, the last blood collection may have been collected by terminal cardiac puncture with the animals deeply anesthetized by isoflurane. Blood samples were placed into tubes containing K2EDTA. The blood was gently mixed to assure distribution of the anti-coagulant and the resulting plasma samples underwent analyses to quantify 25HC3S levels.

For pharmacodynamic measures of efficacy, clinical chemistry parameters were measured by collection of fasting serum throughout the study to assess the effects of HFD and 25HC3S treatment compared to animals on a RD and given the vehicle control.

During the in-life period, animals were observed for clinical signs. Assessments included, but were not limited to, assessment of activity, posture, respiration, emesis, seizure, hydration status, injection site assessment and overall body condition. At the end of the in-life portion of the study (Week 16), all animals were sacrificed and livers collected for biochemical and histopathology analyses.

TABLE 20
25HC3S Administration (Weeks 11-16)

| Group | Dust | Treatment | Dose (mg/kg) |
|---|---|---|---|
| 1 | Regular | Vehicle | 0 |
| 2 | High fat | Vehicle | 0 |
| 3 | High fat | 25HC3S | 10 |
| 4 | High fat | 25HC3S | 50 |

### Results

Pharmacokinetics of 25HC3 S by oral administration was determined in HFD-fed hamsters after the first dose. Mean maximum plasma concentration of 25HC3 S was observed at 0.5 h for both doses with concentrations gradually declining until 12 h. The mean half-life was observed to be 3 hours. Increases in maximum plasma concentration and cumulative exposure (AUC) were not dose proportional following oral dosing. The normalized Cₘₐₓ for the 50 mg/kg dose was only half of the 10 mg/kg dose (32.2 ng/mL/mg); the dose-normalized AUC for the 50 mg/kg dose exhibited a similar decrease compared to the 10 mg/kg dose (195 ng*hr/mL/mg).

PO administration of 25HC3 S, daily for 6 weeks, did not result in any notable clinical signs. Although not statistically significant, 25HC3S treatment of HFD-fed hamsters produced a dose- and time-dependent reduction of serum cholesterol levels in the high dose (50 mg/kg) group (Group 4). 6 weeks of treatment (Week 16) resulted in a reduction in serum cholesterol levels (˜15-18%) in the high dose group. In contrast, serum triglyceride levels were trending higher in the treated groups (non-dose dependent and not statistically significant) compared to the vehicle group across the 6 weeks of treatment.

At the end of the study (Week 16), serum levels of HDL, LDL and ALT, AST and ALK were measured. As expected, HFD-fed hamsters had significantly elevated HDL and LDL cholesterol levels compared to RD-fed hamsters. Consistent with total serum cholesterol levels, 6 weeks of 25HC3 S treatment reduced both HDL and LDL cholesterol in a dose-dependent fashion in HFD-fed hamsters. Compared to RG, HFD-fed hamsters had higher ALT and AST levels, indicating hepatic injury. However, 25HC3 S treatment reduced both ALT and AST levels compared to vehicle. In this study, ALK levels were reduced in all HFD-fed hamsters (compared to RD fed hamsters) regardless of drug treatment.

25HC3 S treatment had no statistically significant effect on HFD-related liver weight gain. However, gross necropsy indicated a 22% incidence of “normal-appearing” livers (per pathologist assessment) in Group 4 animals compared to a 0% incidence of “normal-appearing” livers in the vehicle-treated group on HFD (data not shown).

Liver tissues were quantified for total cholesterol, free cholesterol, triglyceride and free fatty acid (FFA) levels in RD- and HFD-fed hamsters. Compared to RD-fed controls, HFD-fed hamsters had significant accumulation of hepatic total cholesterol, free cholesterol and triglycerides (Table 21). Free fatty acids levels were not increased with HFD. Treatment with 25HC3 S for 6 weeks significantly reduced hepatic cholesterol levels at the higher 25HC3 S dosage (Group 4 with no effect seen in hamsters given 10 mg/kg. Reduced hepatic triglyceride levels were also observed with increasing 25HC3 S dosage, although the results did not reach statistical significance (Table 21).

TABLE 21
Quantified Hepatic Lipids in HFD-fed Hamsters

| | | | | Total | |
|---|---|---|---|---|---|
| | | | Triglyceride | Cholesterol | Free Fatty |
| | | 25HC3S | (μg/mg | (μg/mg | Acids (Meg/ |
| Group | Diet | (mg/kg) | protein) | protein) | mg protein) |
| 1 | Regular | 0 | 35.9 (±8.4) | 20.4 (±1.7) | 9.0 (±2.8) |
| 2 | High fat | 0 | 60.7 (±20.9)* | 166.7 (±67)* | 8.0 (±0.9) |
| 3 | High fat | 10 | 66.1 (±23.3) | 155.5 (±37.9)** | 7.3 (±0.6) |
| 4 | High fat | 50 | 46.4 (±15.6) | 75.2 (±45.7)** | 6.6 (±1.4) |

*p < 0.05 compared to Group 1
**p < 0.05 compared to Group 2

Histopathology was performed on livers collected at the end of the study. Standard H&E and Oil Red 0 staining revealed hepatic microvesicular lipidosis (distended cytoplasm with small, fine vacuoles positive for Oil Red 0 staining) present in all HFD-fed groups, but not in the RD group. In addition, mild multifocal non-suppurative inflammation and some glycogen accumulation were also present in the HFD-fed hamster livers. In a dose-dependent fashion, considerably less microvesicular changes, reduced Oil Red 0 staining, and milder inflammation was observed with 25HC3S treatment compared to the HFD-fed control animals (Group 2). See FIG. 5.

### Example 7. Non-GLP Pharmacodynamic Study of 25HC3 S in the Acetaminophen (APAP)—Induced Model of Acute Liver Failure

### Materials AND METHODS

### Animals

The subjects for the study were 52 C57BL/6J male mice (12 weeks of age; 27.4-40 g). Animals exhibiting no signs of clinical distress, disease or injury after the acclimatization period were accepted for the study and received routine animal care throughout. All animals were in healthy condition and admitted to the study.

### Formulation

A suspension formulation of 25HC3 S sodium salt and its respective vehicle was used for the study. The Vehicle was a solution of 0.5% (w/v) CMC and 0.05% (v/v) Tween-80 in water. Powdered 25HC3 S was constituted into the vehicle solution to result in a drug concentration of 3 mg/mL. The suspensions were homogenized at 20,000 rotations per minute (rpm) for approximately 5 minutes after being combined, with 10 second breaks every 30-40 seconds and swirled before dosing to maintain homogeneity. The formulated test article was prepared weekly and kept at room temperature.

APAP and 25HC3S Administration

Two groups of mice (N=14/group) were challenged with 300 mg/kg APAP by oral gavage. The two groups were treated with one dose of vehicle or 25HC3 S (25 mg/kg) by oral gavage at a dose volume of 8.33 mL/kg one hour post-APAP challenge. Half of the mice in each group (N=6-7/group) were given a second dose of vehicle or 25HC3 S (25 mg/kg) at 24 hour post-APAP delivery in addition to the first dose at 1 hr. Cohort A mice (single dose) were sacrificed 24 hrs post APAP-challenge and Cohort B mice (two doses) were sacrificed at 48 hours post APAP-challenge. A parallel set of untreated age-matched mice (no APAP and vehicle administered by oral gavage) were also sacrificed at both time points to compare baseline measurements (N=6/time point; 12 in total). Overnight fasted blood was collected by cardiac puncture at the time of euthanasia. Blood samples were allowed to clot and the serum was harvested to measure serum ALT, AST, ALK, LDH, BUN and glucose.

### Results

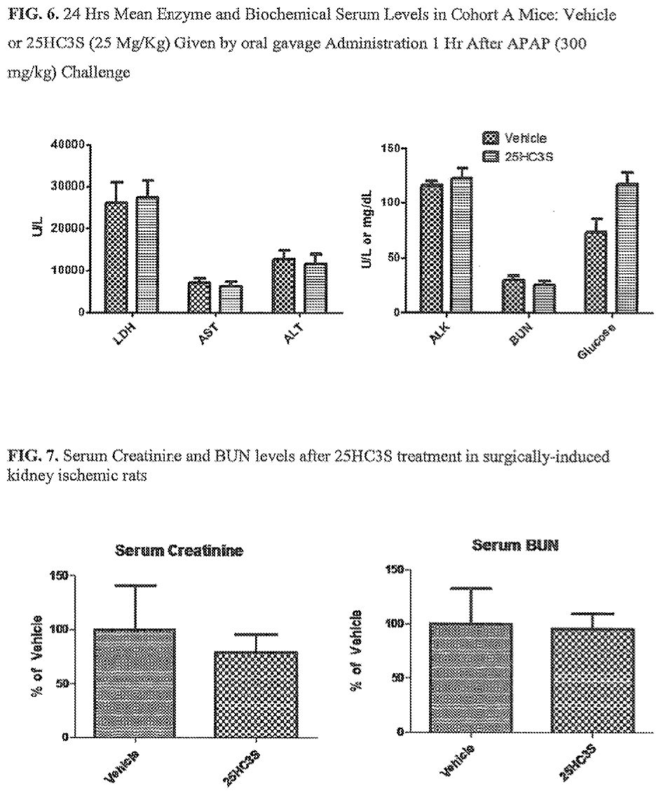

In this study, APAP resulted in a large and similar increase in LDH, ALT, and AST levels in Cohort A mice (single dose; 24 hrs). BUN levels were also slightly elevated whereas ALK and glucose levels were minimally changed. At 48 hrs, a similar pattern of induction was observed in Cohort B mice, although measured values were substantially lower, indicating strong self-recovery under these experimental conditions. Treatment with 25HC3 S (25 mg/kg) demonstrated no effect on serum chemistry parameters measured in either cohorts compared to their respective vehicle controls (FIG. 6). In conclusion, oral administration of 25HC3 S does not lower serum biochemical markers after a semi-APAP-induced liver failure.

### Example 8. Effect of 25HC3 S in the Prevention and Treatment of Renal Ischemia/Reperfusion Injury in Rats

### Materials and Methods

### Animals

The subjects for the study were 18 adult male Lewis rats (9-11 weeks of age; 225-250 g). Animals exhibiting no signs of clinical distress, disease or injury after the acclimatization period were accepted for the study and received routine animal care throughout. All animals were in healthy condition and admitted to the study.

### Formulation

A suspension formulation of 25HC3 S sodium salt and its respective vehicle was used for the study. The Vehicle was a solution of 0.5% (w/v) CMC and 0.05% (v/v) Tween-80 in water. Powdered 25HC3 S was constituted into the vehicle solution to result in a drug concentration of 20 mg/mL. The suspensions were homogenized at 20,000 rotations per minute (rpm) for approximately 5 minutes after being combined, with 10 second breaks every 30-40 seconds and swirled before dosing to maintain homogeneity. The formulated test article was prepared weekly and kept at room temperature.

### Renal Ischemia Induction & 25HC3S Administration

All rats were anesthetized with intraperitoneal injection of pentobarbital (40 mg/kg). Ischemia of the left kidney was achieved by transient occlusion of the left renal artery and vein, and ureter for 50 min with a vascular micro-clip. The skin was temporarily closed during the ischemia period and the rats were put on a heating pad maintained at a temperature of 37° C. At reperfusion, the right kidney was removed before permanently closing the abdomen with 4-0 silk suture Animals were treated with vehicle (N=6) or 25HC3S (N=12) daily for 4 days, starting on the day before the surgery, (pre-treatment, Day −1) and for 2 days after the surgery. Vehicle or 50 mg/kg 25HC3 S suspension was given by oral gavage at a dose volume of 5 mL/kg. Serum creatinine (sCr) levels and BUN levels were examined on Day −2 (baseline), Day 3, and/or Day 7 after the surgery.

### Results

Daily 50 mg/kg 25HC3S treatment for 4 days by oral gavage reduced sCr and BUN levels by ˜20% and 5% on Day 3 as compared to the vehicle group, although the differences did not reach statistical significance (FIG. 7). However, the data suggests 25HC3S may ameliorate acute kidney injury in this rat model.

### Example 9. Oral 25HC3S Capsule Formulations

Three capsule dosage formulations of 25HC3 S were used for the study. The summary of the different capsule formulations that were tested are described in Table 22.

TABLE 22
Capsule Dosage Formulations Information

| Capsule | | | |
|---|---|---|---|
| Formulation | A | B | C |
| 2511C3S dose | 50 mg | 50 mg | 50 mg |
| Inactive ingredients | Hypromellose | Hypromellose (HPMC) | Hypromellose (HPMC) |
| | (HPMC) capsule, size 0 | capsule, size 0 | capsule, size 0 |
| | Gelucire 48/16 | Gelucire 44/14 (lauroyl | Gelucire 44/14 (lauroyl |
| | (polyoxyl stearate) | polyoxylglycerides) | polyoxylglycerides) |
| | | Precirol ATO5 (glyceryl | PEG-400 (polyethylene |
| | | distearate) | glycol 400) |

### Formulation Preparation

Three bulk formulations were prepared in respective 500 mL I-Chem jars at 160 grams per batch as shown in Table 23. The formulation jars were immersed in water bath maintained at 60-65° C. throughout the process. Gelucire 48/16 or Gelucire 44/14 was heated in a 60° C. oven until melted. The Gelucire was manually mixed with a spatula prior to dispensing.

For Formulation A, powdered 25HC3 S was slowly added into the melted Gelucire 48/16 and mixed with a spatula until visually fully mixed. The formulation was further mixed under an overhead mixer at 500-1000 rpm for 20 minutes.

For Formulations B and C, Precirol ATOS or Pluriol E 400 (PEG-400) was added into melted Gelucire 44/14 and mixed under overhead mixer at 300-500 rpm for 10-15 minutes. Powdered 25HC3S was then slowly added and mixed with a spatula until visually fully mixed. The formulation was further mixed under an overhead mixer at 500-1000 rpm for an additional 20 minutes.

The bulk formulations were manually filled into size 0 HPMC capsules with 500 mg of targeted capsule fill weight to achieve 50 mg dose strength per capsule.

TABLE 23
Formulation Composition (%, w/w)

| Formulation | | Gelucire | Gelucire | Precirol | Pluriol E |
|---|---|---|---|---|---|
| ID | 25HC3S | 48/16 | 44/14 | ATO 5 | 400 |
| A | 10 | 90 | 0 | 0 | 0 |
| B | 10 | 0 | 88 | 2 | 0 |
| C | 10 | 0 | 60 | 0 | 30 |

### Dissolution Testing

The release rate of 25HC3S was determined using a USP Apparatus 2 dissolution tester. Three capsules from each formulation were tested. Dissolution medium containing 1000 mL of 0.5% Triton X-100 in 0.1N HCl was maintained at 37° C. with 75 rpm paddle speed over the course of the 2-hour dissolution test. The standard sampling time points were 0.25, 0.5, 0.75, 1, and 2 hours. A 1 mL sample was taken at each time point and assayed using HPLC.

### Results

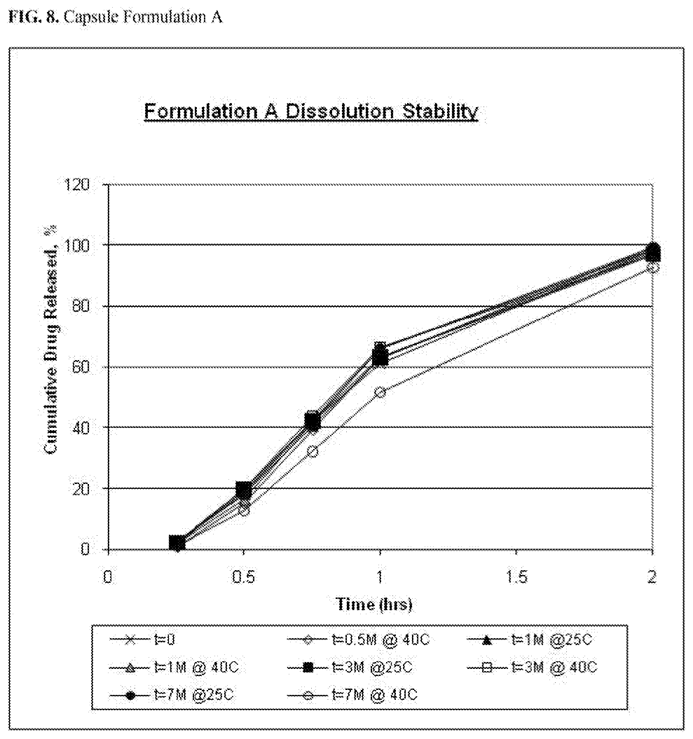

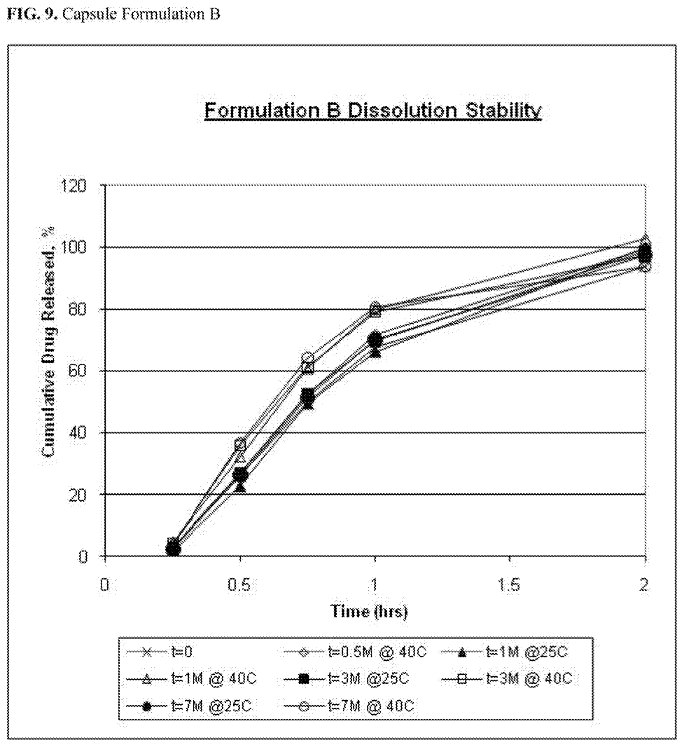

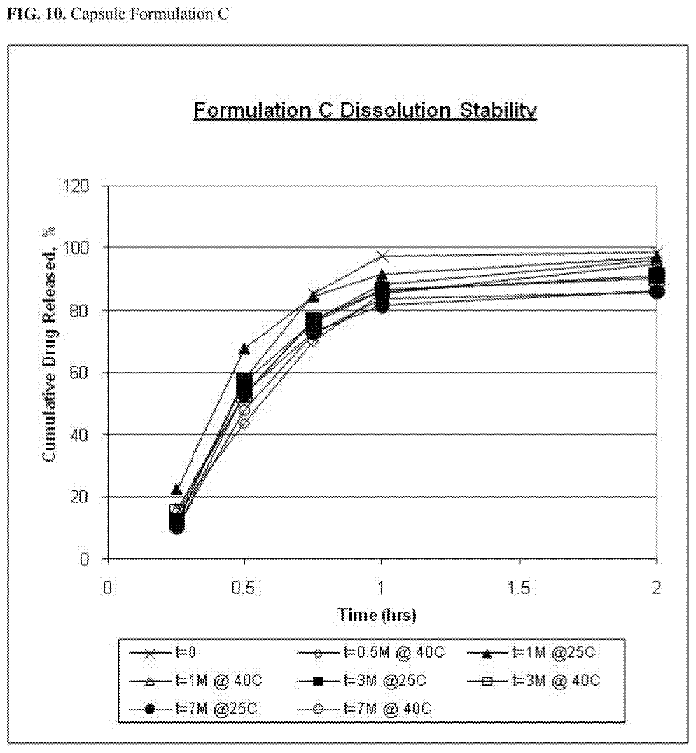

The results from the dissolution experiments for capsule formulations A-C are provided in FIGS. 8-10, respectively. As shown in FIGS. 8-10, each of the capsule formulations was tested at t=0; t=1, 3, and 7 months after storage at 25° C.; and t=0.5, 1, 3, and 7 months after storage at 40° C.

### Example 10. Non-GLP Pharmacokinetic (PK) Evaluation of 25HC3 S Oral Capsules in Beagle Dogs

### Materials and Methods

### Animals

The subjects for the study were 5 male Beagle dogs (4-7 years of age; 8-11 kg). Animals exhibiting no signs of clinical distress, disease or injury after the acclimatization period were accepted for the study and received routine animal care throughout. All animals were in healthy condition and admitted to the study.

### Formulation

Capsule formulations A-C, as described in above Example 9, were tested. An oral suspension formulation of 25HC3 S was also used for the study as a comparator relative to capsule formulations A-C.

Oral suspension preparation: The Vehicle was a solution of 0.5% CMC and 0.05% Tween-80 in water. Powdered 25HC3S was constituted into the vehicle solution to result in a drug concentration 10 mg/mL. The suspension was homogenized for approximately 5 minutes being combined, with 10 second breaks every 30-40 seconds and swirled before dosing to maintain homogeneity. The suspension was placed on a stir plate for at least 10 minutes prior to dosing and was left gently stirring throughout drug administration. All formulations were stored at room temperature.

### 25HC3S Administration

There were 3 different solid dosage forms and 1 oral suspension formulation. Each dog received a single oral dose of each of the 4 different 25HC3 S formulations with washout periods of 3-4 days between each administration of 25HC3 S. For the suspension, the dose level of 50 mg/kg was administered in a dose volume of 5 mL/kg, after which, the dog was flushed with 5 mL of water. Each dog was also administered a single 50 mg 25HC3S capsule for oral consumption for each of the 3 solid dosage forms and also flushed with 5 mL of water. Whole blood samples were collected via the jugular vein at pre-dose, 1, 2, 4, 8 and 24 hours (h) post dose. Blood samples were placed into tubes containing K2EDTA. The blood was gently mixed to assure distribution of the anti-coagulant and the resulting plasma samples underwent analyses to quantify 25HC3S levels. During the in-life period, animals were observed for clinical signs throughout the entirety of the study (14 days). Assessments included, but were not limited to, evidence of pain on injection, assessment of activity, posture, respiration, emesis, seizure, hydration status, injection site assessment.

### Results

A single oral dose of 50 mg/kg 25HC3S in Beagle dogs resulted in rapid absorption observed with a mean time to maximum plasma drug concentration at 1.5-3.5 h, across all formulations. Maximum plasma concentrations ranged from 173-304 ng/mL, with Capsule A exhibiting the lowest Cₘₐₓ and Capsule B exhibiting the highest. Half-lives for all formulations were similar (t_{1/2}=0.91-0.94h). Capsule B exhibited the highest level of systemic exposure as reflected by AUCₗₐₛₜ (807±156 ng*hr/mL) whereas Capsule A exhibited the lowest (552±153 ng*hr/mL). See Table 24.

TABLE 24
Mean Pharmacokinetic Parameters (SD)

| | | | | AUCₗₐₛₜ |
|---|---|---|---|---|
| Formulation | Tₘₐₓ (h) | Cₘₐₓ (ng/mL) | T_{1/2} (h) | (ng * hr/mL) |
| Suspension | 2.2 (1.1) | 200 (80) | 0.92 (0.13) | 589 (152) |
| Capsule A | 3.4 (1.3) | 173 (33) | 0.94 (0.20) | 552 (153) |
| Capsule B | 2.0 (1.2) | 304 (50) | 0.91 (0.13) | 807 (156) |
| Capsule C | 1.4 (0.6) | 261 (110) | 0.94 (0.25) | 763 (205) |

### Example 11. Capsule Formulation Study

### Objective

1) To better understand the effect of drug loading and each excipient on in vitro drug release profiles.

2) To develop some formulations having faster dissolution than the capsule formulation B described in above Example 9 and used in above Example 10.

### Background

A four factor definitive screening design was performed for 25HC3 S capsule formulation development and the formulation compositions shown in Table 25 and Table 26. The four variables evaluated included drug loading and three excipients (Gelucire 50/13, Labrasol, and Plurol CC497). Gelucire 44/14 served as a base excipient the amount of which was calculated by subtracting the total amount in percent of drug substance and three excipients from 100%.

TABLE 25
Four Variables and Ranges Used in Design

| Fill Weight = 500 mg | Variables | −1 | 0 | 1 |
|---|---|---|---|---|
| A | Drug Loading | 5% | 7.5% | 10% |
| B | Gelucire 50/13 | 5% | 15% | 25% |
| C | Labrasol | 0% | 5% | 10% |
| D | Plurol CC497 | 0% | 5% | 10% |

TABLE 26
Definitive Screening Design

| Definitive Screening 4 Factors | A | B | C | D |
|---|---|---|---|---|
| 1 | 1 | −1 | 1 | 0 |
| 2 | 0 | 0 | 0 | 0 |
| 3 | 0 | 1 | 1 | 1 |
| 4 | −1 | −1 | 0 | 1 |
| 5 | 1 | 0 | −1 | 1 |
| 6 | −1 | 0 | 1 | −1 |
| 7 | 0 | −1 | −1 | −1 |
| 8 | 1 | 1 | 0 | −1 |
| 9 | −1 | 1 | −1 | 0 |

### Formulation Preparation

Each formulation was prepared in a 125 mL I-Chem jar at 30 grams per batch as shown in Table 27. Each formulation jar was immersed in a water bath maintained at 50-55° C. throughout the process. Gelucire 44/14 was heated in a 60° C. oven until melted. The Gelucire was manually mixed with a spatula prior to dispensing. The melted Gelucire 44/14 was weighed out and added into each jar. Then individual excipients were weighed out and added into the melted Gelucire 44/14 with 5 minutes of overhead mixing at 300 rpm after each addition. Powdered 25HC3 S was slowly added into the mixture and mixed with a spatula until visually fully mixed. The formulation was further mixed under an overhead mixer at 500 rpm for 10 minutes. The bulk formulation was homogenized with setting “1” for 1 minute and overhead mixing at 500 rpm for 5 minutes. The final formulations were manually filled into HPMC capsules with 500 mg of targeted capsule fill weight except Formulation 11 with 333 mg and Formulation 12 with 400 mg.

TABLE 27
Formulation Composition (%, w/w)

| | | | | | Plurol | Drug | Cap- |
|---|---|---|---|---|---|---|---|
| Formu- | Drug | Gelucire | Gelucire | | CC | Dose | sule |
| lation | Loading | 44/14 | 50/13 | Labrasol | 497 | (mg) | Size |
| 1 | 10 | 70 | 5 | 10 | 5 | 50 | 0 |
| 2 | 7.5 | 67.5 | 15 | 5 | 5 | 37.5 | 0 |
| 3 | 7.5 | 47.5 | 25 | 10 | 10 | 37.5 | 0 |
| 4 | 5 | 75 | 5 | 5 | 10 | 25 | 0 |
| 5 | 10 | 65 | 15 | 0 | 10 | 50 | 0 |
| 6 | 5 | 70 | 15 | 10 | 0 | 25 | 0 |
| 7 | 7.5 | 87.5 | 5 | 0 | 0 | 37.5 | 0 |
| 8 | 10 | 60 | 25 | 5 | 0 | 50 | 0 |
| 9 | 5 | 65 | 25 | 0 | 5 | 25 | 0 |
| 10 | 5 | 90 | 5 | 0 | 0 | 25 | 0 |
| 11 | 7.5 | 82.5 | 5 | 5 | 0 | 25 | 1 |
| 12 | 6.25 | 88.75 | 5 | 0 | 0 | 25 | 1 |

Formulation 1-9: DOE runs
Formulations 10-12: Prediction runs

### Dissolution Testing

The release rate of 25HC3S was determined using a USP Apparatus 2 dissolution tester (n=4 replicates). Dissolution medium containing 1000 mL of 0.5% Triton X-100 in 0.1N HCl was maintained at 37° C. with 75 rpm paddle speed over the course of the 4-hour dissolution test. The standard sampling time points were 0.25, 0.5, 0.75, 1, 2, and 4 hours. A 1 mL sample was taken at each time point and assayed using HPLC.

### Results

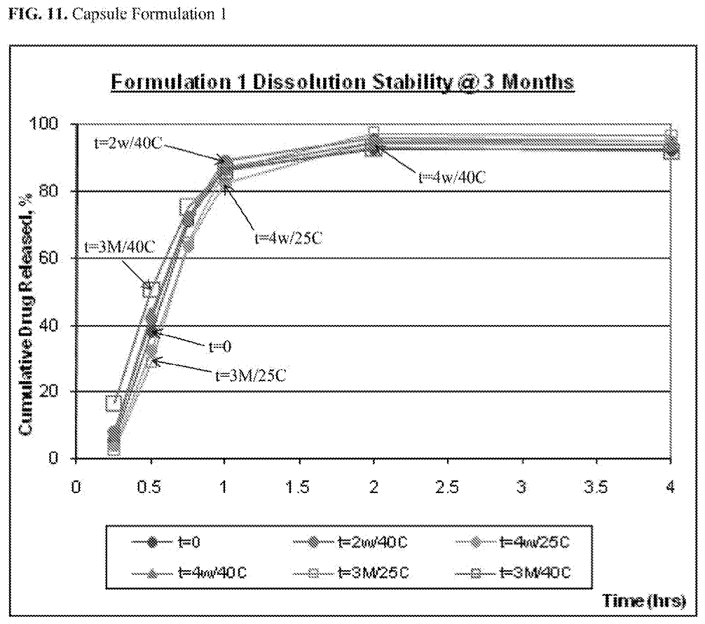

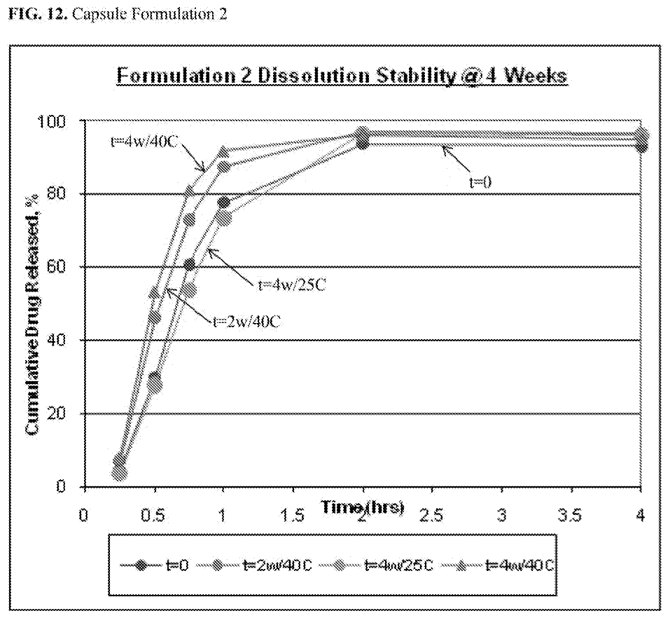

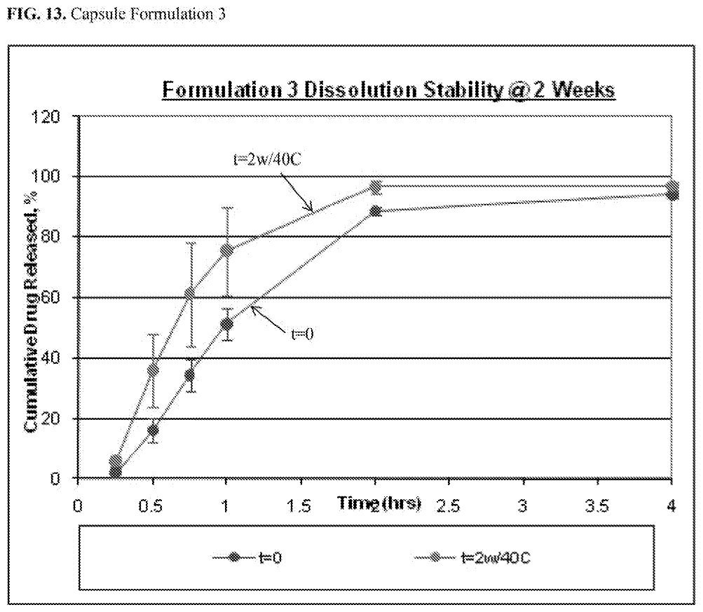

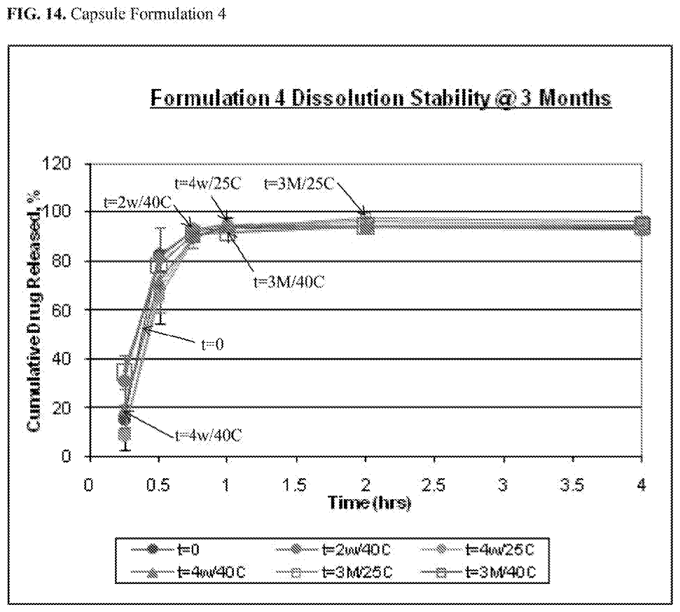

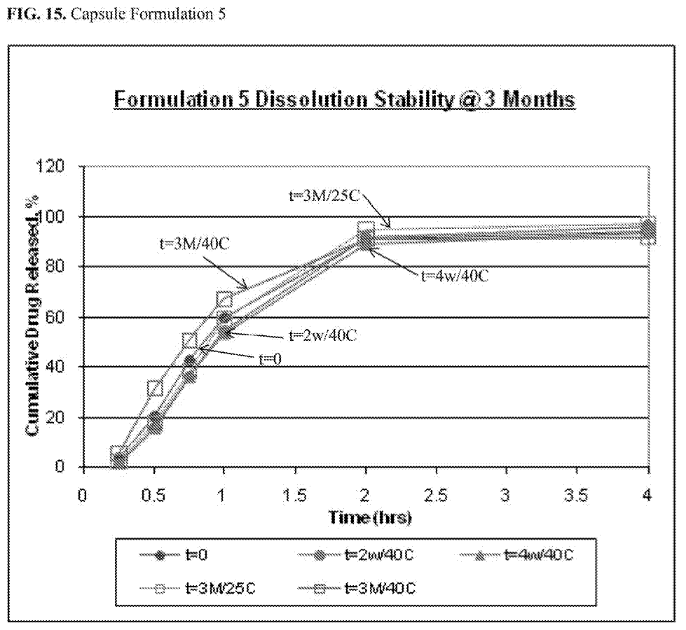

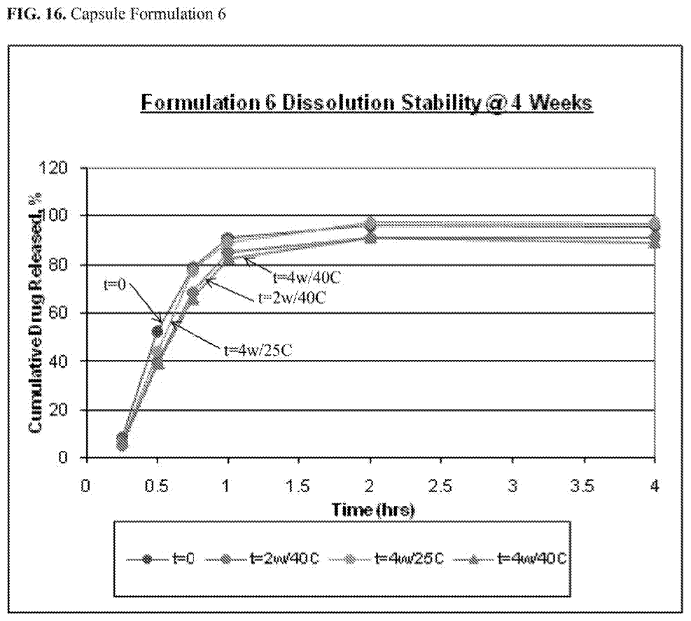

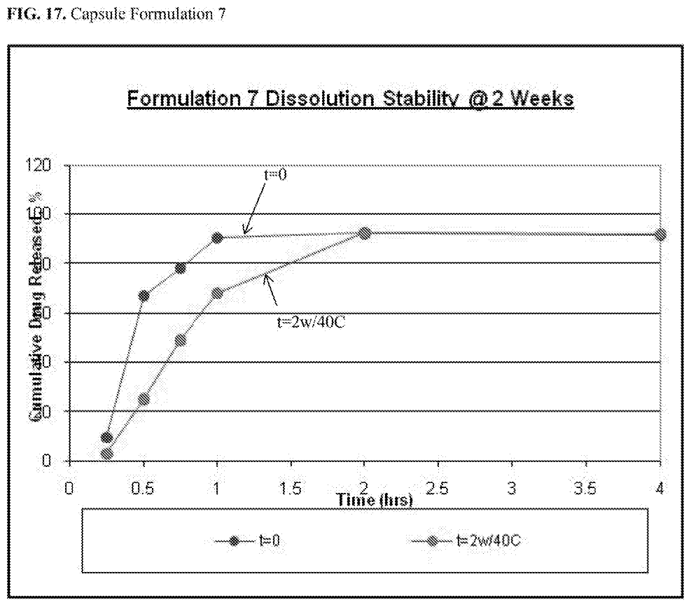

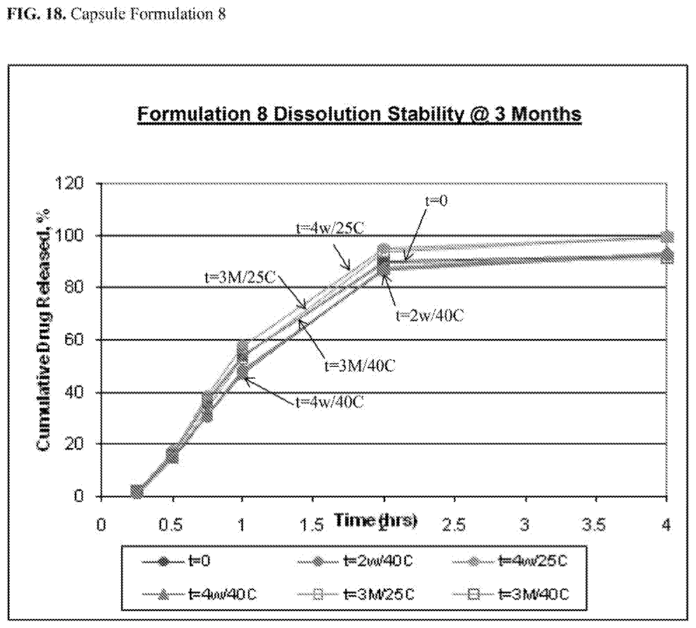

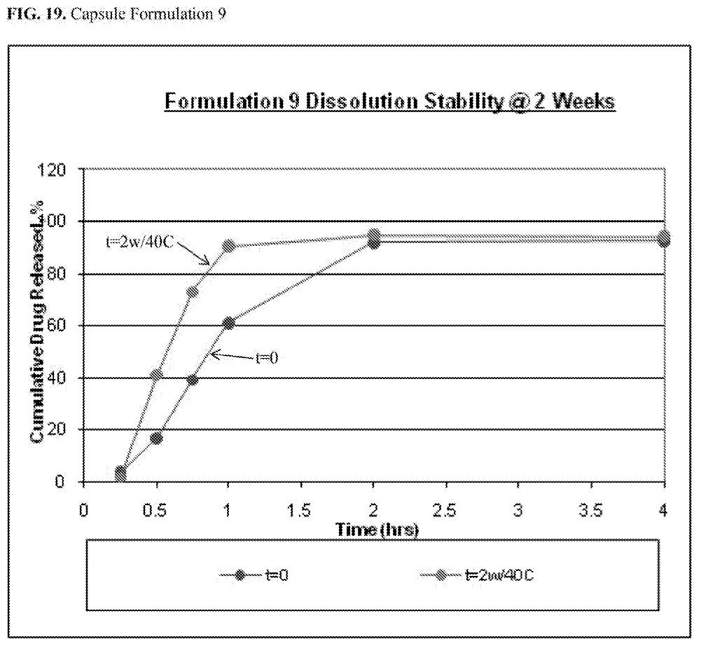

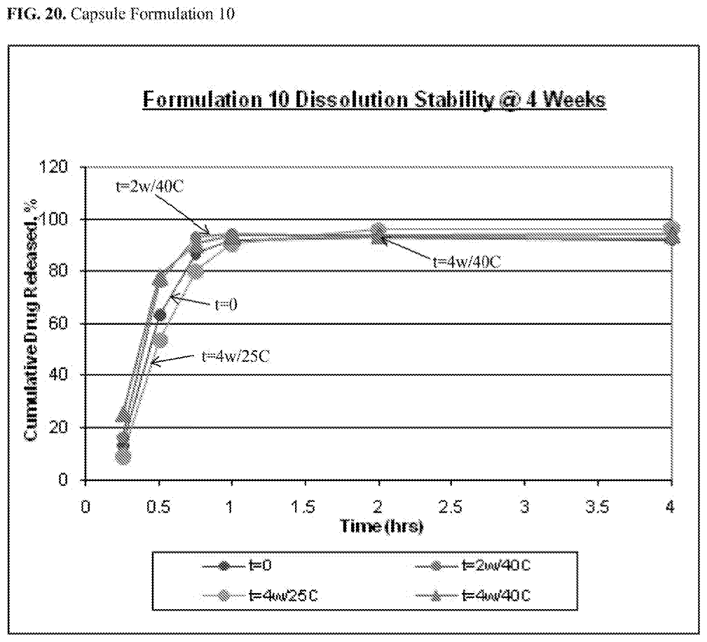

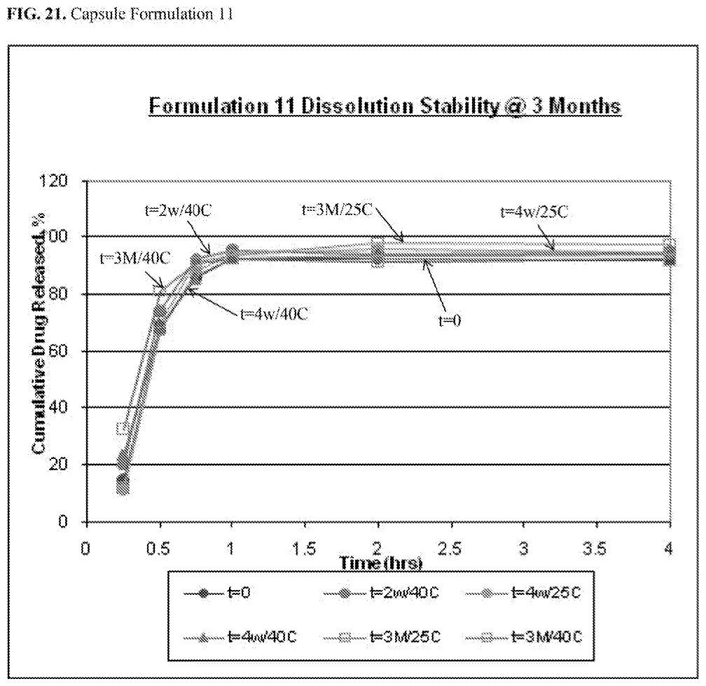

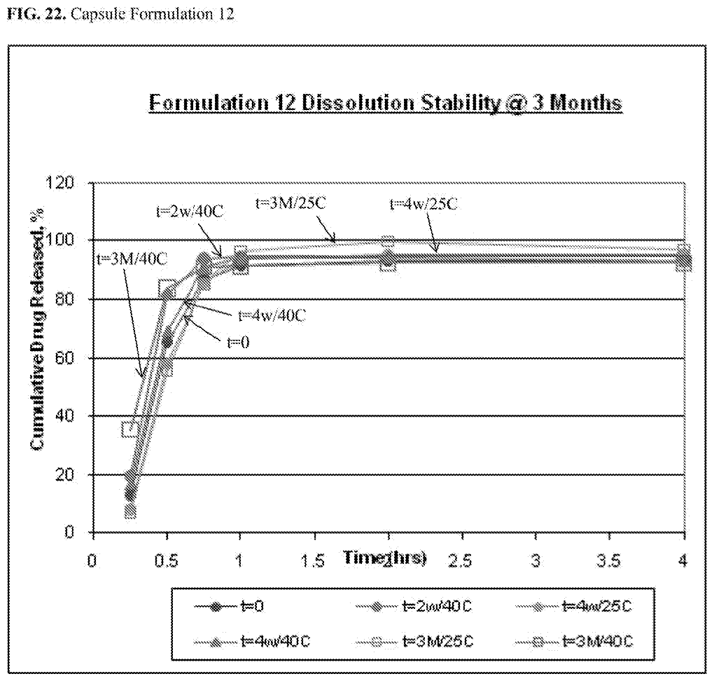

The results from the dissolution experiments for capsule formulations 1-12 are provided in FIGS. 11-22, respectively. As shown in FIGS. 11-22, the capsule formulations were tested at t=0 and after storage at different temperatures for different times.

### Example 12. 25HC3S Shows Efficacy in an Accelerated Mouse Model of NASH—PART II

### Materials and Methods

### Animals

The subjects for the study were 36 C57BL/6J male mice. Mice were given 200 μg streptozotocin (STZ) at 2 days after birth and fed high fat diet (HFD) starting at four weeks of age until the remainder of the study (13 weeks of age). This intervention early in their lives induces accelerated progression of non-alcoholic steatohepatitis (NASH) and has been thoroughly characterized.

### Formulation

A suspension formulation of 25HC3 S sodium salt and its respective Vehicle was used for the study. The Vehicle was a solution of 0.5% (w/v) CMC and 0.05% (v/v) Tween-80 in water. Powdered 25HC3 S was constituted into the Vehicle solution to result in drug concentration of 10 mg/mL. The suspensions were homogenized for approximately 5 minutes (with 10 second breaks every 30-40 seconds) and swirled before dosing to maintain homogeneity. The formulated test article was prepared weekly and kept at room temperature.

### 25HC3S Administration

Mice were divided into treatment groups (N=10/group) and dosed daily by oral gavage with water (control), Vehicle or 50 mg/kg 25HC3 S starting from Week 9 to Week 13 (28 days treatment).

### Results

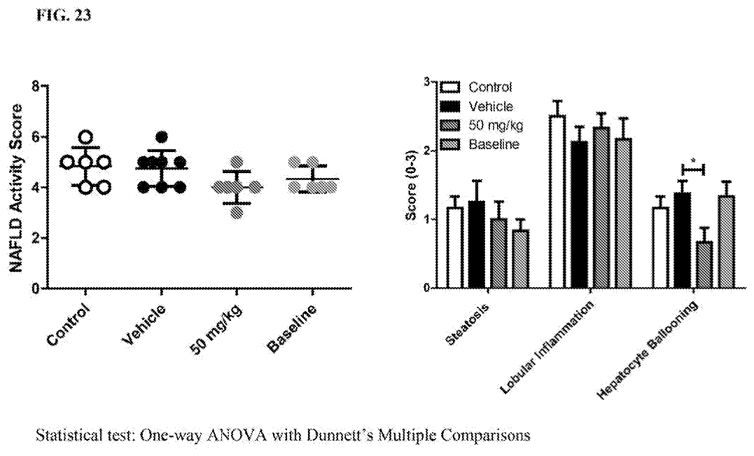

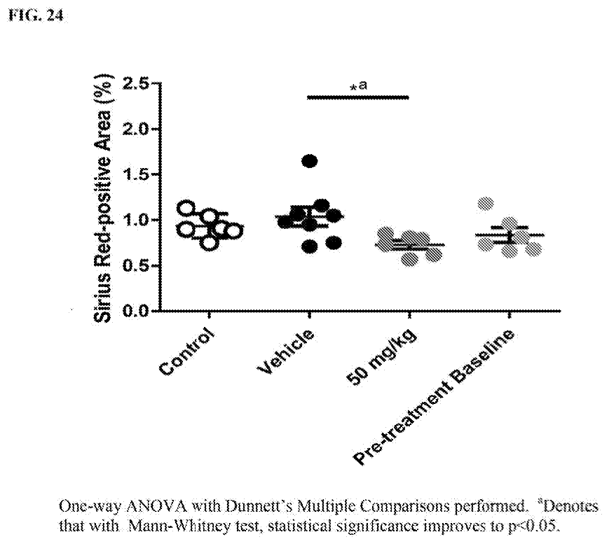

Histopathological examination of liver sections collected at the end of the study (Week 13) exhibited moderate to severe micro- and macrovesicular fat deposition, severe hepatocellular ballooning and inflammatory cell infiltration in water- and Vehicle-treated mice. 25HC3 S treatment displayed improvement as reflected by a significant reduction in hepatocyte ballooning (p<0.05), which resulted in a trend of reduction in NAS (NAFLD activity score) compared to the Vehicle group (FIG. 23) (For FIG. 23, right panel, treatment conditions for each group from left to right are as follows: control, vehicle, 50 mg/kg 25HC3S, and baseline). Consistent with reduced NAS, the percent area of fibrosis (Sirius red-positive area) was also significantly decreased with 25HC3 S treatment compared to the Vehicle group (FIG. 24; p<0.05). The extent of fibrosis also trended lower in the 25HC3S-treated group as compared to the baseline Week 9 mice (N=6/group) that were sacrificed at Week 9, suggesting that reversal of fibrosis by 25HC3S may also occur (FIG. 24).

In summary, daily oral treatment of 25HC3 S (50 mg/kg) for four weeks significantly decreased hepatocyte ballooning, a component of NAS, compared to Vehicle at the time of sacrifice. 25HC3 S also resulted in significantly decreased presence of fibrosis, as measured by Sirius red staining, compared to Vehicle treatment and reduced fibrosis compared to Week 9 baseline STAM mice. Together, these results suggest that 25HC3 S has antifibrotic effects and has the potential to slow the progression of fibrosis in NASH.

### Example 13. Efficacy of 25HC3 S in a Rodent Model of Cholestasis and Pharmacological Intervention of 25HC3 S in a Rodent Model of Cholestasis: Bile Duct Ligated (BDL) Rats

### Materials and Methods

### Animals

The subjects for the study were CD1 male rats (8 weeks of age, 200-225 g). Rats underwent BDL surgery, where the extrahepatic biliary tract was tightly ligated twice with sutures, then cut between the two ligations. A sham group (N=5/group) was also included in a subset of the studies described.

### Formulation

A suspension formulation of 25HC3 S sodium salt and its respective Vehicle was used for the study. The Vehicle was a solution of 0.5% (w/v) CMC and 0.05% (v/v) Tween-80 in water. Powdered 25HC3 S was constituted into the Vehicle solution to result in drug concentrations of 0.833 to 5 mg/mL. The suspensions were homogenized for approximately 5 minutes being combined, with 10 second breaks every 30-40 seconds and swirled before dosing to maintain homogeneity. The formulated test article was prepared weekly and kept at room temperature.

### 25CH3S Administration

Mice were divided into treatment groups (N=8-10/group) and dosed daily or every 3 days for 9 days by oral gavage. 5, 10, 30 or 60 mg/kg 25HC3S or Vehicle was given starting one day (Day 1) after BDL surgery. On Day 10, serum was collected after an overnight fast and measured for serum biochemistry.

### Results

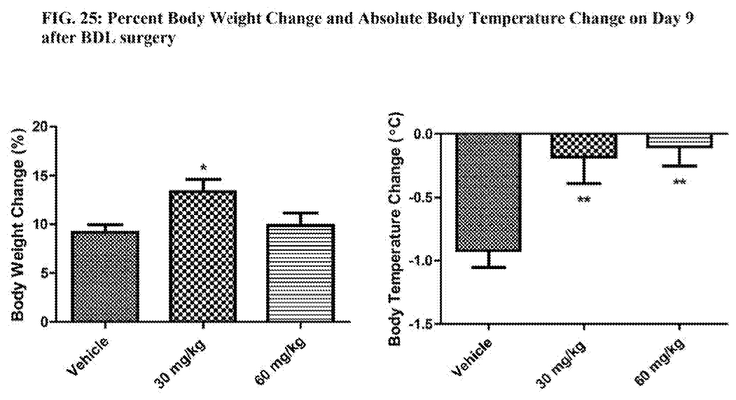

In a pilot study, daily dosing at 30 or 60 mg/kg 25HC3S (N=10/group) demonstrated a significant effect on body temperature compared to Vehicle rats (FIG. 25, right panel). 25HC3S, at 30 mg/kg, significantly improved body weight gain after surgery while modest increases were observed at 60 mg/kg (FIG. 25, left panel). Both body temperature and body weight change measures are considered clinical indictors of improvement. No significant differences were observed in serum biochemistry analytes, including serum bilirubin (data not shown).

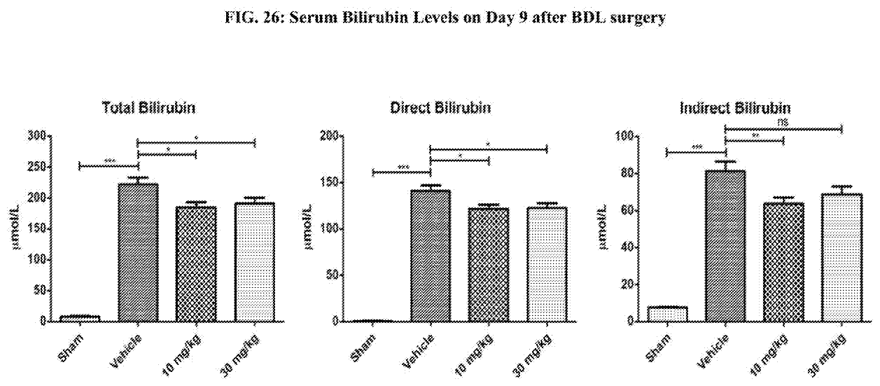

In the follow-up study, a lower dose of 25HC3S was examined for efficacy in the same BDL model by the same CRO. Rats were dosed daily with 10 or 30 mg/kg 25HC3S or Vehicle (N=8/group). The BDL surgeries were successful in subsequent studies as serum bilirubin increased approximately ˜21 to 25 fold (p<0.001) and ALT, ALP, AST and bile acids were significantly elevated in all BDL groups compared to the sham group (FIG. 26). Serum total, direct and indirect bilirubin levels were nearly all found to be significantly reduced in 25HC3 S-treated groups compared to Vehicle-treated rats (FIG. 26), while there were trends of decrease in serum liver enzymes (data not shown). Dose-dependency was not observed. Histological analyses were also performed on liver tissues but no differences were observed between the treatment groups (data not shown). For this study, body weight and temperature were not measured.

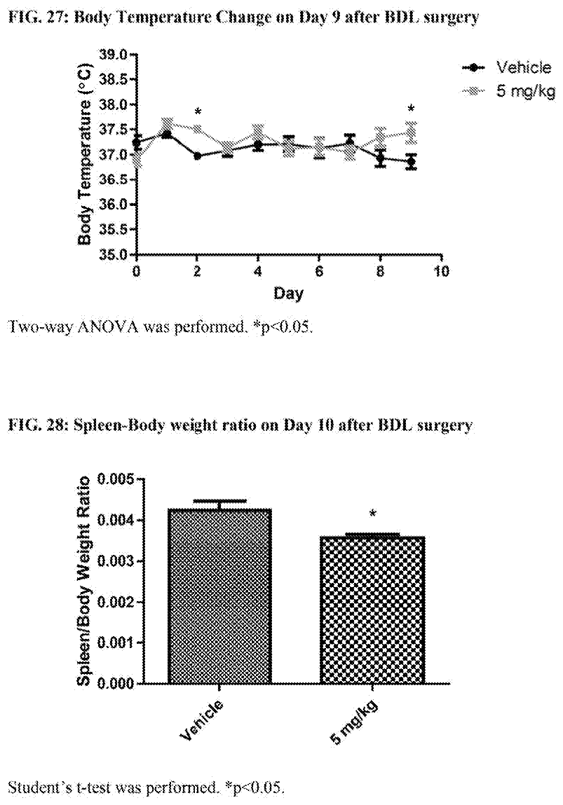

Efficacy with daily oral dosing of 5 mg/kg 25HC3S (N=10/group) was also examined. Body temperature and spleen-to-body weight ratio on Day 9 were significantly improved compared to Vehicle (FIGS. 27 and 28), while changes in body weight and other serum chemistry measures exhibited little or no differences (data not shown). The results from this study suggest that in this rodent model of cholestasis/cholangitis, the 5 mg/kg dose may not be sufficient in producing a therapeutic benefit.

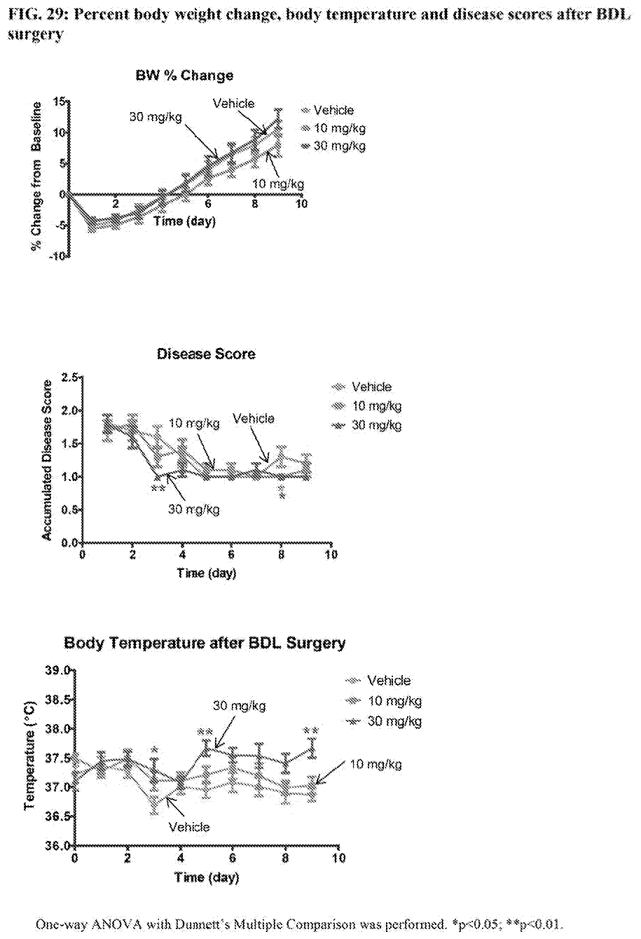

25HC3S dosing regimen was also examined for efficacy in this BDL model. Rats were dosed orally every three days with 10 or 30 mg/kg 25HC3S or Vehicle (N=10/group) starting on Day 1. Rats received a total of 3 doses of 25HC3S or Vehicle over the 9 day period (Days 1, 4 and 7). While changes in body temperature and disease scores on several days throughout the study were significant (FIG. 29), no significant differences in body weight, organ-to-bodyweight ratios or serum clinical chemistry were observed.

In summary, 25HC3S is efficacious across several dose ranges (10, 30, 60 mg/kg) to significantly ameliorate both “clinical signs” (body weight loss, body temperature loss) and serum bilirubin levels in a rodent model of cholangitis and cholestasis. Daily dosing of 25HC3S was found to be more efficacious, however, compared to dosing every 3 days in improving body weight gain, maintaining body temperature and reducing serum bilirubin.

### Example 14. Capsule Formulation Follow-Up Study

### Objective

1) To better understand the effect of drug loading and each excipient on in vitro drug release profiles.

2) To develop some formulations having faster and more reproducible dissolution than the capsule formulations described in above Example 11.

### Formulation Preparation

Eight bulk formulations were prepared in 250 mL I-Chem jars at 100 grams per batch. Each formulation jar was immersed in a water bath maintained at 50-55° C. throughout the process. Gelucire 44/14 was heated in a 60° C. oven until melted. The Gelucire was manually mixed with a spatula prior to dispensing. The melted Gelucire 44/14 was weighted out and added into each jar. Then individual excipients were weighed out and added into the melted Gelucire 44/14 with 5 minutes of overhead mixing at 400-500 rpm after each addition. Powdered 25HC3 S was slowly added into the mixture and mixed with a spatula until visually fully mixed. The bulk formulation was homogenized with setting of “2” for 3 minutes and overhead mixing at 600-700 rpm for 5 minutes. The final formulation was manually filled into size 0 HPMC capsules with 500 mg of targeted capsule fill weight to achieve 50 mg dose strength per capsule.

TABLE 28
Formulation Composition (%, w/w)

| | Drug | | | | Plurol | | Drug | Cap- |
|---|---|---|---|---|---|---|---|---|
| Formu- | Load- | Gelucire | Gelucire | | CC | Precirol | Dose | sule |
| lation | ing | 44/14 | 50/13 | Labrasol | 497 | ATO5 | (mg) | Size |
| 14-1 | 10 | 70 | 5 | 10 | 5 | 0 | 50 | 0 |
| 14-2 | 10 | 65 | 15 | 0 | 10 | 0 | 50 | 0 |
| 14-3 | 10 | 60 | 25 | 5 | 0 | 0 | 50 | 0 |
| 14-4 | 10 | 85 | 5 | 0 | 0 | 0 | 50 | 0 |
| 14-C | 10 | 88 | 0 | 0 | 0 | 2 | 50 | 0 |
| 14-5 | 10 | 75 | 5 | 10 | 0 | 0 | 50 | 0 |
| 14-6 | 10 | 75 | 15 | 0 | 0 | 0 | 50 | 0 |
| 14-7 | 10 | 75 | 7.5 | 7.5 | 0 | 0 | 50 | 0 |
| 14-8 | 10 | 80 | 5 | 5 | 0 | 0 | 50 | 0 |

### Dissolution Testing

The release rate of 25HC3S was determined using a USP Apparatus 2 dissolution tester (n=6 replicates). Dissolution medium containing 1000 mL of 0.5% Triton X-100 in 0.1N HCl was maintained at 37° C. with 75 rpm paddle speed over the course of the 4-hour dissolution test. The standard sampling time points were 0.25, 0.5, 0.75, 1, 2, and 4 hours. A 1.5 hours time point was added for the sample stored for 11 weeks at 25° C. and 60% relative humidity. A 1 mL sample was taken at each time point and assayed using HPLC.

### Results

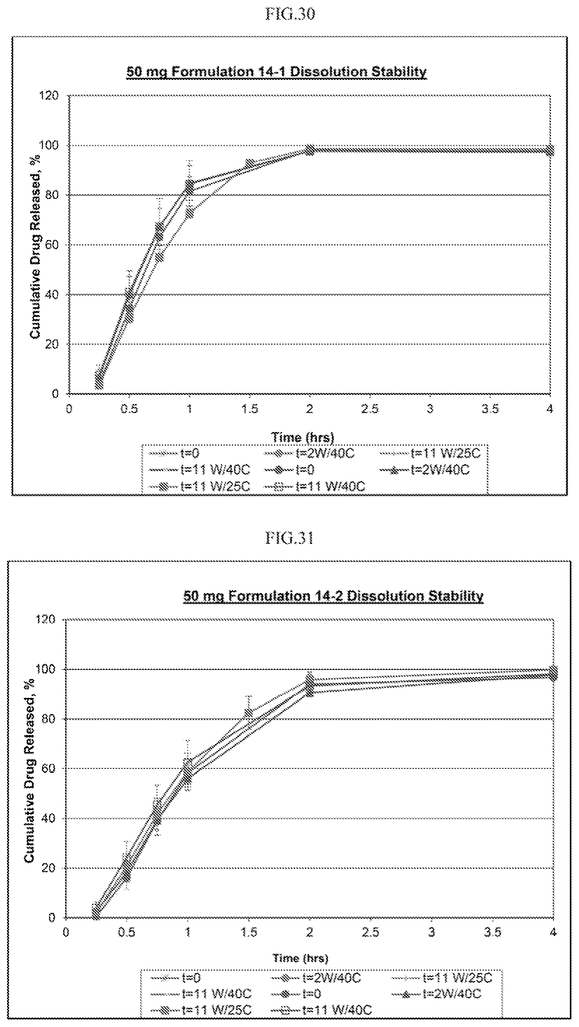

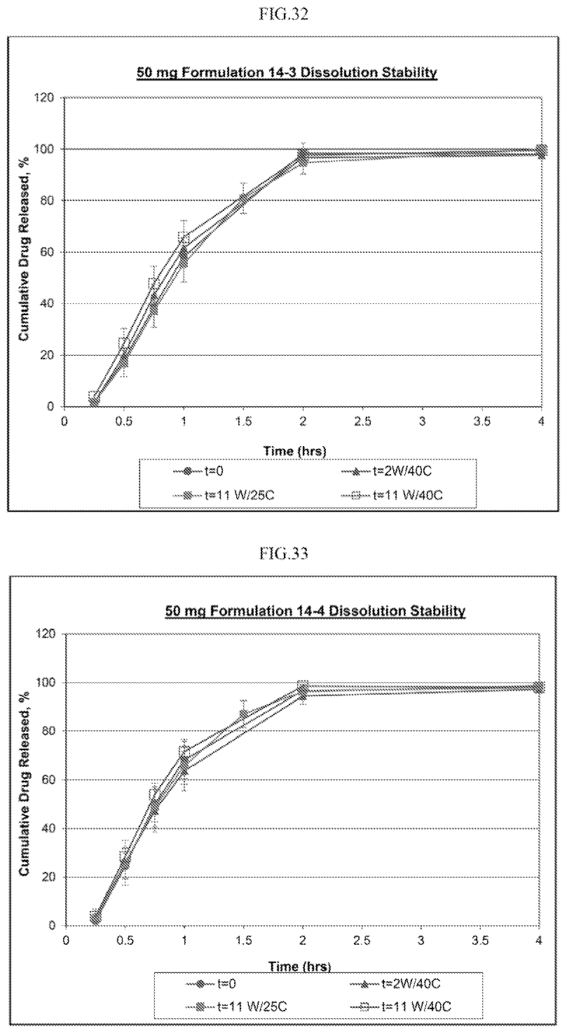

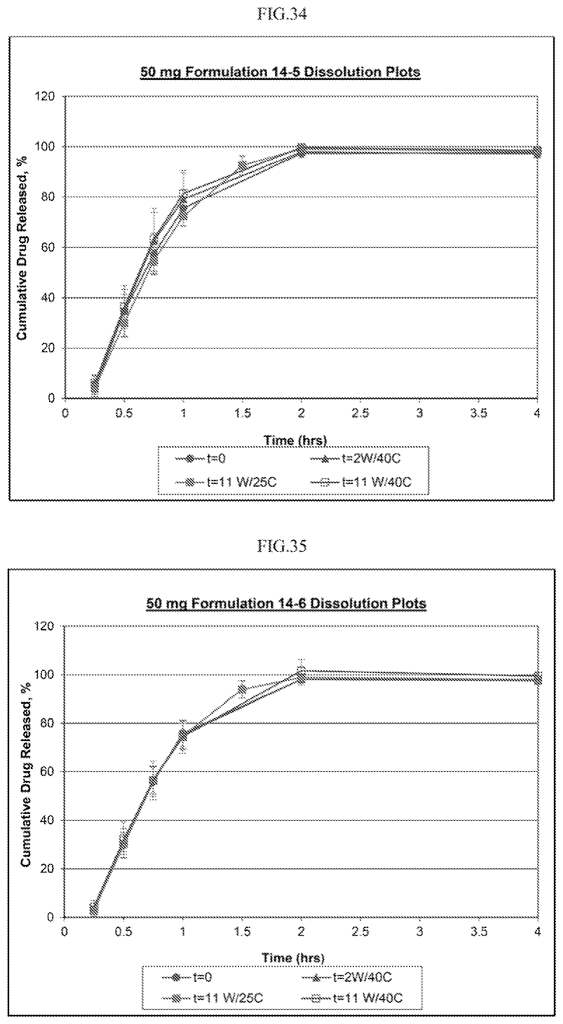

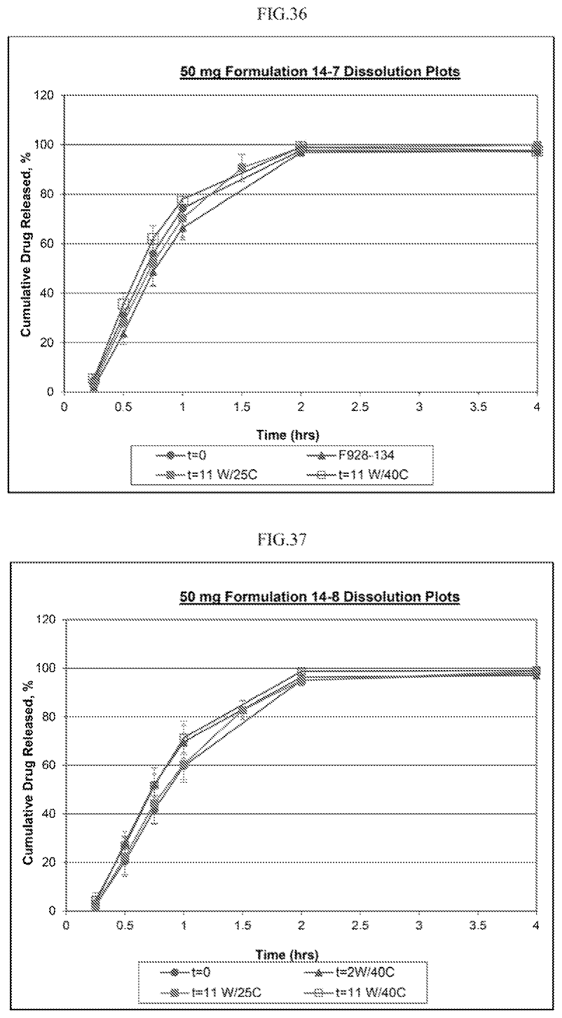

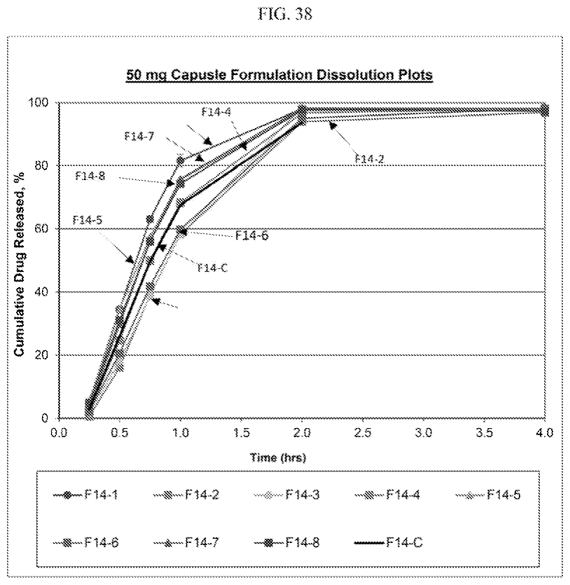

The results from the dissolution experiments for capsule formulations 14-1 to -8 are provided in FIGS. 30-37, respectively. As shown in FIGS. 30-37, the capsule formulations 14-1 to -8 were tested at t=0 and after storage at different temperatures and relative humidities for different times. FIG. 38 shows the dissolution results for capsule formulations 14-C and 14-1 to -8 at t=0.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

For all numeric ranges provided herein, it should be understood that the ranges include all integers between the highest and lowest value of the range, as well as all decimal fractions lying between those values, e.g. in increments of 0.1.

For all numeric values provided herein, the value is intended to encompass all statistically significant values surrounding the numeric value.

While the disclosure has been described in terms of its preferred embodiments, those skilled in the art will recognize that the disclosure can be practiced with modification within the spirit and scope of the appended aspects and claims. Accordingly, the present disclosure should not be limited to the embodiments as described above, but should further include all modifications and equivalents thereof within the spirit and scope of the description provided herein.

1. A composition comprising:
particles in a vehicle;
the particles comprising one or more oxygenated cholesterol sulfates (OCS), the particles having a median particle size, as measured by laser diffraction, ranging from 0.1 μm to 500 μm; and
the vehicle comprising at least one polyoxylglyceride,
wherein the composition is contained within a capsule.

2. The composition of claim 1, wherein the at least one polyoxylglyceride comprises a saturated polyglycolized glyceride.

3. The composition of claim 2, wherein the saturated polyglycolized glyceride is a saturated polyglycolized glyceride having a melting point of from about 38° C. to about 55° C. and a hydrophilic-lipophilic balance (HLB) of from about 1 to about 16.

4. The composition of claim 2, wherein the saturated polyglycolized glyceride is a saturated polyglycolized glyceride having a melting point of from about 38° C. to about 50° C. and an HLB of from about 1 to about 16.

5. The composition of claim 2, wherein the saturated polyglycolized glyceride is lauroyl polyoxylglycerides and/or stearoyl polyoxylglycerides.

6. The composition of claim 1, wherein the at least one polyoxylglyceride is present in the composition in an amount ranging from about 10 wt % to about 99 wt %, based on total weight of the composition.

7. (canceled)

8. The composition of claim 1, wherein the composition comprises a suspension of the particles in the vehicle.

9. (canceled)

10. The composition of claim 1, wherein the one or more oxygenated cholesterol sulfates comprises 5-cholesten-3β, 25-diol, 3-sulfate (25HC3S) or a pharmaceutically acceptable salt thereof.

11. The composition of claim 1, wherein the one or more oxygenated cholesterol sulfates is present in an amount ranging from about 0.5 wt % to about 50 wt %, based on weight of the composition.

12. The composition of claim 1, further comprising at least one surfactant.

13.-15. (canceled)

16. The composition of claim 12, wherein the at least one surfactant is present in the composition in an amount ranging from about 0.01 wt % to about 20 wt %, based on weight of the composition.

17. The composition of claim 12, wherein the at least one surfactant is present in the composition in an amount ranging from about 0.01 wt % to about 10 wt %, based on weight of the composition.

18. The composition of claim 1, further comprising at least one polyglyceryl fatty acid ester present in the composition in an amount ranging from about 1 wt % to about 15 wt %, based on total weight of the composition.

19. (canceled)

20. The composition of claim 1, wherein the composition is contained within a capsule.

21. The composition of claim 1, comprising:
particles comprising 25HC3S or pharmaceutically acceptable salt thereof;
lauroyl polyoxylglycerides; and
stearoyl polyoxylglycerides.

22.-28. (canceled)

29. A method of treating, in a subject in need thereof, at least one of: hyperlipidemia or a disease or condition caused by hyperlipidemia; dysfunction or failure of at least one organ; a lipid metabolism disorder; metabolic disorder; atherosclerosis; injury caused by ischemia; unwanted cell death; sepsis; acute radiation syndrome; a liver disorder; a lipid accumulation disorder; a skin lesion; and an inflammatory skin disease; the method comprising
administering to the subject a therapeutically effective amount of a composition comprising:
particles in a vehicle;
the particles comprising one or more oxygenated cholesterol sulfates (OCS), the particles having a median particle size, as measured by laser diffraction, ranging from 0.1 μm to 500 μm; and
the vehicle comprising at least one polyoxylglyceride,
wherein the composition is contained within a capsule.

30. The method of claim 29, wherein the administering is performed orally.

31.-33. (canceled)

34. The composition of claim 1, wherein the particles have a median particle size, as measured by laser diffraction, ranging from 0.25 μm to 50 μm.

35. The composition of claim 1, wherein the particles have a median particle size, as measured by laser diffraction, ranging from 0.5 μm to 25 μm.

36. The method of claim 29, wherein the particles have a median particle size, as measured by laser diffraction, ranging from 0.25 μm to 50 μm.
